(19) United States
(12) Patent Application Publication
(10) Pub. No.: US 2025/0154246 A1
(43) Pub. Date: May 15, 2025

(54) NOVEL T CELL RECEPTORS AND IMMUNE THERAPY USING THE SAME

(71) Applicant: Immatics Biotechnologies GmbH, Tübingen (DE)

(72) Inventors: Claudia WAGNER, Tübingen (DE); Leonie ALTEN, Tübingen (DE); Sebastian BUNK, Tübingen (DE); Dominik MAURER, Tübingen (DE)

(21) Appl. No.: 18/916,878

(22) Filed: Oct. 16, 2024

Related U.S. Application Data
(63) Continuation of application No. 17/397,656, filed on Aug. 9, 2021, now abandoned.
Application No. 17/397,656 is a continuation of application No. 16/733,845, filed on Jan. 3, 2020, now Pat. No. 11,111,294.
Application No. 16/733,845 is a continuation of application No. 16/023,731, filed on Jun. 29, 2018, now Pat. No. 10,590,194.
(60) Provisional application No. 62/527,844, filed on Jun. 30, 2017.

(30) Foreign Application Priority Data
Jun. 30, 2017 (DE) 102017114737.3

(51) Int. Cl. C07K 16/26; A61K 40/11; A61K 40/32; A61K 40/42; C07K 14/725; C07K 14/81; C07K 16/28; C07K 16/38; C12N 5/0783; C12N 15/85

(52) U.S. Cl. CPC C07K 16/26; A61K 40/11; A61K 40/32; A61K 40/42; C07K 14/7051; C07K 14/8135; C07K 16/2833; C07K 16/38; C12N 5/0636; C12N 15/85; C07K 2317/30; C07K 2317/32; C07K 2317/565; C07K 2317/62; C12N 2015/8518

(57) ABSTRACT

The present invention pertains to antigen recognizing constructs against tumor associated antigens (TAA), in particular the TAA Serine protease inhibitor Kazal-type 2 (SPINK2). The invention in particular provides novel T cell receptor (TCR) based molecules which are selective and specific for the tumor expressed antigen of the invention. The TCR of the invention, and SPINK2 binding fragments derived therefrom, are of use for the diagnosis, treatment and prevention of SPINK2 expressing cancerous diseases. Further provided are nucleic acids encoding the antigen recognizing constructs of the invention, vectors comprising these nucleic acids, recombinant cells expressing the antigen recognizing constructs and pharmaceutical compositions comprising the compounds of the invention.

### CROSS-REFERENCE TO RELATED APPLICATIONS

This application is a continuation of U.S. patent application Ser. No. 17/397,656, filed on 9 Aug. 2021, which is a continuation of U.S. patent application Ser. No. 16/733,845, filed 3 Jan. 2020, now U.S. Pat. No. 11,111,294, issued 7 Sep. 2021, which is a continuation of U.S. patent application Ser. No. 16/023,731, filed 29 Jun. 2018, now U.S. Patent No. 10,590, 194, issued 17 Mar. 2020, which claims priority to 62/527,844, filed 30 Jun. 2017 and German Patent Application No. 10 2017 114 737.3, filed 30 Jun. 2017. The disclosures of the priority applications are incorporated in their entirety herein by reference.

This application is also related to PCT/EP2018/067380, filed 28 Jun. 2018, the content of which is incorporated herein by reference in its entirety.

### REFERENCE TO SEQUENCE LISTING SUBMITTED AS AN XML FILE (.xml)

A Sequence Listing is submitted herewith as an XML file named “3000058-010005_Sequence_Listing_ST26” created on 15 Oct. 2024 and having a size of 143,939 bytes as permitted under 37 C.F.R. § 1.821 (c). The material in the aforementioned file is hereby incorporated by reference in its entirety.

### FIELD OF THE INVENTION

The present invention pertains to antigen recognizing constructs against tumor associated antigens (TAA), in particular the TAA Serine protease inhibitor Kazal-type 2 (SPINK2). The invention in particular provides novel T cell receptor (TCR) based molecules which are selective and specific for the tumor expressed antigen of the invention. The TCR of the invention, and SPINK2 binding fragments derived therefrom, are of use for the diagnosis, treatment and prevention of SPINK2 expressing cancerous diseases. Further provided are nucleic acids encoding the antigen recognizing constructs of the invention, vectors comprising these nucleic acids, recombinant cells expressing the antigen recognizing constructs and pharmaceutical compositions comprising the compounds of the invention.

### DESCRIPTION OF RELATED ART

Serine protease inhibitor Kazal-type 2 (SPINK2), is also known as acrosin-trypsin inhibitor, and is a protein that in humans is encoded by the SPINK2 gene. The encoded protein acts as a trypsin and acrosin inhibitor in the genital tract and is localized in the spermatozoa. The protein has been associated with the progression of lymphomas. Alternative splicing results in multiple transcript variants.

T-cell based immunotherapy targets represent peptide epitopes derived from tumor-associated or tumor-specific proteins, which are presented by molecules of the major histocompatibility complex (MHC). These tumor associated antigens (TAAs) can be peptides derived from all protein classes, such as enzymes, receptors, transcription factors, etc. which are expressed and, as compared to unaltered cells of the same origin, usually up-regulated in cells of the respective tumor.

Specific elements of the cellular immune response are capable of specifically recognizing and destroying tumor cells. The isolation of T-cells from tumor-infiltrating cell populations or from peripheral blood suggests that such cells play an important role in natural immune defense against cancer. CD8-positive T-cells in particular, which recognize class I molecules of the major histocompatibility complex (MHC)-bearing peptides of usually 8 to 10 amino acid residues derived from proteins or defective ribosomal products (DRiPs) located in the cytosol, play an important role in this response. The MHC-molecules of the human are also designated as human leukocyte-antigens (HLA).

There are two classes of MHC-molecules, MHC class I and MHC class II. Complexes of peptide and MHC class I are recognized by CD8-positive T-cells bearing the appropriate T-cell receptor (TCR), whereas complexes of peptide and MHC class Il molecules are recognized by CD4-positive-helper-T-cells bearing the appropriate TCR. Since both types of response, CD8 and CD4 dependent, contribute jointly and synergistically to the anti-tumor effect, the identification and characterization of tumor-associated antigens and corresponding T cell receptors is important in the development of cancer immunotherapies such as vaccines and cell therapies.

In the MHC class I dependent immune reaction, peptides not only have to be able to bind to certain MHC class I molecules expressed by tumor cells, they subsequently also have to be recognized by T-cells bearing specific T-cell receptors (TCR). Therefore, TAAs are a starting point for the development of a T-cell based therapy including but not limited to tumor vaccines and cell therapies.

Approximately 90 percent of peripheral blood T cells express a TCR consisting of an α polypeptide and a β polypeptide. A small percentage of T cells (about 5% of total T cells) have been shown to express a TCR consisting of a γ polypeptide and a δ polypeptide. γδ T cells are found at their highest abundance in the gut mucosa, within a population of lymphocytes known as intraepithelial lymphocytes (IELs). The antigenic molecules that activate yo γδ cells are still widely unknown. However, γδ T cells are not MHC restricted and seem to be able to recognize whole proteins rather than requiring peptides to be presented by MHC molecules on antigen presenting cells, although some recognize MHC class IB molecules. Human Vγ9/Vδ2 T cells, which constitute the major γδ T cell population in peripheral blood, are unique in that they specifically and rapidly respond to a small non-peptidic microbial metabolite, HMB-PP, an isopentenyl pyro-phosphate precursor. Estimates of the percentages of T cells that may be found in peripheral blood from healthy donors are as follows: CD3+=70.78%±4.71; CD3+CD4=38.97%±5.66; CD3+CD8=28.955%±7.43; CD3+CD56+=5.22%±1.74; CD3−CD56+=10.305%±4.7; CD3+CD45RA=45.00%±7.19; CD3+CD45RO+=27.21%±7.34.

The chains of the T cell antigen receptor of a T cell clone are each composed of a unique combination of domains designated variable (V), [diversity (D),] joining (J), and constant (C). In each T cell clone, the combination of V, D and J domains of both the alpha and the beta chains or of both the delta and gamma chains participates in antigen recognition in a manner which is uniquely characteristic of that T cell clone and defines a unique binding site, also known as the idiotype of the T cell clone. In contrast, the C domain does not participate in antigen binding.

A TCR is a heterodimeric cell surface protein of the immunoglobulin super-family, which is associated with invariant proteins of the CD3 complex involved in mediating signal transduction. TCRs exist in αβ and γδ forms, which are structurally similar but have quite distinct anatomical locations and probably functions. The extracellular portion of native heterodimeric αβTCR and γδTCR each contain two polypeptides, each of which has a membrane-proximal constant domain, and a membrane-distal variable domain. Each of the constant and variable domains includes an intra-chain disulfide bond. The variable domains contain the highly polymorphic loops analogous to the complementarity determining regions (CDRs) of antibodies. The use of TCR gene therapy overcomes a number of current hurdles. It allows equipping patients' own T cells with desired specificities and generation of sufficient numbers of T cells in a short period of time, avoiding their exhaustion. The TCR will be transduced into central memory T cells or T cells with stem cell characteristics, which may ensure better persistence and function upon transfer. TCR-engineered T cells will be infused into cancer patients rendered lymphopenic by chemotherapy or irradiation, allowing efficient engraftment but inhibiting immune suppression.

While advances have been made in the development of molecular-targeting drugs for cancer therapy, there remains a need in the art to develop new anti-cancer agents that specifically target molecules highly specific to cancer cells. The present description addresses that need by providing novel SPINK2-001 TCRs, respective recombinant TCR constructs, nucleic acids, vectors and host cells that specifically bind TAA epitope(s) as disclosed; and methods of using such molecules in the treatment of cancer. The term TAA in context of the invention relates in particular to the protein SPINK2 and even more preferably to the epitope SPINK2-001 as also disclosed herein elsewhere.

### SUMMARY

### Antigen Recognizing Constructs

The object of the invention is solved in a first aspect by an antigen recognizing construct comprising at least one complementary determining region (CDR) 3 having at least 50%, 60%, 70%, 80%, 90%, 95%, 98%, 99%, or preferably 100% sequence identity to an amino acid sequence selected from SEQ ID NOs: 3, 9, 15, 21, 27, 33, 39, 45, 51, 57, 63, 69, 75, 81, 87, 93, 99, 105, 111, and 117.

### BRIEF DESCRIPTION OF THE DRAWINGS

FIG. 1 depicts IFNγ release from CD8+ T-cells electroporated with alpha and beta chain RNA of TCR R39P1C12 (Table 1) after co-incubation with T2 target cells loaded with SPINK2-001 peptide (SEQ ID NO:133) or various SPINK2-001 alanine- or threonine-substitution variants at positions 1-9 of SEQ ID NO: 133 (SEQ ID NOs: 134-149) or control peptide NYESO1-001 (SEQ ID NO:159).

FIG. 2 depicts IFNγ release from CD8+ T-cells electroporated with alpha and beta chain RNA of TCR R39P1F5 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOs:134-149) or control peptide NYESO1-001 (SEQ ID NO:159).

FIG. 3 depicts IFNγ release from CD8+ T-cells electroporated with alpha and beta chain RNA of TCR R40P1C2 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOS:134-149) or control peptide NYESO1-001 (SEQ ID NO:159).

FIG. 4 depicts IFNγ release from CD8+ T-cells electroporated with alpha and beta chain RNA of TCR R41P3E6 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOs:134-149) or control peptide NYESO1-001 (SEQ ID NO:159).

FIG. 5 depicts IFNγ release from CD8+ T-cells electroporated with alpha and beta chain RNA of TCR R43P3G4 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOs:134-149) or control peptide NYESO1-001 (SEQ ID NO:159).

FIG. 6 depicts IFNγ release from CD8+ T-cells electroporated with alpha and beta chain RNA of TCR R44P3B3 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOs:134-149) or control peptide NYESO1-001 (SEQ ID NO:159).

FIG. 7 depicts IFNγ release from CD8+ T-cells electroporated with alpha and beta chain RNA of TCR R44P3E7 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOs:134-149) or control peptide NYESO1-001 (SEQ ID NO:159).

FIG. 8 depicts IFNγ release from CD8+ T-cells electroporated with alpha and beta chain RNA of TCR R49P2B7 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOS:134-149) or control peptide NYESO1-001 (SEQ ID NO:159).

FIG. 9 depicts IFNγ release from CD8+ T-cells electroporated with alpha and beta chain RNA of TCR R55P1G7 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOs:134-149) or control peptide NYESO1-001 (SEQ ID NO:159).

FIG. 10 depicts IFNγ release from CD8+ T-cells electroporated with alpha and beta chain RNA of TCR R59P2A7 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOS:134-149) or control peptide NYESO1-001 (SEQ ID NO:159).

FIG. 11 depicts IFNγ release from CD8+ T-cells electroporated with alpha and beta chain RNA of TCRs R39P1C12 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 12 depicts IFNγ release from CD8+ T-cells electroporated with alpha and beta chain RNA of TCRs R39P1F5 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 13 depicts IFNγ release from CD8+ T-cells electroporated with alpha and beta chain RNA of TCRs R40P1C2 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 14 depicts IFNγ release from CD8+ T-cells electroporated with alpha and beta chain RNA of TCRs R41P3E6 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 15 depicts IFNγ release from CD8+ T-cells electroporated with alpha and beta chain RNA of TCRs R43P3G4 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 16 depicts IFNγ release from CD8+ T-cells electroporated with alpha and beta chain RNA of TCRs R44P3B3 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 17 depicts IFNγ release from CD8+ T-cells electroporated with alpha and beta chain RNA of TCRs R44P3E7 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 18 depicts IFNγ release from CD8+ T-cells electroporated with alpha and beta chain RNA of TCRs R49P2B7 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 19 depicts IFNγ release from CD8+ T-cells electroporated with alpha and beta chain RNA of TCRs R55P1G7 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 20 depicts IFNγ release from CD8+ T-cells electroporated with alpha and beta chain RNA of TCRs R59P2A7 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO:155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159).

FIG. 21 depicts IFNγ release from CD8+ T-cells electroporated with alpha and beta chain RNA of TCR R39P1C12 (Table 1) after co-incubation with T2 target cells loaded with SPINK2-001 peptide (SEQ ID NO:133) in various peptide loading concentrations from 10 μM to 10 pM.

FIG. 22 depicts IFNγ release from CD8+ T-cells electroporated with alpha and beta chain RNA of TCR R39P1F5 (Table 1) after co-incubation with T2 target cells loaded with SPINK2-001 peptide (SEQ ID NO:133) in various peptide loading concentrations from 10 μM to 10 pM.

FIG. 23 depicts IFNγ release from CD8+ T-cells electroporated with alpha and beta chain RNA of TCR R40P1C2 (Table 1) after co-incubation with T2 target cells loaded with SPINK2-001 peptide (SEQ ID NO:133) in various peptide loading concentrations from 10 μM to 10 pM.

FIG. 24 depicts IFNγ release from CD8+ T-cells electroporated with alpha and beta chain RNA of TCR R41P3E6 (Table 1) after co-incubation with T2 target cells loaded with SPINK2-001 peptide (SEQ ID NO:133) in various peptide loading concentrations from 10 μM to 10 pM.

FIG. 25 depicts IFNγ release from CD8+ T-cells electroporated with alpha and beta chain RNA of TCR R43P3G4 (Table 1) after co-incubation with T2 target cells loaded with SPINK2-001 peptide (SEQ ID NO:133) in various peptide loading concentrations from 10 μM to 10 pM.

FIG. 26 depicts IFNγ release from CD8+ T-cells electroporated with alpha and beta chain RNA of TCR R44P3B3 (Table 1) after co-incubation with T2 target cells loaded with SPINK2-001 peptide (SEQ ID NO:133) in various peptide loading concentrations from 10 μM to 10 pM.

FIG. 27 depicts IFNγ release from CD8+ T-cells electroporated with alpha and beta chain RNA of TCR R44P3E7 (Table 1) after co-incubation with T2 target cells loaded with SPINK2-001 peptide (SEQ ID NO:133) in various peptide loading concentrations from 10 μM to 10 pM.

FIG. 28 depicts IFNγ release from CD8+ T-cells electroporated with alpha and beta chain RNA of TCR R49P2B7 (Table 1) after co-incubation with T2 target cells loaded with SPINK2-001 peptide (SEQ ID NO:133) in various peptide loading concentrations from 10 μM to 10 pM.

FIG. 29 depicts IFNγ release from CD8+ T-cells electroporated with alpha and beta chain RNA of TCR R55P1G7 (Table 1) after co-incubation with T2 target cells loaded with SPINK2-001 peptide (SEQ ID NO:133) in various peptide loading concentrations from 10 μM to 10 pM.

FIG. 30 depicts IFNγ release from CD8+ T-cells electroporated with alpha and beta chain RNA of TCR R59P2A7 (Table 1) after co-incubation with T2 target cells loaded with SPINK2-001 peptide (SEQ ID NO:133) in various peptide loading concentrations from 10 μM to 10 pM.

FIG. 31 depicts HLA-A*02/SPINK2-001 tetramer or HLA-A*02/NYESO1-001 tetramer staining, respectively, of CD8+ T-cells electroporated with alpha and beta chain RNA of TCRs R41P3E6, R44P3B3, R49P2B7 and R55P1G7, respectively.

FIG. 32 depicts IFNγ release from CD8+ T-cells electroporated with alpha and beta chain RNA of TCR R55P1G7 (A) or R44P3B3 (B) (Table 1) after co-incubation with A375 target cells overexpressing SPINK2 or wildtype A375 cells.

FIGS. 33A and 33B depict biolayer interferometry binding analysis of HLA-A*02:01:SPINK2-001 (immobilized on sensor) and soluble TCR variants of TCRs R39P1F5, R39P1C12, R41P3E6, R44P3B3 and R55P1G7, specifically, the signal (y-axis) of binding curves and fits for different TCR concentrations over time (x-axis); and the response at the equilibrium (y-axis) over different TCR concentrations (x-axis) and fits, respectively.

### DETAILED DESCRIPTION OF A PREFERRED EMBODIMENT

In some embodiments the antigen recognizing construct of the invention specifically binds to a TAA-peptide-HLA molecule complex, wherein the TAA peptide comprises, or alternatively consists of, a variant of the TAA which is at least 66%, preferably at least 77%, and more preferably at least 88% homologous (preferably at least 77% or at least 88% identical) to the amino acid sequence of the TAA of the invention, wherein said variant binds to an HLA class I or class II molecule and/or induces T-cells cross-reacting with said peptide, or a pharmaceutically acceptable salt thereof, wherein said peptide is not the underlying full-length polypeptide.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site). In a particular embodiment, for example when comparing the protein or nucleic acid sequence of an antigen recognizing construct of the invention to another protein/gene, the percentage identity can be determined by the Blast searches supported at the NCBI web site; in particular for amino acid identity, those using BLASTP with the following parameters: Expected threshold 10; Word size: 6; Matrix: BLOSUM62; Gap Costs: Existence: 11, Extension: 1; Neighboring words threshold: 11; Compositional adjustments: Conditional compositional score matrix adjustment.

In the context of the present invention it shall be understood that any embodiments referred to as “comprising” certain features of the invention, shall be understood to include in some more preferred embodiments the more restricted description of “consisting of” or “consisting essentially of” the very same features of the present invention.

In another additional or alternative embodiment, the antigen recognizing construct may further comprise a CDR1 and/or a CDR2 domain sequence. Within the variable domain, CDR1 and CDR2 are found in the variable (V) region of a polypeptide chain, and CDR3 includes some of V, all of diversity (D) and joining (J) regions. CDR3 is the most variable and is the main CDR responsible for specifically and selectively recognizing an antigen. CDR1 and CDR2 sequences may be selected from a CDR sequence of a human variable chain allele.

Native alpha-beta heterodimeric TCRs have an alpha chain and a beta chain. Each chain comprises variable, joining and constant regions, and the beta chain also usually contains a short diversity region between the variable and joining regions, but this diversity region is often considered as part of the joining region. Each variable region comprises three CDRs (Complementarity Determining Regions) embedded in a framework sequence, one being the hypervariable region named CDR3. There are several types of alpha chain variable (Vα) regions and several types of beta chain variable (Vβ) regions distinguished by their framework, CDR1 and CDR2 sequences, and by a partly defined CDR3 sequence. The Vα types are referred to in IMGT nomenclature by a unique TRAV number, Vβ types are referred to by a unique TRBV number. For more information on immunoglobulin antibody and TCR genes see the international ImMunoGeneTics information system®, Lefranc M-P et al (Nucleic Acids Res. 2015 January;43 (Database issue): D413-22; and http://www.imgt.org/).

Therefore, in one additional or alternative embodiment the antigen recognizing construct of the invention comprises CDR1, CDR2 and CDR3 sequences in a combination as provided in table 1 herein below, which display the respective variable chain allele together with the CDR3 sequence. Therefore, preferred are antigen recognizing constructs of the invention which comprise at least one, preferably, all three CDR sequences CDR1, CDR2 and CDR3. Preferably, an antigen recognizing construct of the invention comprises the respective CDR1 to CDR3 of one individual herein disclosed TCR variable region of the invention (see table 1 herein below and the example section).

The term “specificity” or “antigen specificity” or “specific for” a given antigen, as used herein means that the antigen recognizing construct can specifically bind to said antigen, preferably a TAA antigen, more preferably with high avidity, when said antigen is presented by HLA, preferably by HLA-A*02. For example, a TCR, as antigen recognizing construct, may be considered to have “antigenic specificity” for the TAA, if T cells expressing the TCR and contacted with a TAA presenting HLA secrete at least about 200 pg/ml or more (e.g., 250 pg/ml or more, 300 pg/ml or more, 400 pg/ml or more, 500 pg/ml or more, 600 pg/ml or more, 700 pg/ml or more, 1000 pg ml or more, 2,000 pg/ml or more, 2,500 pg/ml or more, 5,000 pg/ml or more) of interferon γ (IFN-γ) upon co-culture with target cells pulsed with a low concentration of a TAA antigen, such as the TAA epitopes and antigens provided herein below (e.g., about 10⁻¹¹ mol/l, 10⁻¹⁰ mol/l, 10⁻⁹ mol/l, 10⁻⁸ mol/l, 10⁻⁷ mol/l, 10⁻⁶ mol/l, 10⁻⁵ mol/l). Alternatively, or additionally, a TCR may be considered to have “antigenic specificity” for the TAA, if T cells expressing the TCR secrete at least twice as much IFN-γ as the untransduced background level of IFN-γ upon co-culture with target cells pulsed with a low concentration of the TAA antigens. Such a “specificity” as described above can—for example—be analyzed with an ELISA.

In one alternative or additional embodiment of the invention, the antigen recognizing construct selectively binds to a TAA derived antigenic peptide; preferably wherein the TAA antigenic peptide is a protein epitope or peptide having an amino acid sequence shown in table 2 below, in particular the antigenic peptide is the SPINK2-001 peptide (SEQ ID NO:133), or a variant thereof, wherein the variant is an amino acid deletion, addition, insertion or substitution of not more than three, preferably two and most preferably not more than one amino acid position. Preferred variants of SPINK2-001 are shown in table 2 below.

The term “selectivity” or “selective recognizing/binding” is understood to refer to the property of an antigen recognizing construct, such as a TCR or antibody, to selectively recognize or bind to preferably only one specific epitope and preferably shows no or substantially no cross-reactivity to another epitope. Preferably “selectivity” or “selective recognizing/binding” means that the antigen recognizing construct (e.g. a TCR) selectively recognizes or binds to preferably only one specific epitope and preferably shows no or substantially no cross-reactivity to another epitope, wherein said epitope is unique for one protein, such that the antigen recognizing construct shows no or substantially no cross-reactivity to another epitope and another protein.

The antigen recognizing construct according to the invention is preferably selected from an antibody, or derivative or fragment thereof, or a T cell receptor (TCR), or derivative or fragment thereof. A derivative or fragment of an antibody or TCR of the invention shall preferably retain the antigen binding/recognizing ability of the parent molecule, in particular its specificity and/or selectivity as explained above. Such binding functionality may be retained by the presence of a CDR3 region as defined herein.

In an embodiment of the invention, the inventive TCRs are able to recognize TAA antigens in a major histocompatibility complex (MHC) class I-dependent manner. “MHC class I-dependent manner,” as used herein, means that the TCR elicits an immune response upon binding to TAA antigens within the context of an MHC class I molecule. The MHC class I molecule can be any MHC class I molecule known in the art, e.g., HLA-A molecules. In a preferred embodiment of the invention, the MHC class I molecule is an HLA-A*02 molecule.

In an aspect, the TCR-elicited immune response or T-cell response may refer to the proliferation and activation of effector functions induced by a peptide in vitro or in vivo. For MHC class I restricted cytotoxic T cells, for example, effector functions may be lysis of peptide-pulsed, peptide-precursor pulsed or naturally peptide-presenting target cells, secretion of cyto-kines, preferably Interferon-gamma, TNF-alpha, or IL-2 induced by peptide, secretion of effector molecules, for example, granzymes or perforins induced by peptide, or degranulation.

In an aspect, the TCR-elicited immune response or T-cell response may refer to the proliferation and activation of effector functions induced by a peptide in vitro or in vivo. For MHC class I restricted cytotoxic T cells, for example, effector functions may be lysis of peptide-pulsed, peptide-precursor pulsed or naturally peptide-presenting target cells, secretion of cytokines, preferably Interferon-gamma, TNF-alpha, or IL-2 induced by peptide, secretion of effector molecules, for example, granzymes or perforins induced by peptide, or degranulation.

The invention provides both single chain antigen recognizing construct and double chain recognizing constructs.

In an embodiment, the TCR alpha variable domain has at least one mutation relative to a TCR alpha domain shown in Table 1; and/or the TCR beta variable domain has at least one mutation relative to a TCR alpha domain shown in Table 1. In an embodiment, a TCR comprising at least one mutation in the TCR alpha variable domain and/or TCR beta variable domain has a binding affinity for, and/or a binding half-life for, a TAA peptide-HLA molecule complex, which is at least double that of a TCR comprising the unmutated TCR alpha domain and/or unmutated TCR beta variable domain.

The TCR alpha chains of the present description may further comprise a TCR alpha transmembrane domain and/or a TCR alpha intracellular domain. The TCR beta chains of the present description may further comprise a TCR beta transmembrane domain and/or a TCR beta intracellular domain.

The invention in particular provides a TCR as antigen recognizing construct, or fragment or derivative thereof. The TCR preferably is of human, which is understood as being generated from a human TCR locus and therefore comprising human TCR sequences. Furthermore, the TCR of the invention may be characterized in that it is of human origin and specifically recognizes a TAA antigen of the invention.

Another embodiment of the invention additionally or alternatively provides the antigen recognizing construct described above, which induces an immune response, preferably wherein the immune response is characterized by an increase in interferon (IFN) γ levels.

TCRs of the invention may be provided as single chain α or β, or γ and δ, molecules, or alternatively as double chain constructs composed of both the α and β chain, or γ and δ chain. Furthermore, if in context of the present invention any one, two or all CDR regions of a given TCR as described in the present invention, is grafted from one TCR chain type into another, it is in some embodiments preferred that such CDR originally derived from an α chain could be grafted into a γ or δ chain, and/or such CDR of a β chain could be grafted into a γ or δ chain framework. Thereby it is possible if starting with CDR sequences of an α/β TCR, one can obtain a γ/δ TCR, however, in two ways. One is by grafting the CDR from α into a γ framework and from β into a δ framework. A second, as described above, by grafting the CDR from an α into a δ framework, and from β into a γ framework. The person of skill is aware of the similarity of the framework regions between the α/β, and γ/δ TCR chains and thus, such embodiments are encompassed by the present invention (see also Lefranc M-P et al, Nucleic Acids Res. 2015 January;43 (Database issue):D413-22; and http://www.imgt.org/).

The antigen recognizing construct of the invention may comprise a TCR α or γ chain; and/or a TCR β or δ chain; wherein the TCR α or γ chain comprises a CDR3 having at least 50%, 60%, 70%, 80%, 90%, 95%, 98%, 99%, or 100% sequence identity to an amino acid sequence selected from SEQ ID NOs: 3, 15, 27, 39, 51, 63, 75, 87, 99, and 111, and/or wherein the TCR β or δ chain comprises a CDR3 having at least 50%, 60%, 70%, 80%, 90%, 95%, 98%, 99%, or 100% sequence identity to an amino acid sequence selected from SEQ ID NOs: 9, 21, 33, 45, 57, 69, 81, 93, 105, and 117.

Most preferably, in some additional embodiments, wherein the disclosure refers to antigen recognizing constructs comprising any one, two or all of the CDR1 to CDR3 regions of the herein disclosed TCR chains (see Table 1), such antigen recognizing constructs may be preferred, which comprise the respective CDR sequence of the invention with not more than three, two, and preferably only one, modified amino acid residues. A modified amino acid residue may be selected from an amino acid insertion, deletion or substitution. Most preferred is that the three, two, preferably only one modified amino acid residue is the first or last amino acid residue of the respective CDR sequence. If the modification is a substitution then it is preferable in some embodiments that the substitution is a conservative amino acid substitution.

If the antigen recognizing construct of the invention is composed of at least two amino acid chains, such as a double chain TCR, or antigen binding fragment thereof, the antigen recognizing construct may comprises in a first polypeptide chain the amino acid sequence according to SEQ ID NO: 3, and in a second polypeptide chain the amino acid sequence according to SEQ ID NO: 9; or in a first polypeptide chain the amino acid sequence according to SEQ ID NO: 15, and in a second polypeptide chain the amino acid sequence according to SEQ ID NO: 21; or in a first polypeptide chain the amino acid sequence according to SEQ ID NO: 27, and in a second polypeptide chain the amino acid sequence according to SEQ ID NO: 33; or in a first polypeptide chain the amino acid sequence according to SEQ ID NO: 39, and in a second polypeptide chain the amino acid sequence according to SEQ ID NO: 45; or in a first polypeptide chain the amino acid sequence according to SEQ ID NO: 51, and in a second polypeptide chain the amino acid sequence according to SEQ ID NO: 57; or in a first polypeptide chain the amino acid sequence according to SEQ ID NO: 63, and in a second polypeptide chain the amino acid sequence according to SEQ ID NO: 69; or in a first polypeptide chain the amino acid sequence according to SEQ ID NO: 75, and in a second polypeptide chain the amino acid sequence according to SEQ ID NO: 81; or in a first polypeptide chain the amino acid sequence according to SEQ ID NO: 87, and in a second polypeptide chain the amino acid sequence according to SEQ ID NO: 93; or in a first polypeptide chain the amino acid sequence according to SEQ ID NO: 99, and in a second polypeptide chain the amino acid sequence according to SEQ ID NO: 105; or in a first polypeptide chain the amino acid sequence according to SEQ ID NO: 111, and in a second polypeptide chain the amino acid sequence according to SEQ ID NO: 117. Any one of the aforementioned double chain TCR, or antigen binding fragments thereof, are preferred TCR of the present invention. In some embodiments, the CDR3 of the double chain TCR of the invention may be mutated. Mutations of the CDR3 sequences as provided above preferably include a substitution, deletion, addition, or insertion of not more than three, preferably two, and most preferably not more than one amino acid residue. In some embodiments, the first polypeptide chain may be a TCR α or γ chain, and the second polypeptide chain may be a TCR β or δ chain. Preferred is the combination of an αβ or γδ TCR.

The TCR, or the antigen binding fragment thereof, is in some embodiments composed of a TCR α and a TCR β chain, or γ and δ chain. Such a double chain TCR comprises within each chain variable regions, and the variable regions each comprise one CDR1, one CDR2 and one CDR3 sequence. The TCRs comprises the CDR1 to CDR3 sequences as comprised in the variable chain amino acid sequence of SEQ ID NOs: 4 and 10; or 16 and 22; or 28 and 34; or 40 and 46; or 52 and 58; or 64 and 70; or 76 and 82; or 88 and 94; or 100 and 106; or 112 and 118.

Some embodiments of the invention pertain to a TCR, or a fragment thereof, composed of a TCR α and a TCR β chain, wherein said TCR comprises the variable region sequences having at least 50%, 60%, 70%, 80%, 90%, 95%, 98%, 99%, or preferably 100% sequence identity to the amino acid sequence selected from the α and β chain according to SEQ ID NOs: 4 and 10; or 16 and 22; or 28 and 34; or 40 and 46; or 52 and 58; or 64 and 70; or 76 and 82; or 88 and 94; or 100 and 106; or 112 and 118.

The inventive TCRs may further comprise a constant region derived from any suitable species, such as any mammal, e.g., human, rat, monkey, rabbit, donkey, or mouse. In an embodiment of the invention, the inventive TCRs further comprise a human constant region. In some preferred embodiments, the constant region of the TCR of the invention may be slightly modified, for example, by the introduction of heterologous sequences, preferably mouse sequences, which may increase TCR expression and stability.

The inventive TCRs may further comprise modified T cell receptor (TCR) a or β chains, or heterodimers comprising the same, wherein in the variable domain of said modified a or β chain, an amino acid at position 44 according to the IMGT numbering is substituted with another suitable amino acid, in order to improve pairing of desired chains. According to one embodiment of said recombinant T cell receptor (TCR) heterodimer, in at least the a or β chain, the amino acid as present at position 44 in the variable domain is substituted by one amino acid selected from the group consisting of Q, R, D, E, K, L, W, and V.

According to another embodiment, the inventive TCRs may further include one of the preferred substitution pairs selected from the following lists:

### αQ44D/βQ44R; αQ44R/βQ44D; αQ44E/βQ44K; αQ44K/βQ44E; αQ44D/βQ44K; αQ44K/βQ44D; αQ44E/βQ44R; αQ44R/βQ44E; αQ44L/βQ44W; αQ44W/βQ44L; αQ44V/βQ44W; and αQ44W/βQ44V;

### αW44D/βQ44R; αW44R/βQ44D; αW44E/βQ44K; αW44K/βQ44E; αW44D/βQ44K; αW44K/βQ44D; αW44E/βQ44R; αW44R/βQ44E; αW44L/βQ44W; αW44/βQ44L; αW44V/βQ44W; and αW44/βQ44V;

### αH44D/βQ44R; αH44R/βQ44D; αH44E/βQ44K; αH44K/βQ44E; αH44D/βQ44K; αH44K/βQ44D; αH44E/βQ44R; αH44R/βQ44E; αH44L/βQ44W; αH44W/βQ44L; αH44V/βQ44W; and αH44W/βQ44V;

### αK44D/βQ44R; αK44R/βQ44D; αK44E/βQ44K; αK44/βQ44E; αK44D/βQ44K; αK44/βQ44D; αK44E/βQ44R; αK44R/βQ44E; αK44L/βQ44W; αK44W/βQ44L; αK44V/βQ44W; and αK44W/βQ44V;

αE44D/βQ44R; αE44R/βQ44D; αE44/βQ44K; αE44K/βQ44E; αE44D/βQ44K; αE44K/βQ44D; αE44/βQ44R; αE44R/βQ44E; αE44L/βQ44W; αE44W/βQ44L; αE44V/βQ44W; and αE44W/βQ44V;

### αQ44D/βR44; αQ44R/βR44D; αQ44E/βR44K; αQ44K/βR44E; αQ44D/βR44K; αQ44K/βR44D; αQ44E/βR44; αQ44R/βR44E; αQ44L/βR44W; αQ44W/βR44L; αQ44V/βR44W; and αQ44W/βR44V;

### αW44D/βR44; αW44R/βR44D; αW44E/βR44K; αW44K/βR44E; αW44D/βR44K; αW44K/βR44D; αW44E/βR44; αW44R/βR44E; αW44L/βR44W; αW44/βR44L; αW44V/βR44W; and αW44/βR44V;

### αH44D/βR44; αH44R/βR44D; αH44E/βR44K; αH44K/βR44E; αH44D/βR44K; αH44K/βR44D; αH44E/βR44; αH44R/βR44E; αH44L/βR44W; αH44W/βR44L; αH44V/βR44W; and αH44W/βR44V;

### αK44D/βR44; αK44R/βR44D; αK44E/βR44K; αK44/βR44E; αK44D/βR44K; αK44/βR44D; αK44E/βR44; αK44R/βR44E; αK44L/βR44W; αK44W/βR44L; αK44V/βR44W; and αK44W/βR44V;

### αE44D/βR44; αE44R/βR44D; αE44/βR44K; αE44K/βR44E; αE44D/βR44K; αE44K/βR44D; αE44R/βR44E; αE44L/βR44W; αE44W/βR44L; αE44V/βR44W; and αE44W/βR44V.

In the above, e.g. “αQ44R/βQ44D” shall mean, for example, that, in the variable domain of the α chain, Q44 is substituted by R, while in the variable domain of the β chain, Q44 is substituted by D.

Some embodiments of the invention pertain to a TCR, or a fragment thereof, composed of a TCR α and a TCR β chain, wherein said TCR comprises the constant region having at least 50%, 60%, 70%, 80%, 90%, 95%, 98%, 99%, or preferably 100% sequence identity to an amino acid sequence selected from of the α and β chain according to SEQ ID NOs: 5 and 11; or 17 and 23; or 29 and 35; or 41 and 47; or 53 and 59; or 65 and 71; or 77 and 83; or 89 and 95; or 101 and 107; or 113 and 119.

The TCR α or γ chain of the invention may further comprise a CDR1 having at least 50%, 60%, 70%, 80%, 90%, 95%, 98%, 99%, or 100% sequence identity to an amino acid sequence selected from SEQ ID NOs: 1, 13, 25, 37, 49, 61, 73, 85, 97, and 109; and/or a CDR2 having at least 50%, 60%, 70%, 80%, 90%, 95%, 98%, 99%, or 100% sequence identity to an amino acid sequence selected from SEQ ID NOs: 2, 14, 26, 38, 50, 62, 74, 86, 98, and 110.

According to the invention the TCR β or δ chain may further comprise a CDR1 having at least 50%, 60%, 70%, 80%, 90%, 95%, 98%, 99%, or 100% sequence identity to an amino acid sequence selected from SEQ ID NOs: 7, 19, 31, 43, 55, 67, 79, 91, 103, and 115; and/or a CDR2 having at least 50%, 60%, 70%, 80%, 90%, 95%, 98%, 99%, or 100% sequence identity to an amino acid sequence selected from SEQ ID NOs: 8, 20, 32, 44, 56, 68, 80, 92, 104, and 116.

The antigen recognizing construct may in a further embodiment comprise a binding fragment of a TCR, and wherein said binding fragment comprises in one chain CDR1 to CDR3, optionally selected from the CDR1 to CDR3 sequences having the amino acid sequences of SEQ ID NOs: 1, 2, 3; or 7, 8, 9; or 13, 14, 15; or 19, 20, 21; or 25, 26, 27; or 31, 32, 33; or 37, 38, 39; or 43, 44, 45; or 49, 50, 51; or 55, 56, 57; or 61, 62, 63; or 67, 68, 69; or 73, 74, 75; or 79, 80, 81; or 85, 86, 87; or 91, 92, 93; or 97, 98, 99; or 103, 104, 105; or 109, 110, 111; or 115, 116, 117.

In further embodiments of the invention the antigen recognizing construct as described herein elsewhere is a TCR, or a fragment thereof, composed of at least one TCR α and one TCR β chain sequence, wherein said TCR α chain sequence comprises the CDR1 to CDR3 sequences having the amino acid sequences of SEQ ID NOs: 1 to 3, and said TCR β chain sequence comprises the CDR1 to CDR3 sequences having the amino acid sequences of SEQ ID NOs: 7 to 9; or wherein said TCR α chain sequence comprises the CDR1 to CDR3 sequences having the amino acid sequences of SEQ ID NOs: 13 to 15, and said TCR β chain sequence comprises the CDR1 to CDR3 sequences having the amino acid sequences of SEQ ID NOs: 19 to 21; or wherein said TCR α chain sequence comprises the CDR1 to CDR3 sequences having the amino acid sequences of SEQ ID NOs: 25 to 27, and said TCR β chain sequence comprises the CDR1 to CDR3 sequences having the amino acid sequences of SEQ ID NOs: 31 to 33; or wherein said TCR α chain sequence comprises the CDR1 to CDR3 sequences having the amino acid sequences of SEQ ID NOs: 37 to 39, and said TCR β chain sequence comprises the CDR1 to CDR3 sequences having the amino acid sequences of SEQ ID NOS: 43 to 45; or wherein said TCR α chain sequence comprises the CDR1 to CDR3 sequences having the amino acid sequences of SEQ ID NOs: 49 to 51, and said TCR β chain sequence comprises the CDR1 to CDR3 sequences having the amino acid sequences of SEQ ID NOs: 55 to 57; or wherein said TCR α chain sequence comprises the CDR1 to CDR3 sequences having the amino acid sequences of SEQ ID NOs: 61 to 63, and said TCR β chain sequence comprises the CDR1 to CDR3 sequences having the amino acid sequences of SEQ ID NOs: 67 to 69; or wherein said TCR α chain sequence comprises the CDR1 to CDR3 sequences having the amino acid sequences of SEQ ID NOs: 73 to 75, and said TCR β chain sequence comprises the CDR1 to CDR3 sequences having the amino acid sequences of SEQ ID NOs: 79 to 81; or wherein said TCR α chain sequence comprises the CDR1 to CDR3 sequences having the amino acid sequences of SEQ ID NOs: 85 to 87, and said TCR β chain sequence comprises the CDR1 to CDR3 sequences having the amino acid sequences of SEQ ID NOs: 91 to 93; or wherein said TCR α chain sequence comprises the CDR1 to CDR3 sequences having the amino acid sequences of SEQ ID NOs: 97 to 99, and said TCR β chain sequence comprises the CDR1 to CDR3 sequences having the amino acid sequences of SEQ ID NOs: 103 to 105; or wherein said TCR α chain sequence comprises the CDR1 to CDR3 sequences having the amino acid sequences of SEQ ID NOs: 109 to 111, and said TCR β chain sequence comprises the CDR1 to CDR3 sequences having the amino acid sequences of SEQ ID NOs: 115 to 117.

In further embodiments of the invention the antigen recognizing construct as described herein before is a TCR, or a fragment thereof, comprising at least one TCR α and one TCR β chain sequence, wherein said TCR α chain sequence comprises a variable region sequence having the amino acid sequence of SEQ ID NO: 4, and wherein said TCR β chain sequence comprises a variable region sequence having the amino acid sequence of SEQ ID NO: 10; or wherein said TCR α chain sequence comprises a variable region sequence having the amino acid sequence of SEQ ID NO: 16, and wherein said TCR β chain sequence comprises a variable region sequence having the amino acid sequence of SEQ ID NO: 22; or wherein said TCR α chain sequence comprises a variable region sequence having the amino acid sequence of SEQ ID NO: 28, and wherein said TCR β chain sequence comprises a variable region sequence having the amino acid sequence of SEQ ID NO: 34; or wherein said TCR α chain sequence comprises a variable region sequence having the amino acid sequence of SEQ ID NO: 40, and wherein said TCR β chain sequence comprises a variable region sequence having the amino acid sequence of SEQ ID NO: 46; or wherein said TCR α chain sequence comprises a variable region sequence having the amino acid sequence of SEQ ID NO: 52, and wherein said TCR β chain sequence comprises a variable region sequence having the amino acid sequence of SEQ ID NO: 58; or wherein said TCR α chain sequence comprises a variable region sequence having the amino acid sequence of SEQ ID NO: 64, and wherein said TCR β chain sequence comprises a variable region sequence having the amino acid sequence of SEQ ID NO: 70; or wherein said TCR α chain sequence comprises a variable region sequence having the amino acid sequence of SEQ ID NO: 76, and wherein said TCR β chain sequence comprises a variable region sequence having the amino acid sequence of SEQ ID NO: 82; or wherein said TCR α chain sequence comprises a variable region sequence having the amino acid sequence of SEQ ID NO: 88, and wherein said TCR β chain sequence comprises a variable region sequence having the amino acid sequence of SEQ ID NO: 94; or wherein said TCR α chain sequence comprises a variable region sequence having the amino acid sequence of SEQ ID NO: 100, and wherein said TCR β chain sequence comprises a variable region sequence having the amino acid sequence of SEQ ID NO: 106; or wherein said TCR α chain sequence comprises a variable region sequence having the amino acid sequence of SEQ ID NO: 112, and wherein said TCR β chain sequence comprises a variable region sequence having the amino acid sequence of SEQ ID NO: 118.

In further embodiments of the invention the antigen recognizing construct as described herein before is a TCR, or a fragment thereof, further comprising a TCR constant region having at least 50%, 60%, 70%, 80%, 90%, 95%, 98%, 99%, or 100% sequence identity to an amino acid sequence selected from SEQ ID NOs: 5, 11, 17, 23, 29, 35, 41, 47, 53, 59, 65, 71, 77, 83, 89, 95, 101, 107, 113, and 119, preferably wherein the TCR is composed of at least one TCR α and one TCR β chain sequence, wherein the TCR α chain sequence comprises a constant region having at least 50%, 60%, 70%, 80%, 90%, 95%, 98%, 99%, or 100% sequence identity to an amino acid sequence selected from SEQ ID NOs: 5, 17, 29, 41, 53, 65, 77, 89, 101, and 113; and wherein the TCR β chain sequence comprises a constant region having at least 50%, 60%, 70%, 80%, 90%, 95%, 98%, 99%, or 100% sequence identity to an amino acid sequence selected from SEQ ID NOs: 11, 23, 35, 47, 59, 71, 83, 95, 107, and 119.

Also disclosed are antigen recognizing constructs as described herein before comprising a first TCR chain having at least 50%, 60%, 70%, 80%, 90%, 95%, 98%, 99%, or 100% sequence identity to the amino acid sequence of SEQ ID NO: 6, and a second TCR chain having at least 50%, 60%, 70%, 80%, 90%, 95%, 98%, 99%, or 100% sequence identity to the amino acid sequence of SEQ ID NO: 12. The invention also provides TCRs comprising a first TCR chain having at least 50%, 60%, 70%, 80%, 90%, 95%, 98%, 99%, or 100% sequence identity to the amino acid sequence of SEQ ID NO: 18, and a second TCR chain having at least 50%, 60%, 70%, 80%, 90%, 95%, 98%, 99%, or 100% sequence identity to the amino acid sequence of SEQ ID NO: 24. In further embodiments the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 30, and a second TCR chain having at least 50%, 60%, 70%, 80%, 90%, 95%, 98%, 99%, or 100% sequence identity to the amino acid sequence of SEQ ID NO: 36. In further embodiments the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 42, and a second TCR chain having at least 50%, 60%, 70%, 80%, 90%, 95%, 98%, 99%, or 100sequence identity to the amino acid sequence of SEQ ID NO: 48. In further embodiments the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 54, and a second TCR chain having at least 50%, 60%, 70%, 80%, 90%, 95%, 98%, 99%, or 100% sequence identity to the amino acid sequence of SEQ ID NO: 60. In further embodiments the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO:66, and a second TCR chain having at least 50%, 60%, 70%, 80%, 90%, 95%, 98%, 99%, or 100% sequence identity to the amino acid sequence of SEQ ID NO: 72. In further embodiments the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 78, and a second TCR chain having at least 50%, 60%, 70%, 80%, 90%, 95%, 98%, 99%, or 100% sequence identity to the amino acid sequence of SEQ ID NO: 84. In further embodiments the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 90, and a second TCR chain having at least 50%, 60%, 70%, 80%, 90%, 95%, 98%, 99%, or 100% sequence identity to the amino acid sequence of SEQ ID NO: 96. In further embodiments the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 102, and a second TCR chain having at least 50%, 60%, 70%, 80%, 90%, 95%, 98%, 99%, or 100% sequence identity to the amino acid sequence of SEQ ID NO: 108. In further embodiments the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 114, and a second TCR chain having at least 50%, 60%, 70%, 80%, 90%, 95%, 98%, 99%, or 100% sequence identity to the amino acid sequence of SEQ ID NO: 120.

As used herein, the term “murine” or “human,” when referring to an antigen recognizing construct, or a TCR, or any component of a TCR described herein (e.g., complementarity determining region (CDR), variable region, constant region, α chain, and/or β chain), means a TCR (or component thereof), which is derived from a mouse or a human unrearranged TCR locus, respectively.

In an embodiment of the invention, chimeric TCR are provided, wherein the TCR chains comprise sequences from multiple species. Preferably, a TCR of the invention may comprise an α chain comprising a human variable region of an α chain and, for example, a murine constant region of a murine TCR α chain.

In one embodiment, the TCR of the invention is a human TCR comprising human variable regions according to the above embodiments and human constant regions.

In some embodiments the antigen recognizing construct is murinized or humanized. These terms are used when amino acid sequences from a foreign species are introduced into a construct of the invention.

The TCR of the invention may be provided as a single chain TCR (scTCR). A scTCR according to the invention shall comprise in one polypeptide chain a full or partial alpha chain sequence and a full or partial beta chain sequence, preferably connected via a peptide linker. A scTCR can comprise a polypeptide of a variable region of a first TCR chain (e.g., an alpha chain) and a polypeptide of an entire (full-length) second TCR chain (e.g., a beta chain), or vice versa. Furthermore, the scTCR can optionally comprise one or more linkers which join the two or more polypeptides together. The linker can be, for instance, a peptide, which joins together two single chains, as described herein. Also provided is such a scTCR of the invention, which is fused to a human cytokine, such as IL-2, IL-7 or IL-15.

The antigen recognizing construct according to the invention can also be provided in the form of a multimeric complex, comprising at least two scTCR molecules, wherein said scTCR molecules are each fused to at least one biotin moiety, or other interconnecting molecule/linker, and wherein said scTCRs are interconnected by biotin-streptavidin interaction to allow the formation of said multimeric complex. Similar approaches known in the art for the generation of multimeric TCR are also possible and included in this disclosure. Also provided are multimeric complexes of a higher order, comprising more than two scTCR of the invention.

For the purposes of the present invention, a TCR is a moiety having at least one TCR alpha or gamma and/or TCR beta or delta variable domain. Generally, they comprise both a TCR alpha variable domain and a TCR beta variable domain, alternatively both a TCR gamma variable domain and a TCR delta variable domain. They may be αβ/γδ heterodimers or may be in single chain format. For use in adoptive therapy, an αβ or γδ heterodimeric TCR may, for example, be transfected as full-length chains having both cytoplasmic and transmembrane domains. If desired, an introduced disulfide bond between residues of the respective constant domains may be present.

In a preferred embodiment, the antigen recognizing construct is a human TCR, or fragment or derivative thereof. A human TCR or fragment or derivative thereof is a TCR, which comprises over 50% of the corresponding human TCR sequence. Preferably, only a small part of the TCR sequence is of artificial origin or derived from other species. It is known, however, that chimeric TCRs, e.g. derived from human origin with murine sequences in the constant domains, are advantageous. Particularly preferred are, therefore, TCRs in accordance with the present invention, which contains murine sequences in the extracellular part of their constant domains.

Thus, it is also preferred that the inventive antigen recognizing construct is able to recognize its antigen in a human leucocyte antigen (HLA) dependent manner, preferably in a HLA-A*02 dependent manner. The term “HLA dependent manner” in the context of the present invention means that the antigen recognizing construct binds to the antigen only in the event that the antigenic peptide is presented by said HLA.

The antigen recognizing construct in accordance with the invention in one embodiment preferably induces an immune response, preferably wherein the immune response is characterized by the increase in interferon (IFN) γ levels.

Also provided by the invention is a polypeptide comprising a functional portion of any of the TCRs (or functional variants thereof) described herein, for examples, of any one of the TCRs selected from R39P1C12, R39P1F5, R40P1C2, R41P3E6, R43P3G4, R44P3B3, R44P3E7, R49P2B7, R55P1G7 and R59P2A7, as provided in the example section and table 1. The term “polypeptide” as used herein includes oligopeptides and refers to a single chain of amino acids connected by one or more peptide bonds. With respect to the inventive polypeptides, the functional portion can be any portion comprising contiguous amino acids of the TCR (or functional variant thereof), of which it is a part, provided that the functional portion specifically binds to the TAA antigen, preferably as disclosed herein in Table 2, and peptides A2 to A9 (SEQ ID NOs: 134-140) and T1 to T9 (SEQ ID: 141-149). The term “functional portion” when used in reference to a TCR (or functional variant thereof) refers to any part or fragment of the TCR (or functional variant thereof) of the invention, which part or fragment retains the biological activity of the TCR (or functional variant thereof), of which it is a part (the parent TCR or parent functional variant thereof). Functional portions encompass, for example, those parts of a TCR (or functional variant thereof) that retain the ability to specifically bind to the TAA antigen (in an HLA dependent manner), or detect, treat, or prevent cancer, to a similar extent, the same extent, or to a higher extent, as the parent TCR (or functional variant thereof). In reference to the parent TCR (or functional variant thereof), the functional portion can comprise, for instance, about 10%, 25%, 30%, 50%, 68%, 80%, 90%, 95%, or more, of the parent TCR variable sequences (or functional variant thereof).

The functional portion can comprise additional amino acids at the amino or carboxy terminus of the portion, or at both termini, in which additional amino acids are not found in the amino acid sequence of the parent TCR or functional variant thereof. Desirably, the additional amino acids do not interfere with the biological function of the functional portion, e.g., specifically binding to the TAA antigens; and/or having the ability to detect cancer, treat or prevent cancer, etc. More desirably, the additional amino acids enhance the biological activity, as compared to the biological activity of the parent TCR or functional variant thereof.

The polypeptide can comprise a functional portion of either or both of the α and β chains of the TCRs or functional variant thereof of the invention, such as a functional portion comprising one of more of CDR1, CDR2, and (preferably) CDR3 of the variable region(s) of the α chain and/or β chain of a TCR or functional variant thereof of the invention. In an embodiment of the invention, the polypeptide can comprise a functional portion comprising the amino acid sequence of SEQ ID NOs: 3, 9, 15, 21, 27, 33, 39, 45, 51, 57, 63, 69, 75, 81, 87, 93, 99, 105, 111, and 117 (CDR3 of the variable regions of the TCR of the invention), or a combination thereof. In an embodiment of the invention, the inventive polypeptide can comprise, for instance, the variable region of the inventive TCR or functional variant thereof comprising a combination of the CDR regions set forth above. In this regard, the polypeptide can comprise the amino acid sequence of any of SEQ ID NOs: 4, 10, 16, 22, 28, 34, 40, 46, 52, 58, 64, 70, 76, 82, 88, 94, 100, 106, 112, and 118 (the variable regions of an α or β chain of the TCR of the invention).

In some instances, the construct of the invention may comprise one or two polypeptide chains comprising sequences according to any of the SEQ ID NOs: 1 to 120 (CDR sequences, constant and variable regions and full-length sequences), or functional fragments thereof, and further comprise(s) other amino acid sequences, e.g., an amino acid sequence encoding an immunoglobulin or a portion thereof, then the inventive protein can be a fusion protein. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide may include any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods. In some embodiments of the invention, the TCRs (and functional portions and functional variants thereof), polypeptides, and proteins of the invention may be expressed as a single protein comprising a linker peptide linking the α chain and the β chain, and linking the γ chain and the δ chain. In this regard, the TCRs (and functional variants and functional portions thereof), polypeptides, and proteins of the invention comprising the amino acid sequences of the variable regions of the TCR of the invention and may further comprise a linker peptide. The linker peptide may advantageously facilitate the expression of a recombinant TCR (including functional portions and functional variants thereof), polypeptide, and/or protein in a host cell. The linker peptide may comprise any suitable amino acid sequence. Linker sequences for single chain TCR constructs are well known in the art. Such a single chain construct may further comprise one, or two, constant domain sequences. Upon expression of the construct including the linker peptide by a host cell, the linker peptide may also be cleaved, resulting in separated α and β chains, and separated γ and δ chain.

As already mentioned above, the binding functionality of the TCR of the invention may be provided in the framework of an antibody. For example, CDR sequences of the TCR of the invention, possibly including additional 3, 2 or 1 N and/or C terminal framework residues, may be directly grafted into an antibody variable heavy/light chain sequence. The term “antibody” in its various grammatical forms is used herein to refer to immunoglobulin molecules and immunologically active portions of immunoglobulin molecules, i.e., molecules that contain an antigen-binding site or a paratope. Such molecules are also referred to as “antigen binding fragments” of immunoglobulin molecules. The invention further provides an antibody, or antigen binding portion thereof, which specifically binds to the antigens described herein. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form.

The term “antibody” includes, but is not limited to, genetically engineered or otherwise modified forms of immunoglobulins, such as intrabodies, chimeric antibodies, fully human antibodies, humanized antibodies (e.g. generated by “CDR-grafting”), antibody fragments, and heteroconjugate antibodies (e.g., bispecific antibodies, diabodies, triabodies, tetra-bodies, etc.). The term “antibody” includes cys-diabodies and minibodies. Thus, each and every embodiment provided herein in regard to “antibodies”, or “antibody like constructs” is also envisioned as, bi-specific antibodies, diabodies, scFv fragments, chimeric antibody receptor (CAR) constructs, diabody and/or minibody embodiments, unless explicitly denoted otherwise. The term “antibody” includes a polypeptide of the immunoglobulin family or a polypeptide comprising fragments of an immunoglobulin that is capable of non-covalently, reversibly, and in a specific manner binding a corresponding antigen, preferably the TAA of the invention, as disclosed herein. An exemplary antibody structural unit comprises a tetramer. In some embodiments, a full-length antibody can be composed of two identical pairs of polypeptide chains, each pair having one “light” and one “heavy” chain (connected through a disulfide bond). Antibody structure and isotypes are well known to the skilled artisan (for example from Janeway's Immunobiology, 9th edition, 2016).

The recognized immunoglobulin genes of mammals include the kappa, lambda, alpha, gamma, delta, epsilon, and mu constant region genes, as well as the myriad immunoglobulin variable region genes (for more information on immunoglobulin genes see the international Im-MunoGeneTics information system®, Lefranc M-P et al, Nucleic Acids Res. 2015 January;43 (Database issue):D413-22; and http://www.imgt.org/). For full-length chains, the light chains are classified as either kappa or lambda. For full-length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains respectively. As used in this invention, an “antibody” encompasses all variations of antibody and fragments thereof. Thus, within the scope of this concept are full length antibodies, chimeric antibodies, humanized antibodies, single chain antibodies (scFv), Fab, Fab′, and multi-meric versions of these fragments (e.g., F(ab′)2) with the same, essentially the same or similar binding specificity. In some embodiments, the anti-body binds specifically to a peptide TAA of the invention. Preferred antigen recognizing constructs according to the invention include an antibody heavy chain, preferably the variable domain thereof, or an antigen binding fragment thereof, and/or an antibody light chain, preferably the variable domain thereof, or an antigen binding fragment thereof. Similarly, disulfide-stabilized variable region fragments (dsFv) can be prepared by recombinant DNA technology, antibody fragments of the invention, however, are not limited to these exemplary types of antibody fragments. Also, the antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles). In some instances, the TCR CDR3 sequence may be slightly modified, but preferably by not more than 3 amino acid residues, preferably only two and most preferably only one amino acid position, as compared to the CDR3 sequences provided in SEQ ID NO of the CDR3 in table 1. Preferably, the antibodies comprise the CDR3, preferably all of CDR1 to CDR3 regions in the combination, as indicated for the TCR of the invention in table 1, in each case independently, optionally with not more than three or two, preferably one, amino acid substitution(s), insertion(s) and/or deletion(s) compared to these sequences.

Suitable methods of making antibodies are known in the art. For instance, standard hybridoma methods are described in, e.g., Kohler and Milstein, Eur. J. Immunol, 5, 51 1-519 (1976), Harlow and Lane (eds.), Antibodies: A Laboratory Manual, CSH Press (1988), and C. A. Janeway et al. (eds.), Immunobiology, 8 Ed., Garland Publishing, New York, NY (2011)). Alternatively, other methods, such as EBV-hybridoma methods (Haskard and Archer, J. Immunol. Methods, 74 (2), 361-67 (1984), and Roder et al, Methods Enzymol, 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266.

Some embodiments of the invention also pertain to TCRs, or functional fragments and polypeptides thereof, which are soluble TCRs. As used herein, the term “soluble T-cell receptor” refers to heterodimeric truncated variants of native TCRs, which comprise extracellular portions of the TCR α-chain and β-chain, for example linked by a disulfide bond, but which lack the transmembrane and cytosolic domains of the native protein. The terms “soluble T-cell receptor α-chain sequence and soluble T-cell receptor β-chain sequence” refer to TCR α-chain and β-chain sequences that lack the transmembrane and cytosolic domains. The sequence (amino acid or nucleic acid) of the soluble TCR α-chain and β-chains may be identical to the corresponding sequences in a native TCR or may comprise variant soluble TCR α-chain and β-chain sequences, as compared to the corresponding native TCR sequences. The term “soluble T-cell receptor” as used herein encompasses soluble TCRs with variant or non-variant soluble TCR α-chain and β-chain sequences. The variations may be in the variable or constant regions of the soluble TCR α-chain and β-chain sequences and can include, but are not limited to, amino acid deletion, insertion, substitution mutations as well as changes to the nucleic acid sequence, which do not alter the amino acid sequence. Soluble TCR of the invention in any case retain the binding functionality of their parent molecules.

The above problem is further solved by a nucleic acid encoding for an antigen recognizing construct of the invention, or any of the aforementioned protein or polypeptide constructs. The nucleic acid preferably (a) has a strand encoding for an antigen recognizing construct according to the invention; (b) has a strand complementary to the strand in (a); or (c) has a strand that hybridizes under stringent conditions with a molecule as described in (a) or (b). Stringent conditions are known to the person of skill in the art, specifically from Sambrook et al, “Molecular Cloning”. In addition to that, the nucleic acid optionally has further sequences, which are necessary for expressing the nucleic acid sequence corresponding to the protein, specifically for expression in a mammalian/human cell. The nucleic acid used can be contained in a vector suitable for allowing expression of the nucleic acid sequence corresponding to the peptide in a cell. However, the nucleic acids can also be used to transform an antigen-presenting cell, which may not be restricted to classical antigen-presenting cells, such as dendritic cells, in such a way that they themselves produce the corresponding proteins on their cellular surface.

In some embodiments, the polypeptides of the antigen recognizing constructs can be encoded by nucleic acids and expressed in vivo or in vitro. Thus, in some embodiments, a nucleic acid encoding an antigen recognizing construct is provided. In some embodiments, the nucleic acid encodes one part or monomer of an antigen recognizing construct of the invention (for example one of two chains of a TCR of the invention), and/or another nucleic acid encodes another part or monomer of an antigen recognizing construct of the invention (for example the other of two chains of the TCR). In some embodiments, the nucleic acid encodes two or more antigen recognizing construct polypeptide chains, for example, at least 2 TCR chains. Nucleic acids encoding multiple antigen recognizing construct chains can include nucleic acid cleavage sites between at least two chain sequences, can encode transcription or translation start site between two or more chains sequences, and/or can encode proteolytic target sites between two or more antigen recognizing construct chains.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and can contain a natural, non-natural or altered internucleotide linkage, such as a phosphoroamidate linkage or a phosphorothioate linkage, instead of the phosphodiester found between the nucleotides of an unmodified oligonucleotide.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acid can comprise any nucleotide sequence, which encodes any of the TCRs, polypeptides, or proteins, or functional portions or functional variants thereof described herein.

Furthermore, the invention provides a vector comprising a nucleic acid in accordance to the invention as described above. Desirably, the vector is an expression vector or a recombinant expression vector. The term “recombinant expression vector” refers in context of the present invention to a nucleic acid construct that allows for the expression of an mRNA, protein or polypeptide in a suitable host cell. The recombinant expression vector of the invention can be any suitable recombinant expression vector, and can be used to transform or transfect any suitable host. Suitable vectors include those designed for propagation and expansion or for expression or both, such as plasmids and viruses. Examples of animal expression vectors include pEUK-Cl, pMAM, and pMAMneo. Preferably, the recombinant expression vector is a viral vector, e.g., a retroviral vector. The recombinant expression vector comprises regulatory sequences, such as transcription and translation initiation and termination codons, which are specific to the type of host cell (e.g., bacterium, fungus, plant, or animal), into which the vector is to be introduced and in which the expression of the nucleic acid of the invention may be performed. Furthermore, the vector of the invention may include one or more marker genes, which allow for selection of transformed or transfected hosts. The recombinant expression vector can comprise a native or normative promoter operably linked to the nucleotide sequence encoding the constructs of the invention, or to the nucleotide sequence, which is complementary to or which hybridizes to the nucleotide sequence encoding the constructs of the invention. The selections of promoters include, e.g., strong, weak, inducible, tissue-specific and developmental-specific promoters. The promoter can be a non-viral promoter or a viral promoter. The inventive recombinant expression vectors can be designed for either transient expression, for stable expression, or for both. Also, the recombinant expression vectors can be made for constitutive expression or for inducible expression.

The invention also pertains to a host cell comprising an antigen recognizing construct in accordance with the invention. Specifically, the host cell of the invention comprises a nucleic acid, or a vector as described herein above. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood leukocyte (PBL) or a peripheral blood mononuclear cell (PBMC). More preferably, the host cell is a T cell. The T cell can be any T cell, such as a cultured T cell, e.g., a primary T cell, or a T cell from a cultured T cell line, e.g., Jurkat, SupT1, etc., or a T cell obtained from a mammal, preferably a T cell or T cell precursor from a human patient. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. More preferably, the T cell is a T cell isolated from a human. The T cell can be any type of T cell and can be of any developmental stage, including but not limited to, CD4-positive and/or CD8-positive, CD4-positive helper T cells, e.g., Th1 and Th2 cells, CD8-positive T cells (e.g., cytotoxic T cells), tumor infiltrating cells (TILs), memory T cells, naive T cells, and the like. Preferably, the T cell is a CD8-positive T cell or a CD4-positive T cell.

Preferably, the host cell of the invention is a lymphocyte, preferably, a T lymphocyte, such as a CD4-positive or CD8-positive T-cell. The host cell furthermore preferably is a tumor reactive T cell specific for TAA expressing tumor cells.

The objective of the invention is also solved by a method of manufacturing a TAA specific antigen recognizing construct, or of a TAA specific antigen recognizing construct expressing cell line, comprising
a. Providing a suitable host cell,
b. Providing a genetic construct comprising a coding sequence encoding for an antigen recognizing construct according to the herein disclosed invention,
c. Introducing into said suitable host cell said genetic construct, and
d. Expressing said genetic construct by said suitable host cell.

The method may further comprise a step of cell surface presentation of said antigen recognizing construct on said suitable host cell.

In other preferred embodiments, the genetic construct is an expression construct comprising a promoter sequence operably linked to said coding sequence.

Preferably, said antigen recognizing construct is of mammalian origin, preferably of human origin. The preferred suitable host cell for use in the method of the invention is a mammalian cell, such as a human cell, in particular a human T lymphocyte. T cells for use in the invention are described in detail herein above.

Also encompassed by the invention are embodiments, wherein said antigen recognizing construct is a modified TCR, wherein said modification is the addition of functional domains, such as a label or a therapeutically active substance. Furthermore, encompassed are TCR having alternative domains, such as an alternative membrane anchor domain instead of the endogenous transmembrane region.

Desirably, the transfection system for introducing the genetic construct into said suitable host cell is a retroviral vector system. Such systems are well known to the skilled artisan.

Also comprised by the present invention is in one embodiment the additional method step of isolation and purification of the antigen recognizing construct from the cell and, optionally, the reconstitution of the translated antigen recognizing construct-fragments in a T-cell.

In an alternative aspect of the invention a T-cell is provided obtained or obtainable by a method for the production of a T cell receptor (TCR), which is specific for tumorous cells and has high avidity as described herein above. Such a T cell is depending on the host cell used in the method of the invention, for example, a human or non-human T-cell, preferably a human TCR.

The term “isolated” as used herein in the context of a polypeptide, such as an antigen recognizing construct (an example of which could be an antibody), refers to a polypeptide that is purified from proteins or polypeptides or other contaminants that would interfere with its therapeutic, diagnostic, prophylactic, research or other use. An antigen recognizing construct according to the invention may be a recombinant, synthetic or modified (non-natural) antigen binding construct. The term “isolated” as used herein in the context of a nucleic acid or cells refers to a nucleic acid or cells that is/are purified from DNA, RNA, proteins or polypeptides or other contaminants (such as other cells) that would interfere with its therapeutic, diagnostic, prophylactic, research or other use, or it refers to a recombinant, synthetic or modified (non-natural) nucleic acid. In this context, a “recombinant” protein/polypeptide or nucleic acid is one made using recombinant techniques. Methods and techniques for the production of recombinant nucleic acids and proteins are well known in the art.

### Treatment Methods and Diseases

One further aspect of the present invention relates to the herein disclosed antigen recognizing constructs, nucleic acids, vectors, pharmaceutical compositions and/or host cell for use in medicine. The use in medicine in one preferred embodiment includes the use in the diagnosis, prevention and/or treatment of a tumor disease, such as a malignant or benign tumor disease. The tumor disease is, for example, a tumor disease characterized by the expression of the TAA, in a cancer or tumor cell of said tumor disease.

With respect to the above mentioned medical applications of the antigen recognizing constructs and other materials derived therefrom, pertaining thereto or encoding the same, in accordance of the present disclosure, the to be treated and/or to be diagnosed diseases can be any proliferative disorder, preferably characterized by the expression of the TAA or TAA epitope sequence of the invention, for example any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is cancer is cancer of the uterine cervix, oropharynx, anus, anal canal, anorectum, vagina, vulva, or penis. A particularly preferred cancer is a TAA positive cancer, including preferably lymphoma.

The constructs, proteins, TCRs antibodies, polypeptides and nucleic acids of the invention are in particular for use in immune therapy, preferably, in adoptive T cell therapy. The administration of the compounds of the invention can, for example, involve the infusion of T cells of the invention into said patient. Preferably, such T cells are autologous T cells of the patient and in vitro transduced with a nucleic acid or antigen recognizing construct of the present invention.

The inventive antigen recognizing constructs, TCRs, polypeptides, proteins (including functional variants thereof), nucleic acids, recombinant expression vectors, host cells (including populations thereof), and antibodies (including antigen binding portions thereof), all of which are collectively referred to as “inventive TCR materials” hereinafter, can be formulated into a composition, such as a pharmaceutical composition. In this regard, the invention provides a pharmaceutical composition comprising any of the antigen recognizing constructs, TCRs, polypeptides, proteins, functional portions, functional variants, nucleic acids, expression vectors, host cells (including populations thereof), and antibodies (including antigen binding portions thereof) described herein, and a pharmaceutically acceptable carrier, excipient and/or stabilizer. The inventive pharmaceutical compositions containing any of the inventive TCR materials can comprise more than one inventive TCR material, e.g., a polypeptide and a nucleic acid, or two or more different TCRs (including functional portions and functional variants thereof). Alternatively, the pharmaceutical composition can comprise an inventive TCR material in combination with another pharmaceutically active agent(s) or drug(s), such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. Preferably, the carrier is a pharmaceutically acceptable carrier. With respect to pharmaceutical compositions, the carrier can be any of those conventionally used for the particular inventive TCR material under consideration. Such pharmaceutically acceptable carriers are well-known to those skilled in the art and are readily available to the public. It is preferred that the pharmaceutically acceptable carrier be one, which has no detrimental side effects or toxicity under the conditions of use.

Thus also provided is a pharmaceutical composition, comprising any of the herein described products of the invention and TCR materials of the invention, specifically any proteins, nucleic acids or host cells. In a preferred embodiment the pharmaceutical composition is for immune therapy, preferably adoptive cell therapy.

Preferably, the inventive TCR material is administered by injection, e.g., intravenously. When the inventive TCR material is a host cell expressing the inventive TCR (or functional variant thereof), the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an embodiment, the pharmaceutically acceptable carrier is supplemented with human serum albumen.

For purposes of the invention, the amount or dose (e.g., numbers of cells when the inventive TCR material is one or more cells) of the inventive TCR material administered may be sufficient to affect, e.g., a therapeutic or prophylactic response, in the subject or animal over a reasonable time frame. For example, the dose of the inventive TCR material should be sufficient to bind to a cancer antigen, or detect, treat or prevent cancer in a period of from about 2 hours or longer, e.g., 12 to 24 or more hours, from the time of administration. In certain embodiments, the time period could be even longer. The dose will be determined by the efficacy of the particular inventive TCR material and the condition of the animal (e.g., human), as well as the body weight of the animal (e.g., human) to be treated.

It is contemplated that the inventive pharmaceutical compositions, antigen recognizing constructs, TCRs (including functional variants thereof), polypeptides, proteins, nucleic acids, recombinant expression vectors, host cells, or populations of cells can be used in methods of treating or preventing cancer, or TAA-positive premalignancy. The inventive TCRs (and functional variants thereof) are believed to bind specifically to the TAA of the invention, such that the TCR (or related inventive polypeptide or protein and functional variants thereof), when expressed by or on a cell, such as a T cell, is able to mediate an immune response against a target cell expressing the TAA of the invention, preferably presenting TAA peptides via MHC I or II on the surface of said target cell. In this regard, the invention provides a method of treating or preventing a condition, in particular cancer, in a mammal, comprising administering to the mammal any of the pharmaceutical compositions, antigen recognizing constructs, in particular TCRs (and functional variants thereof), polypeptides, or proteins described herein, any nucleic acid or recombinant ex-pression vector comprising a nucleotide sequence encoding any of the TCRs (and functional variants thereof), polypeptides, proteins described herein, or any host cell or population of cells comprising a nucleic acid or recombinant vector, which encodes any of the constructs of the invention (and functional variants thereof), polypeptides, or proteins described herein, in an amount effective to treat or prevent the condition in the mammal, wherein the condition is preferably cancer, such as a cancer expressing the TAA of the invention.

Examples of pharmaceutically acceptable carriers or diluents useful in the present invention include stabilizers such as SPGA, carbohydrates (e.g. sorbitol, mannitol, starch, sucrose, glucose, dextran), proteins such as albumin or casein, protein containing agents such as bovine serum or skimmed milk and buffers (e.g. phosphate buffer).

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of a condition in a mammal. Furthermore, the treatment or prevention provided by the inventive method can include treatment or prevention of one or more conditions or symptoms of the condition, e.g., cancer, being treated or prevented. For example, treatment or prevention can include promoting the regression of a tumor. Also, for purposes herein, “prevention” can encompass delaying the onset of the condition, or a symptom or condition thereof.

The present invention also relates to a method of treating cancer comprising administering a TCR, a nucleic acid, or a host cell of the present description in combination with at least one chemotherapeutic agent and/or radiation therapy.

Another aspect of the invention further pertains to a method for detecting a TAA protein, or a complex of MHC and the TAA protein (protein epitope of the TAA), in a (biological) sample—such as one obtained from a subject or patient-comprising contacting the sample with an antigen recognizing construct specifically binding to said TAA peptide, or to the TAA peptide/MHC complex, and detecting the binding between said antigen recognizing construct and said TAA peptide, or to the TAA peptide/MHC complex. In some embodiments, the antigen recognizing construct is a TCR or antibody, or similar constructs, or preferably the antigen recognizing construct according to the herein described invention. In some embodiments, the (biological) sample is a sample of a tumor or a cancer (such as one of those described elsewhere herein) for example a sample comprising tumor or cancer cells.

Also provided is a method of treating cancer in a subject in need thereof, comprising:
a) isolating a cell from said subject;
b) transforming the cell with at least one vector encoding an antigen recognizing construct of the present invention to produce a transformed cell;
c) expanding the transformed cell to produce a plurality of transformed cells; and
d) administering the plurality of transformed cells to said subject.

Also provided is a method of treating cancer in a subject in need thereof, comprising:
a) isolating a cell from a healthy donor;
b) transforming the cell with a vector encoding an antigen recognizing construct of the present invention to produce a transformed cell;
c) expanding the transformed cell to produce a plurality of transformed cells; and
d) administering the plurality of transformed cells to said subject.

Also provided is a method of detecting cancer in a biological sample comprising:
a) contacting the biological sample with an antigen recognizing construct of the present description;
b) detecting binding of the antigen recognizing construct to the biological sample.

In some embodiments, the method of detecting cancer is carried out in vitro, in vivo or in situ.

Also provided is a method of detecting the presence of a condition in a mammal. The method comprises (i) contacting a sample comprising one or more cells from the mammal with any of the inventive TCRs (and functional variants thereof), polypeptides, proteins, nucleic acids, recombinant expression vectors, host cells, populations of cells, antibodies, or antigen binding portions thereof, or pharmaceutical compositions described herein, thereby forming a complex, and detecting the complex, wherein detection of the complex is indicative of the presence of the condition in the mammal, wherein the condition is cancer, such as a TAA expressing malignancy.

With respect to the inventive method of detecting a condition in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive detecting method, the contacting can take place in vitro or in vivo with respect to the mammal. Preferably, the contacting is in vitro.

Also, detection of the complex can occur through any number of ways known in the art. For instance, the inventive antigen recognizing constructs (and functional variants thereof), polypeptides, proteins, nucleic acids, recombinant expression vectors, host cells, populations of cells, or antibodies or TCRs, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the above mentioned medical applications of the TCR material of the invention, the to be treated and/or diagnosed cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is cancer of the uterine cervix, oropharynx, anus, anal canal, anorectum, vagina, vulva, or penis. A particularly preferred cancer is a TAA positive cancer, such as a Spink2 positive lymphoma.

In general, the invention provides a method for treating a subject suffering from a tumor or tumor disease comprising the administration of the antigen recognizing constructs, nucleic acids, vectors, pharmaceutical compositions and/or host cell as disclosed by the present invention. Preferably the subject is a subject in need of such a treatment. The subject in preferred embodiments is a mammalian subject, preferably a human patient, suffering from a tumor or tumor disease, which is TAA-positive.

In view of the disclosure herein it will be appreciated that the invention furthermore pertains to the following items:

Item 1: An antigen recognizing construct comprising at least one complementary determining region (CDR) 3 having at least 50% sequence identity to an amino acid sequence selected from SEQ ID NOs: 3, 9, 15, 21, 27, 33, 39, 45, 51, 57, 63, 69, 75, 81, 87, 93, 99, 105, 111, and 117.

Item 2: The antigen recognizing construct according to item 1, wherein said antigen recognizing construct is capable of specifically and/or selectively binding to a TAA of the invention antigenic peptide.

Item 3: The antigen recognizing construct according to item 1 or 2, wherein the antigen recognizing construct is an antibody, or derivative or fragment thereof, or a T cell receptor (TCR), or a derivative or fragment thereof.

Item 4: The antigen recognizing construct according to any one of items 1 to 3, wherein said antigen recognizing construct binds to a human leucocyte antigen (HLA) presented TAA antigenic peptide, wherein said HLA is optionally type A2.

Item 5: The antigen recognizing construct according to any one of items 1 to 4, wherein the construct specifically and/or selectively binds to an epitope having the amino acid sequence selected from SEQ ID NOs: 133 to 158.

Item 6: The antigen recognizing construct according to any one of items 1 to 5, wherein the construct is an α/β-TCR, or fragment or derivative thereof, or the construct is a γ/δ-TCR, or a fragment or derivative thereof.

Item 7: The antigen recognizing construct according to any one of items 1 to 6, characterized in that the construct is of human origin and specifically and/or selectively recognizes a TAA antigenic peptide.

Item 8: The antigen recognizing construct according to any one of items 1 to 7, wherein said antigen recognizing construct is capable of inducing an immune response in a subject, optionally wherein the immune response is characterized by an increase in interferon (IFN) γ levels.

Item 9: The antigen recognizing construct according to any one of items 1 to 8, comprising a TCR α or γ chain; and/or a TCR β or δ chain; wherein the TCR α or γ chain comprises a CDR3 having at least 50%, 60%, 70%, 80%, 90%, 95%, 98%, 99%, or 100% sequence identity to an amino acid sequence selected from SEQ ID NOs: 3, 15, 27, 39, 51, 63, 75, 87, 99, and 111, and/or wherein the TCR β or δ chain comprises a CDR3 having at least 50%, 60%, 70%, 80%, 90%, 95%, 98%, 99%, or 100% sequence identity to an amino acid sequence selected from SEQ ID NOs: 9, 21, 33, 45, 57, 69, 81, 93, 105, and 117.

Item 10: The antigen recognizing construct according to item 9, wherein the TCR α or γ chain further comprises a CDR1 having at least 50%, 60%, 70%, 80%, 90%, 95%, 98%, 99%, or 100% sequence identity to an amino acid sequence selected from SEQ ID NOs: 1, 13, 25, 37, 49, 61, 73, 85, 97, and 109; and/or a CDR2 having at least 50%, 60%, 70%, 80%, 90%, 95%, 98%, 99%, or 100% sequence identity to an amino acid sequence selected from SEQ ID NOs: 2, 14, 26, 38, 50, 62, 74, 86, 98, and 110.

Item 11: The antigen recognizing construct according to item 9 or 10, wherein the TCR β or δ chain further comprises a CDR1 having at least 50%, 60%, 70%, 80%, 90%, 95%, 98%, 99%, or 100% sequence identity to an amino acid sequence selected from SEQ ID NOs: 7, 19, 31, 43, 55, 67, 79, 91, 103, and 115; and/or a CDR2 having at least 50%, 60%, 70%, 80%, 90%, 95%, 98%, 99%, or 100% sequence identity to an amino acid sequence selected from SEQ ID NOs: 8, 20, 32, 44, 56, 68, 80, 92, 104, and 116.

Item 12: The antigen recognizing construct according to any of items 1 to 11, comprising a TCR variable chain region having at least 50%, 60%, 70%, 80%, 90%, 95%, 98%, 99%, or 100% sequence identity to an amino acid sequence selected from SEQ ID NOs: 4, 10, 16, 22, 28, 34, 40, 46, 52, 58, 64, 70, 76, 82, 88, 94, 100, 106, 112, and 118.

Item 13: The antigen recognizing construct according to any of items 1 to 12, wherein the construct is humanized, chimerized and/or murinized.

Item 14: The antigen recognizing construct according to any of items 1 to 13, comprising a binding fragment of a TCR, and wherein said binding fragment comprises CDR1 to CDR3 optionally selected from the CDR1 to CDR3 sequences having the amino acid sequences of SEQ ID NOs: 1, 2, 3; or 7, 8, 9; or 13, 14, 15; or 19, 20, 21; or 25, 26, 27; or 31, 32, 33; or 37, 38, 39; or 43, 44, 45; or 49, 50, 51; or 55, 56, 57; or 61, 62, 63; or 67, 68, 69; or 73, 74, 75; or 79, 80, 81; or 85, 86, 87; or 91, 92, 93; or 97, 98, 99; or 103, 104, 105; or 109, 110, 111; or 115, 116, 117.

Item 15: The antigen recognizing construct according to any of items 1 to 14, wherein the construct is a TCR, or a fragment thereof, composed of at least one TCR α and one TCR β chain sequence, wherein said TCR α chain sequence comprises the CDR1 to CDR3 sequences having the amino acid sequences of SEQ ID NOS: 1 to 3, and said TCR β chain sequence comprises the CDR1 to CDR3 sequences having the amino acid sequences of SEQ ID NOs: 7 to 9; or wherein said TCR α chain sequence comprises the CDR1 to CDR3 sequences having the amino acid sequences of SEQ ID NOS: 13 to 15, and said TCR β chain sequence comprises the CDR1 to CDR3 sequences having the amino acid sequences of SEQ ID NOs: 19 to 21; or wherein said TCR α chain sequence comprises the CDR1 to CDR3 sequences having the amino acid sequences of SEQ ID NOs: 25 to 27, and said TCR β chain sequence comprises the CDR1 to CDR3 sequences having the amino acid sequences of SEQ ID NOs: 31 to 33; or wherein said TCR α chain sequence comprises the CDR1 to CDR3 sequences having the amino acid sequences of SEQ ID NOs: 37 to 39, and said TCR β chain sequence comprises the CDR1 to CDR3 sequences having the amino acid sequences of SEQ ID NOs: 43 to 45; or wherein said TCR α chain sequence comprises the CDR1 to CDR3 sequences having the amino acid sequences of SEQ ID NOs: 49 to 51, and said TCR β chain sequence comprises the CDR1 to CDR3 sequences having the amino acid sequences of SEQ ID NOs: 55 to 57; or wherein said TCR α chain sequence comprises the CDR1 to CDR3 sequences having the amino acid sequences of SEQ ID NOs: 61 to 63, and said TCR β chain sequence comprises the CDR1 to CDR3 sequences having the amino acid sequences of SEQ ID NOs: 67 to 69; or wherein said TCR α chain sequence comprises the CDR1 to CDR3 sequences having the amino acid sequences of SEQ ID NOS: 73 to 75, and said TCR β chain sequence comprises the CDR1 to CDR3 sequences having the amino acid sequences of SEQ ID NOs: 79 to 81; or wherein said TCR α chain sequence comprises the CDR1 to CDR3 sequences having the amino acid sequences of SEQ ID NOs: 85 to 87, and said TCR β chain sequence comprises the CDR1 to CDR3 sequences having the amino acid sequences of SEQ ID NOs: 91 to 93; or wherein said TCR α chain sequence comprises the CDR1 to CDR3 sequences having the amino acid sequences of SEQ ID NOs: 97 to 99, and said TCR β chain sequence comprises the CDR1 to CDR3 sequences having the amino acid sequences of SEQ ID NOs: 103 to 105; or wherein said TCR α chain sequence comprises the CDR1 to CDR3 sequences having the amino acid sequences of SEQ ID NOs: 109 to 111, and said TCR β chain sequence comprises the CDR1 to CDR3 sequences having the amino acid sequences of SEQ ID NOs: 115 to 117.

Item 16: The antigen recognizing construct according to any of items 1 to 15, wherein the construct is a TCR, or a fragment thereof, comprising at least one TCR α and one TCR β chain sequence, wherein said TCR α chain sequence comprises a variable region sequence having the amino acid sequence of SEQ ID NO: 4, and wherein said TCR β chain sequence comprises a variable region sequence having the amino acid sequence of SEQ ID NO: 10; or wherein said TCR α chain sequence comprises a variable region sequence having the amino acid sequence of SEQ ID NO: 16, and wherein said TCR β chain sequence comprises a variable region sequence having the amino acid sequence of SEQ ID NO: 22; or wherein said TCR α chain sequence comprises a variable region sequence having the amino acid sequence of SEQ ID NO: 28, and wherein said TCR β chain sequence comprises a variable region sequence having the amino acid sequence of SEQ ID NO: 34; or wherein said TCR α chain sequence comprises a variable region sequence having the amino acid sequence of SEQ ID NO: 40, and wherein said TCR β chain sequence comprises a variable region sequence having the amino acid sequence of SEQ ID NO: 46; or wherein said TCR α chain sequence comprises a variable region sequence having the amino acid sequence of SEQ ID NO: 52, and wherein said TCR β chain sequence comprises a variable region sequence having the amino acid sequence of SEQ ID NO: 58; or wherein said TCR α chain sequence comprises a variable region sequence having the amino acid sequence of SEQ ID NO: 64, and wherein said TCR β chain sequence comprises a variable region sequence having the amino acid sequence of SEQ ID NO: 70; or wherein said TCR α chain sequence comprises a variable region sequence having the amino acid sequence of SEQ ID NO: 76, and wherein said TCR β chain sequence comprises a variable region sequence having the amino acid sequence of SEQ ID NO: 82; or wherein said TCR α chain sequence comprises a variable region sequence having the amino acid sequence of SEQ ID NO: 88, and wherein said TCR β chain sequence comprises a variable region sequence having the amino acid sequence of SEQ ID NO: 94; or wherein said TCR α chain sequence comprises a variable region sequence having the amino acid sequence of SEQ ID NO: 100, and wherein said TCR β chain sequence comprises a variable region sequence having the amino acid sequence of SEQ ID NO: 106; or wherein said TCR α chain sequence comprises a variable region sequence having the amino acid sequence of SEQ ID NO: 112, and wherein said TCR β chain sequence comprises a variable region sequence having the amino acid sequence of SEQ ID NO: 118.

Item 17: The antigen recognizing construct according to any of items 1 to 16, wherein the construct is a TCR, or a fragment thereof, further comprising a TCR constant region having at least 50%, 60%, 70%, 80%, 90%, 95%, 98%, 99%, or 100% sequence identity to an amino acid sequence selected from SEQ ID NOs: 5, 11, 17, 23, 29, 35, 41, 47, 53, 59, 65, 71, 77, 83, 89, 95, 101, 107, 113, and 119, preferably wherein the TCR is composed of at least one TCR α and one TCR β chain sequence, wherein the TCR α chain sequence comprises a constant region having at least 50%, 60%, 70%, 80%, 90%, 95%, 98%, 99%, or 100% sequence identity to an amino acid sequence selected from SEQ ID NOs: 5, 17, 29, 41, 53, 65, 77, 89, 101, and 113; and wherein the TCR β chain sequence comprises a constant region having at least 50%, 60%, 70%, 80%, 90%, 95%, 98%, 99%, or 100% sequence identity to an amino acid sequence selected from SEQ ID NOs: 11, 23, 35, 47, 59, 71, 83, 95, 107, and 119.

Item 18: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO: 6, and a second TCR chain having at least 50%, 60%, 70%, 80%, 90%, 95%, 98%, 99%, or 100% sequence identity to the amino acid sequence of SEQ ID NO: 12.

Item 19: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO: 18, and a second TCR chain having at least 50%, 60%, 70%, 80%, 90%, 95%, 98%, 99%, or 100% sequence identity to the amino acid sequence of SEQ ID NO: 24.

Item 20: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO: 30, and a second TCR chain having at least 50%, 60%, 70%, 80%, 90%, 95%, 98%, 99%, or 100% sequence identity to the amino acid sequence of SEQ ID NO: 36.

Item 20b: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO: 42, and a second TCR chain having at least 50%, 60%, 70%, 80%, 90%, 95%, 98%, 99%, or 100% sequence identity to the amino acid sequence of SEQ ID NO: 48.

Item 20c: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO: 54, and a second TCR chain having at least 50%, 60%, 70%, 80%, 90%, 95%, 98%, 99%, or 100% sequence identity to the amino acid sequence of SEQ ID NO: 60.

Item 20d: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO:66, and a second TCR chain having at least 50%, 60%, 70%, 80%, 90%, 95%, 98%, 99%, or 100% sequence identity to the amino acid sequence of SEQ ID NO: 72.

Item 20e: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO: 78, and a second TCR chain having at least 50%, 60%, 70%, 80%, 90%, 95%, 98%, 99%, or 100% sequence identity to the amino acid sequence of SEQ ID NO: 84.

Item 20f: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO: 90, and a second TCR chain having at least 50%, 60%, 70%, 80%, 90%, 95%, 98%, 99%, or 100% sequence identity to the amino acid sequence of SEQ ID NO: 96.

Item 20g: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO: 102, and a second TCR chain having at least 50%, 60%, 70%, 80%, 90%, 95%, 98%, 99%, or 100% sequence identity to the amino acid sequence of SEQ ID NO: 108.

Item 20h: The antigen recognizing construct according to any of items 1 to 17, comprising a first TCR chain having at least 50%, 60%, 70%, 80%, 90%, 95%, 98%, 99%, or 100% sequence identity to the amino acid sequence of SEQ ID NO: 114, and a second TCR chain having at least 50%, 60%, 70%, 80%, 90%, 95%, 98%, 99%, or 100% sequence identity to the amino acid sequence of SEQ ID NO: 120.

Item 21: A nucleic acid encoding for an antigen recognizing construct according to any one of items 1 to 20.

Item 22: A vector comprising a nucleic acid according to item 21.

Item 23: A host cell comprising an antigen recognizing construct according to any one of items 1 to 20, or a nucleic acid according to item 21, or a vector according to item 22.

Item 24: The host cell according to item 23, wherein the cell is a lymphocyte, preferably a T lymphocyte or T lymphocyte progenitor, more preferably a CD4 or CD8 positive T-cell.

Item 25: A pharmaceutical composition comprising the antigen recognizing construct according to any of items 1 to 20, or the nucleic acid according to item 21, or the vector according to item 22, or the host cell according to item 23 or 24, and a pharmaceutical acceptable carrier, stabilizer and/or excipient.

Item 26: The antigen recognizing construct according to any one of items 1 to 20, or a nucleic acid according to item 21, or a vector according to item 22, or a host cell according to item 23 or 24, or the pharmaceutical composition according to item 25, for use in medicine.

Item 27: The antigen recognizing construct, or the nucleic acid, or the vector, or the host cell, or the pharmaceutical composition, for use according to item 26, for use in the diagnosis, prevention, and/or treatment of a proliferative disease, wherein the disease comprises a malignant or benign tumor disease.

Item 28: The antigen recognizing construct, or the nucleic acid, or the vector, or the host cell, or the pharmaceutical composition, for use according to item 27, wherein the tumor disease is characterized by the expression of TAA in a tumor cell of the tumor disease.

Item 29: The antigen recognizing construct, or the nucleic acid, or the vector, or the host cell, or the pharmaceutical composition, for use according to any one of items 26 to 28, wherein the use in medicine is a use in immune therapy optionally comprising an adoptive cell transfer, wherein the immune therapy comprises adoptive autologous or heterologous T-cell therapy.

Item 30: A method of manufacturing a TAA specific antigen recognizing construct expressing cell line, comprising
a., providing a suitable host cell,
b., providing a genetic construct comprising a coding sequence encoding the antigen recognizing construct according to any of items 1 to 20,
c., introducing into said suitable host cell said genetic construct,
d., expressing said genetic construct by said suitable host cell.

Item 31: The method according to item 30, further comprising cell surface presentation of said antigen recognizing construct.

Item 32: The method according to item 30 or 31, wherein the genetic construct is an expression construct comprising a promoter sequence operably linked to said coding sequence.

Item 33: The method according to any one of items 30 to 32, wherein said antigen recognizing construct is of mammalian origin, preferably of human origin.

Item 34: The method according to any one of items 30 to 33, wherein said suitable host cell is a mammalian cell, optionally selected from a human cell or a human T lymphocyte.

Item 35: The method according to any of items 30 to 34, wherein said antigen recognizing construct is a modified TCR, wherein said modification comprises addition of a functional domain comprising a label, or an alternative domain comprising a membrane anchor domain.

Item 36: The method according to item 35, wherein said antigen recognizing construct is an alpha/beta TCR, gamma/delta TCR, or a single chain TCR (scTCR).

Item 37: The method according to any of items 30 to 36, wherein said genetic construct is introduced into said suitable host cell by retroviral transfection.

Item 38: The method according to any of items 30 to 37, further comprising the isolation and purification of the antigen recognizing construct from the suitable host cell and, optionally, reconstitution of the antigen recognizing construct in a T-cell.

The present invention will now be further described in the following examples with reference to the accompanying figures and sequences, nevertheless, without being limited thereto. For the purposes of the present invention, all references as cited herein are incorporated by reference in their entireties. In the Figures and Sequences:

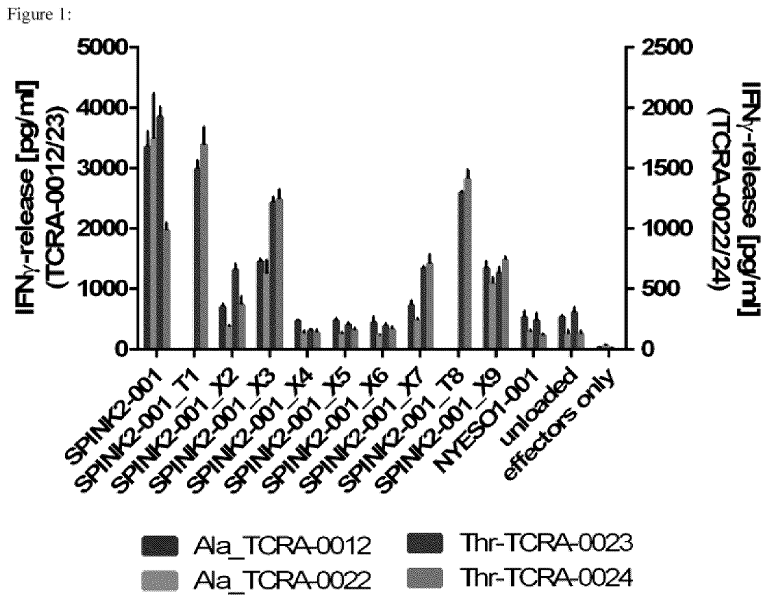

FIG. 1: IFNγ release from CD8+ T-cells electroporated with alpha and beta chain RNA of TCR R39P1C12 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOs: 134-149) or control peptide NYESO1-001 (SEQ ID NO:159). IFNy release data were obtained with CD8+ T-cells derived from two different healthy donors. RNA electroporated CD8+ T-cells alone or in co-incubation with unloaded target cells served as controls.

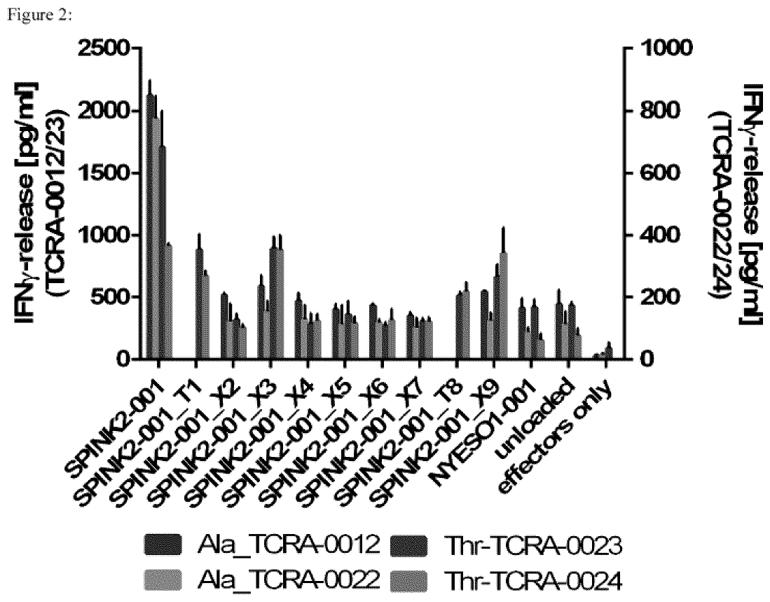

FIG. 2: IFNγ release from CD8+ T-cells electroporated with alpha and beta chain RNA of TCR R39P1F5 (Table 1) after co-incubation with T2 target cells loaded with SPINK2-001 peptide (SEQ ID NO:133) or various SPINK2-001 alanine- or threonine-substitution variants at positions 1-9 of SEQ ID NO: 133 (SEQ ID NOS: 134-149) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

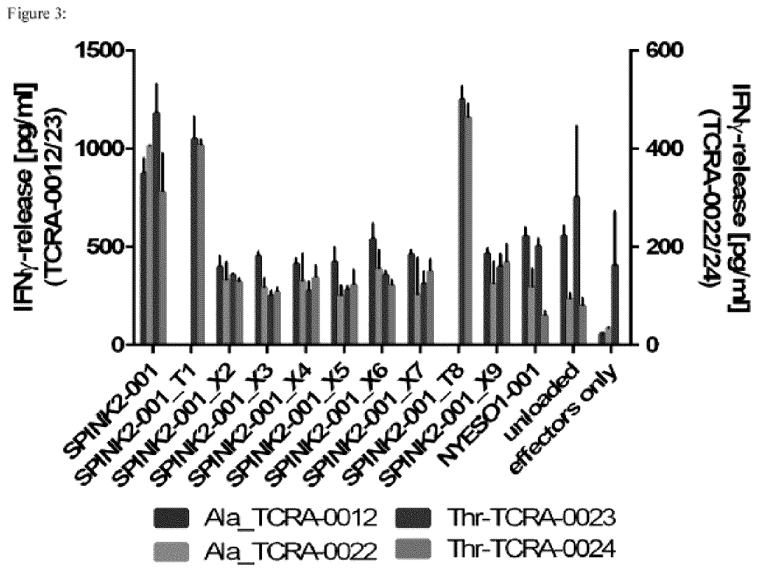

FIG. 3: IFNγ release from CD8+ T-cells electroporated with alpha and beta chain RNA of TCR R40P1C2 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOS: 134-149) or control peptide NYESO1-001 (SEQ ID NO:159). IFNy release data were obtained with CD8+ T-cells derived from two different healthy donors. RNA electroporated CD8+ T-cells alone or in co-incubation with unloaded target cells served as controls.

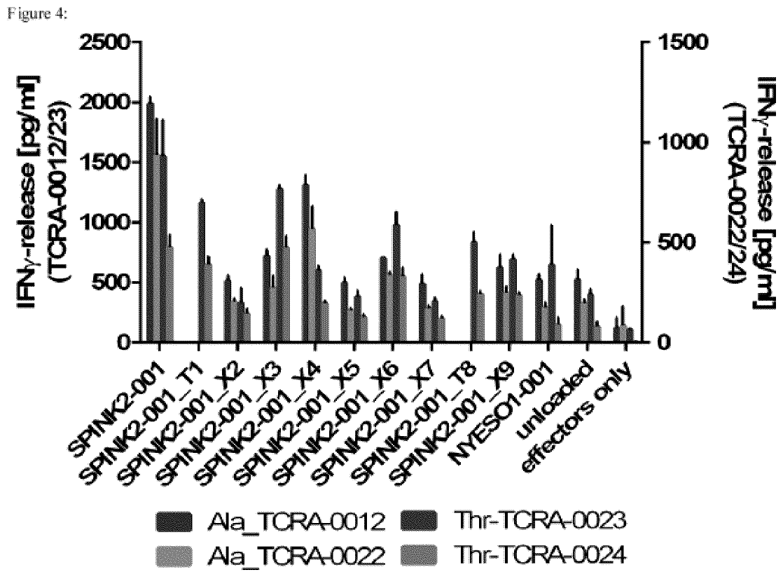

FIG. 4: IFNγ release from CD8+ T-cells electroporated with alpha and beta chain RNA of TCR R41P3E6 (Table 1) after co-incubation with T2 target cells loaded with SPINK2-001 peptide (SEQ ID NO: 133) or various SPINK2-001 alanine- or threonine-substitution variants at positions 1-9 of SEQ ID NO:133 (SEQ ID NOS: 134-149) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

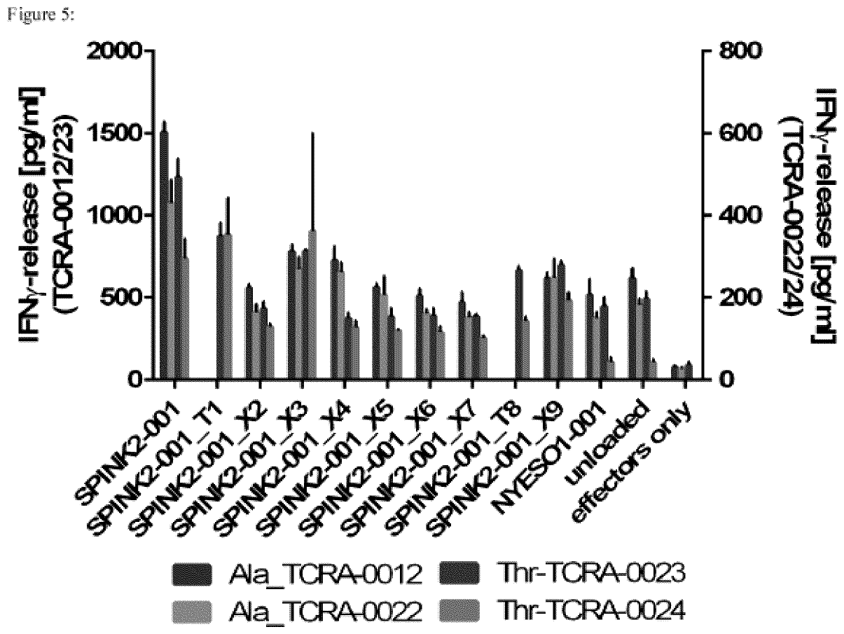

FIG. 5: IFNγ release from CD8+ T-cells electroporated with alpha and beta chain RNA of TCR R43P3G4 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOs: 134-149) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

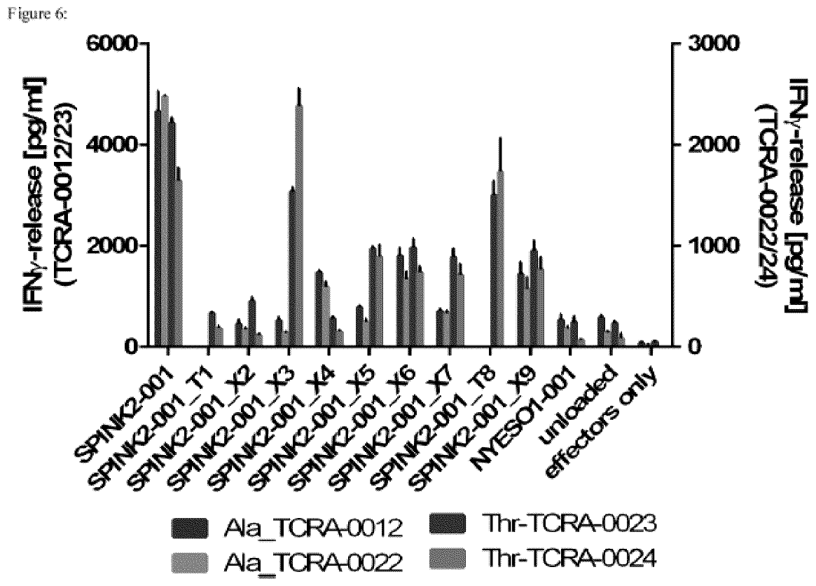

FIG. 6: IFNγ release from CD8+ T-cells electroporated with alpha and beta chain RNA of TCR R44P3B3 (Table 1) after co-incubation with T2 target cells loaded with SPINK2-001 peptide (SEQ ID NO: 133) or various SPINK2-001 alanine- or threonine-substitution variants at positions 1-9 of SEQ ID NO: 133 (SEQ ID NOs: 134-149) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

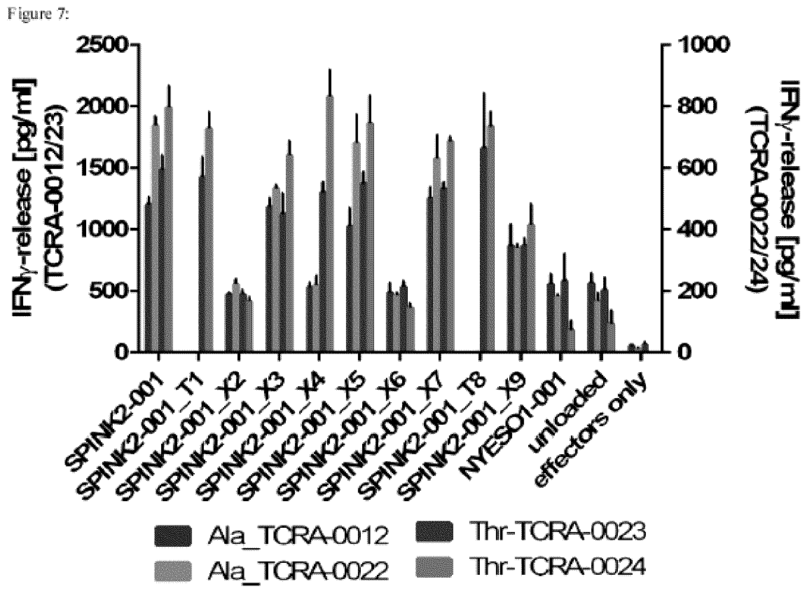

FIG. 7: IFNγ release from CD8+ T-cells electroporated with alpha and beta chain RNA of TCR R44P3E7 (Table 1) after co-incubation with T2 target cells loaded with SPINK2-001 peptide (SEQ ID NO: 133) or various SPINK2-001 alanine- or threonine-substitution variants at positions 1-9 of SEQ ID NO: 133 (SEQ ID NOs: 134-149) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

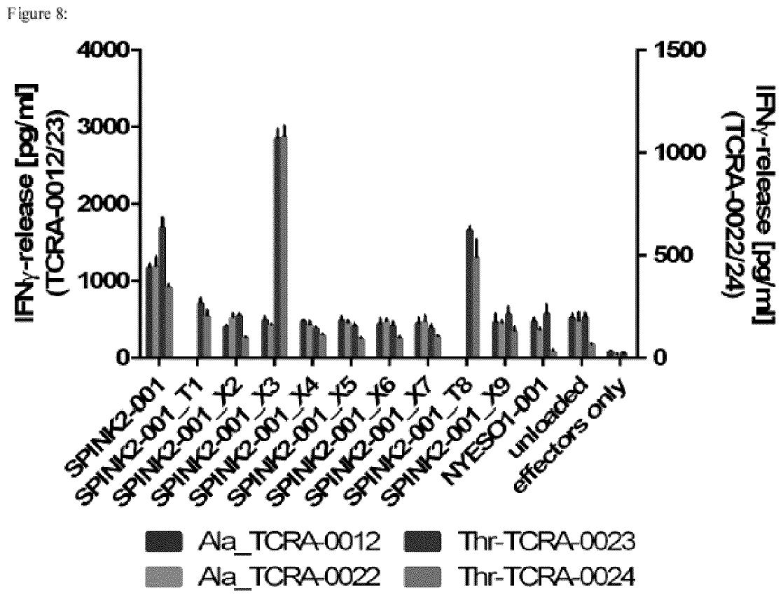

FIG. 8: IFNγ release from CD8+ T-cells electroporated with alpha and beta chain RNA of TCR R49P2B7 (Table 1) after co-incubation with T2 target cells loaded with SPINK2-001 peptide (SEQ ID NO:133) or various SPINK2-001 alanine- or threonine-substitution variants at positions 1-9 of SEQ ID NO: 133 (SEQ ID NOS: 134-149) or control peptide NYESO1-001 (SEQ ID NO:159). IFNy release data were obtained with CD8+ T-cells derived from two different healthy donors. RNA electroporated CD8+ T-cells alone or in co-incubation with unloaded target cells served as controls.

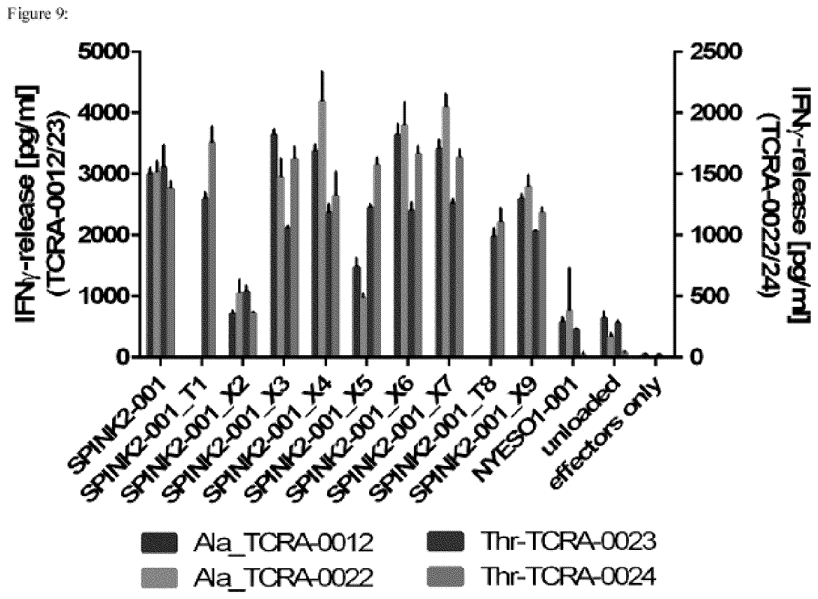

FIG. 9: IFNγ release from CD8+ T-cells electroporated with alpha and beta chain RNA of TCR R55P1G7 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOS: 134-149) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

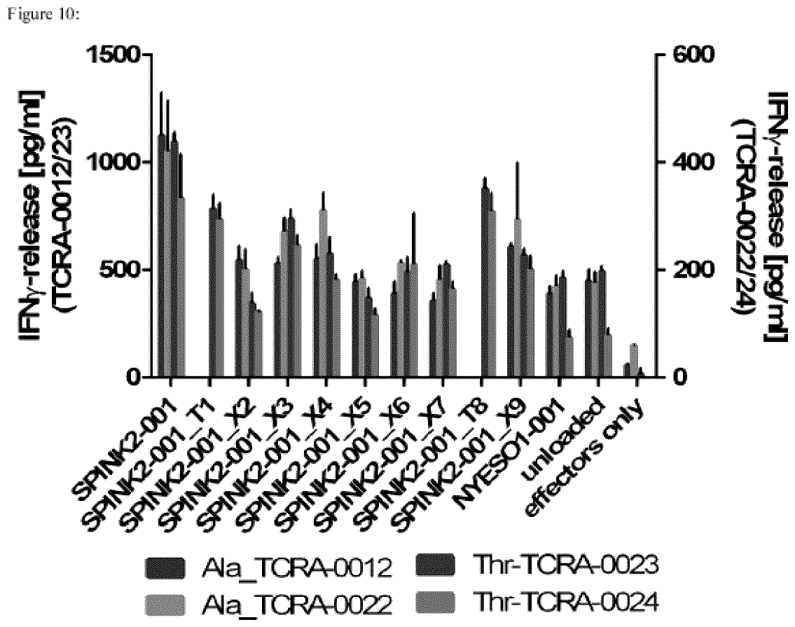

FIG. 10: IFNγ release from CD8+ T-cells electroporated with alpha and beta chain RNA of TCR R59P2A7 (Table 1) after co-incubation with T2 target cells loaded with SPINK2-001 peptide (SEQ ID NO:133) or various SPINK2-001 alanine- or threonine-substitution variants at positions 1-9 of SEQ ID NO:133 (SEQ ID NOS: 134-149) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

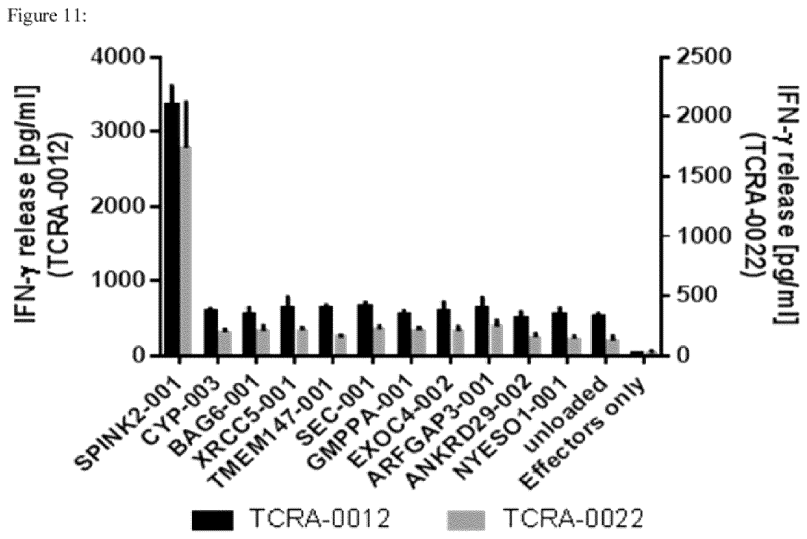

FIG. 11: IFNγ release from CD8+ T-cells electroporated with alpha and beta chain RNA of TCRs R39P1C12 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO: 158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

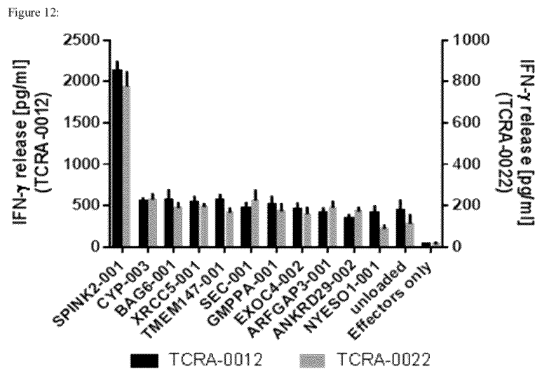

FIG. 12: IFNγ release from CD8+ T-cells electroporated with alpha and beta chain RNA of TCRs R39P1F5 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

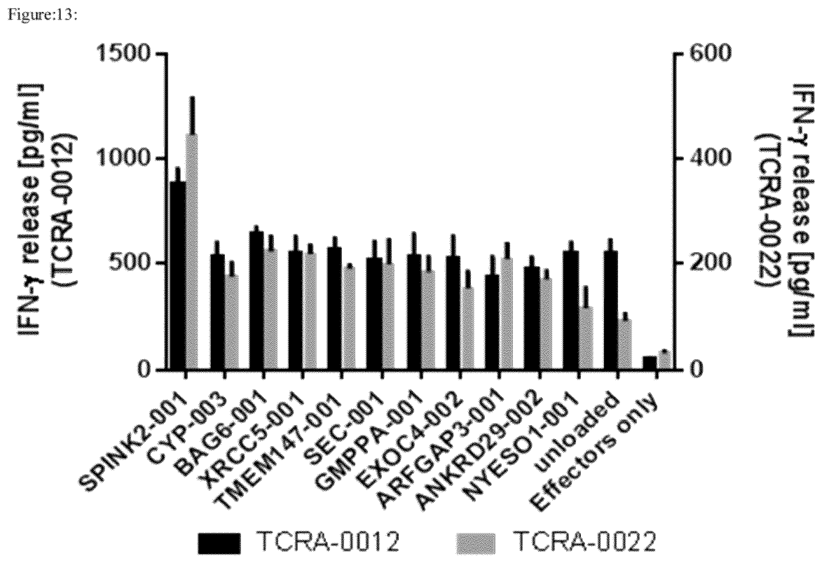

FIG. 13: IFNγ release from CD8+ T-cells electroporated with alpha and beta chain RNA of TCRs R40P1C2 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

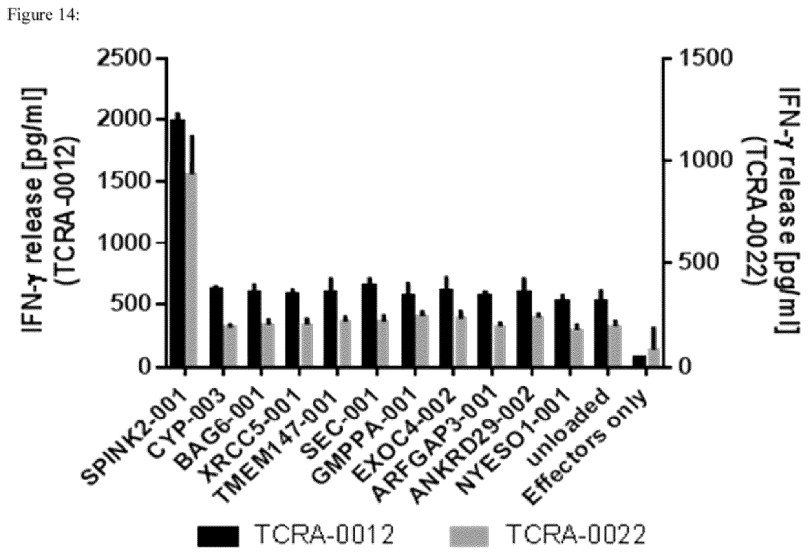

FIG. 14: IFNγ release from CD8+ T-cells electroporated with alpha and beta chain RNA of TCRs R41P3E6 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

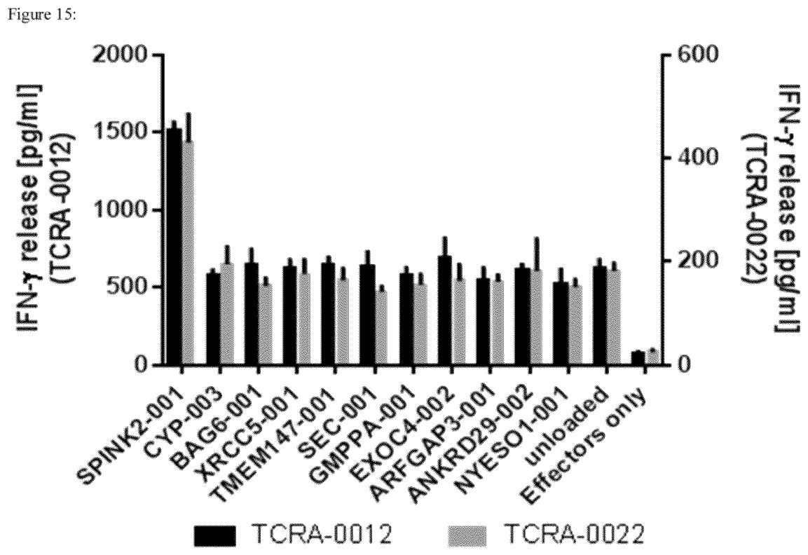

FIG. 15: IFNγ release from CD8+ T-cells electroporated with alpha and beta chain RNA of TCRs R43P3G4 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

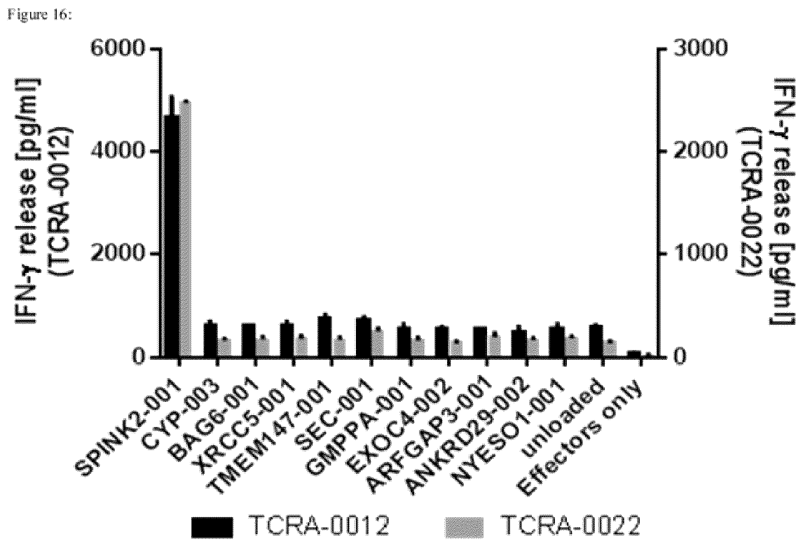

FIG. 16: IFNγ release from CD8+ T-cells electroporated with alpha and beta chain RNA of TCRs R44P3B3 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

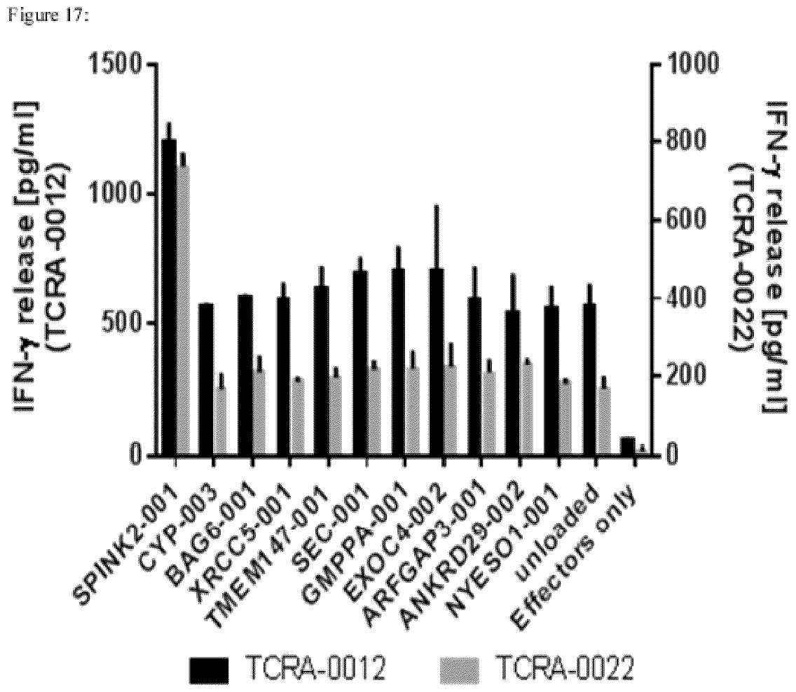

FIG. 17: IFNγ release from CD8+ T-cells electroporated with alpha and beta chain RNA of TCRs R44P3E7 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

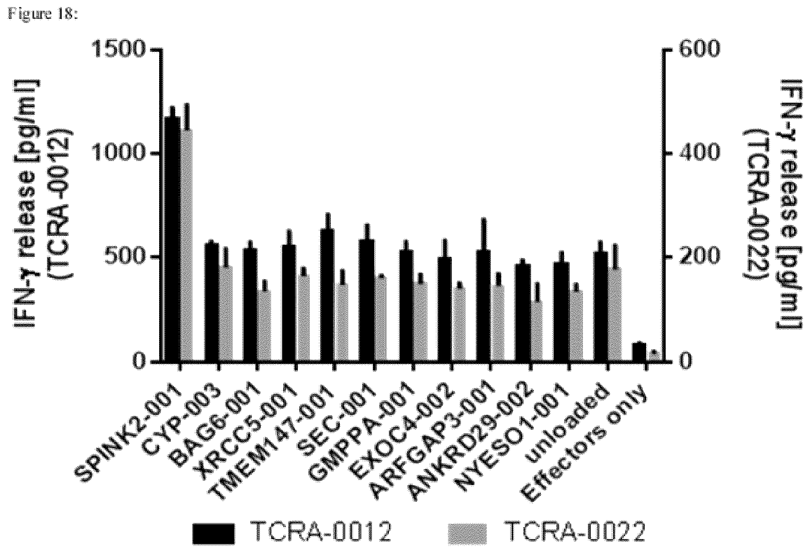

FIG. 18: IFNγ release from CD8+ T-cells electroporated with alpha and beta chain RNA of TCRs R49P2B7 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

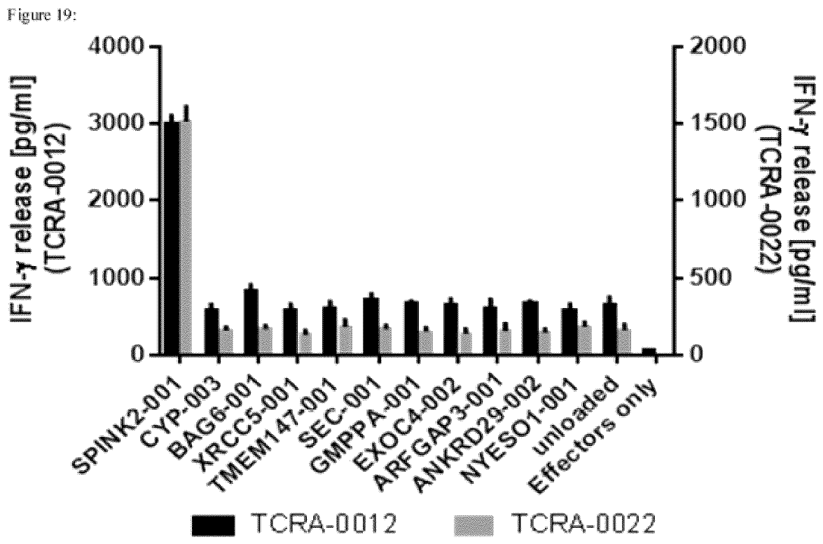

FIG. 19: IFNγ release from CD8+ T-cells electroporated with alpha and beta chain RNA of TCRs R55P1G7 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

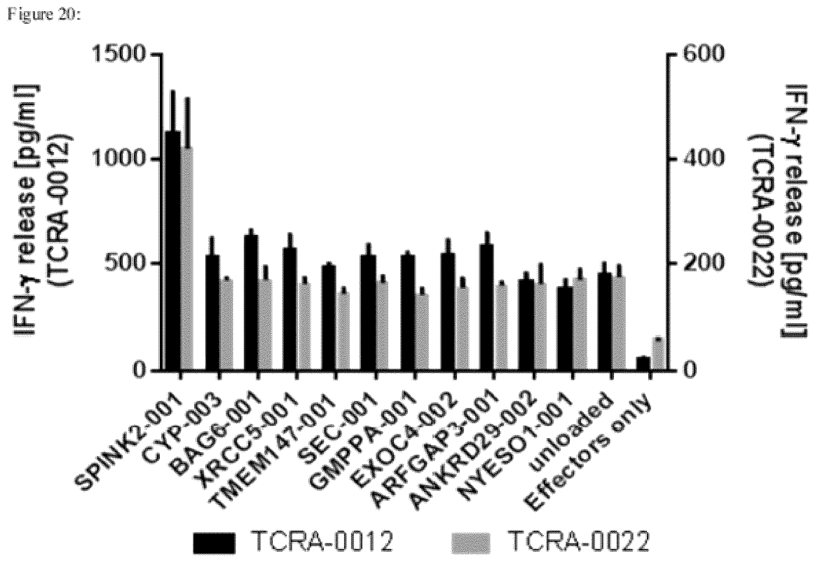

FIG. 20: IFNγ release from CD8+ T-cells electroporated with alpha and beta chain RNA of TCRs R59P2A7 (Table 1) after co-incubation with T2 target cells loaded with SPINK2-001 peptide (SEQ ID NO:133) or homologous but unrelated peptide CYP-003 (SEQ ID NO:150), BAG6-001 (SEQ ID NO:151), XRCC5-001 (SEQ ID NO:152), TMEM147-001 (SEQ ID NO:153), SEC-001 (SEQ ID NO:154), GMPPA-001 (SEQ ID NO: 155), EXOC4-002 (SEQ ID NO:156), ARFGAP3-001 (SEQ ID NO:157) or ANKRD29-002 (SEQ ID NO:158) or control peptide NYESO1-001 (SEQ ID NO:159). IFNγ release data were obtained with CD8+ T-cells derived from two different healthy donors. RNA electroporated CD8+ T-cells alone or in co-incubation with unloaded target cells served as controls.

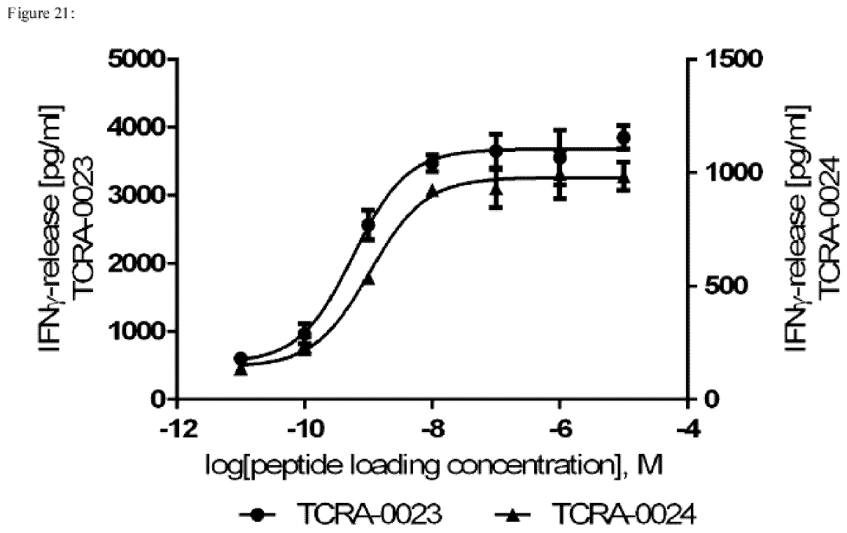

FIG. 21: IFNγ release from CD8+ T-cells electroporated with alpha and beta chain RNA of TCR R39P1C12 (Table 1) after co-incubation with T2 target cells loaded with SPINK2-001 peptide (SEQ ID NO:133) in various peptide loading concentrations from 10 μM to 10 μM. IFNγ release data were obtained with CD8+ T-cells derived from two different healthy donors.

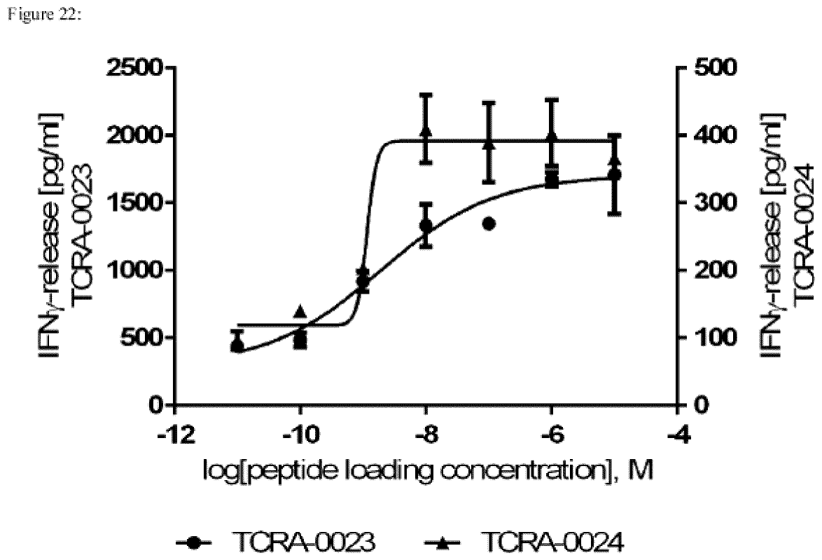

FIG. 22: IFNγ release from CD8+ T-cells electroporated with alpha and beta chain RNA of TCR R39P1F5 (Table 1) after co-incubation with T2 target cells loaded with SPINK2-001 peptide (SEQ ID NO:133) in various peptide loading concentrations from 10 μM to 10 μM. IFNγ release data were obtained with CD8+ T-cells derived from two different healthy donors.

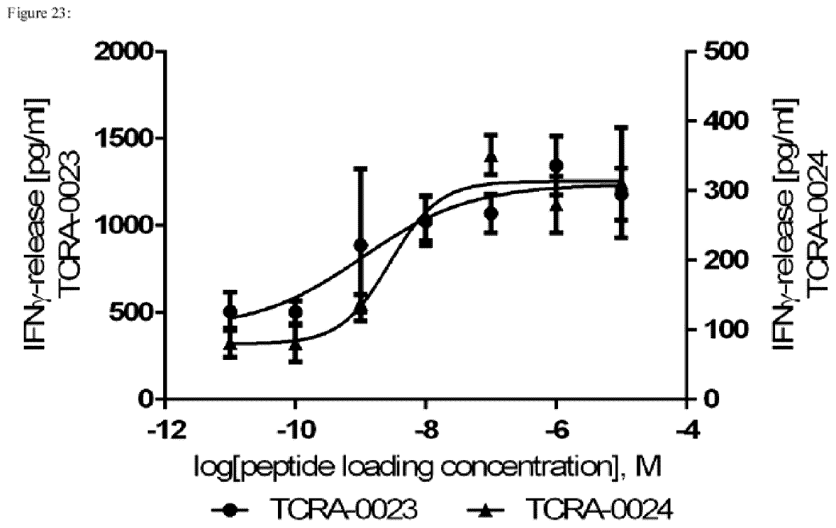

FIG. 23: IFNγ release from CD8+ T-cells electroporated with alpha and beta chain RNA of TCR R40P1C2 (Table 1) after co-incubation with T2 target cells loaded with SPINK2-001 peptide (SEQ ID NO:133) in various peptide loading concentrations from 10 μM to 10 μM. IFNγ release data were obtained with CD8+ T-cells derived from two different healthy donors.

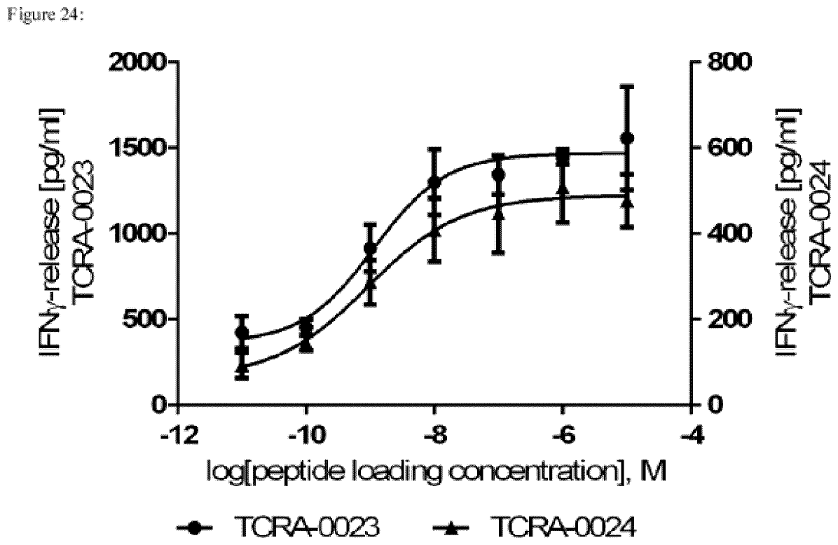

FIG. 24: IFNγ release from CD8+ T-cells electroporated with alpha and beta chain RNA of TCR R41P3E6 (Table 1) after co-incubation with T2 target cells loaded with SPINK2-001 peptide (SEQ ID NO:133) in various peptide loading concentrations from 10 μM to 10 μM. IFNγ release data were obtained with CD8+ T-cells derived from two different healthy donors.

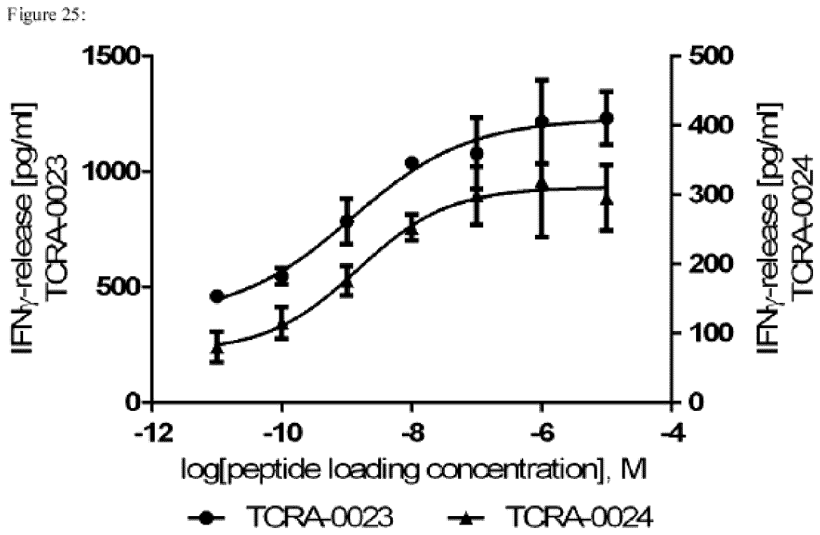

FIG. 25: IFNγ release from CD8+ T-cells electroporated with alpha and beta chain RNA of TCR R43P3G4 (Table 1) after co-incubation with T2 target cells loaded with SPINK2-001 peptide (SEQ ID NO:133) in various peptide loading concentrations from 10 μM to 10μM. IFNγ release data were obtained with CD8+ T-cells derived from two different healthy donors.

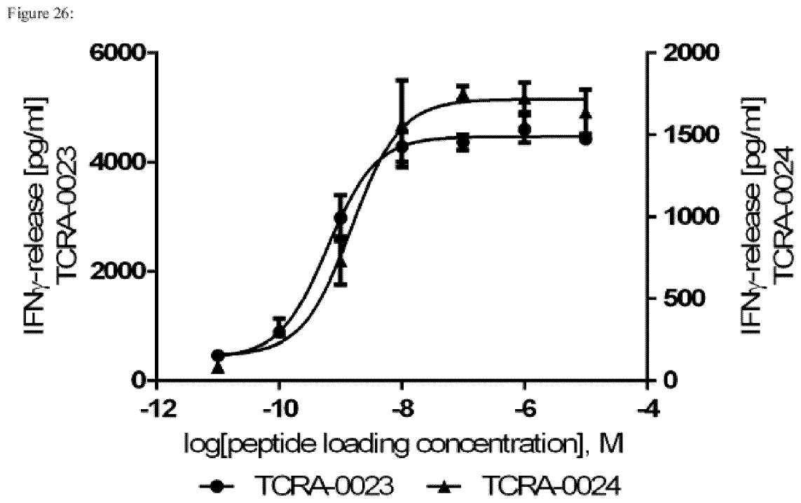

FIG. 26: IFNγ release from CD8+ T-cells electroporated with alpha and beta chain RNA of TCR R44P3B3 (Table 1) after co-incubation with T2 target cells loaded with SPINK2-001 peptide (SEQ ID NO:133) in various peptide loading concentrations from 10 μM to 10 μM. IFNγ release data were obtained with CD8+ T-cells derived from two different healthy donors.

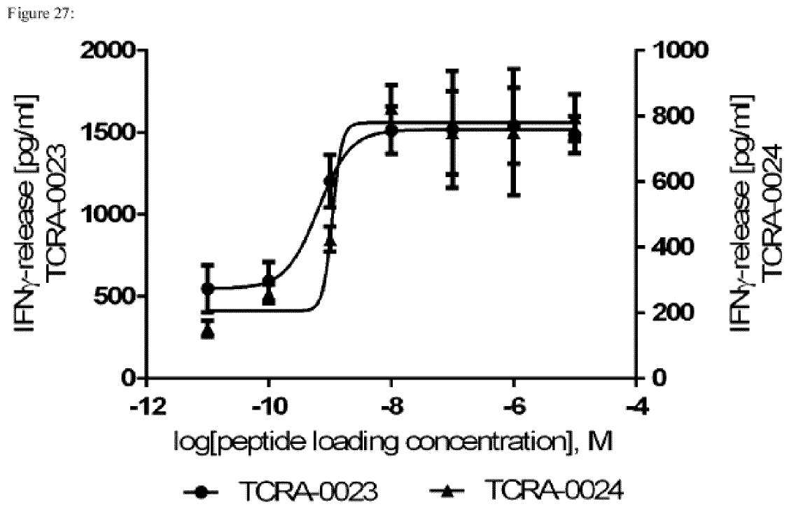

FIG. 27: IFNγ release from CD8+ T-cells electroporated with alpha and beta chain RNA of TCR R44P3E7 (Table 1) after co-incubation with T2 target cells loaded with SPINK2-001 peptide (SEQ ID NO:133) in various peptide loading concentrations from 10 μM to 10 μM. IFNγ release data were obtained with CD8+ T-cells derived from two different healthy donors.

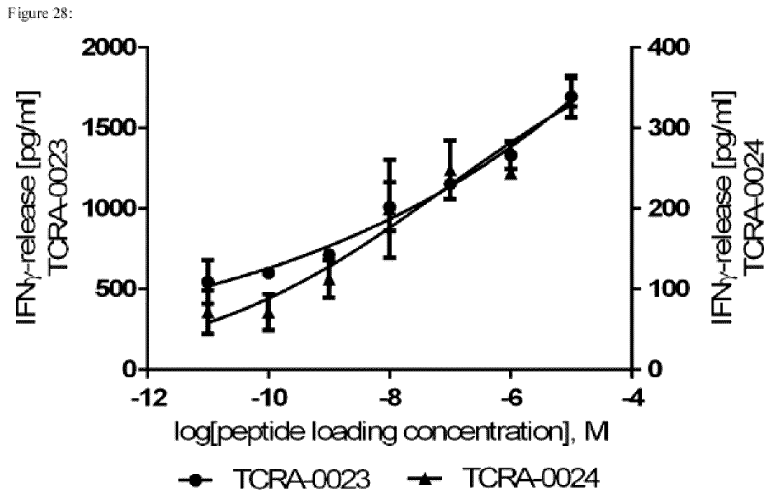

FIG. 28: IFNγ release from CD8+ T-cells electroporated with alpha and beta chain RNA of TCR R49P2B7 (Table 1) after co-incubation with T2 target cells loaded with SPINK2-001 peptide (SEQ ID NO:133) in various peptide loading concentrations from 10 μM to 10 μM. IFNγ release data were obtained with CD8+ T-cells derived from two different healthy donors.

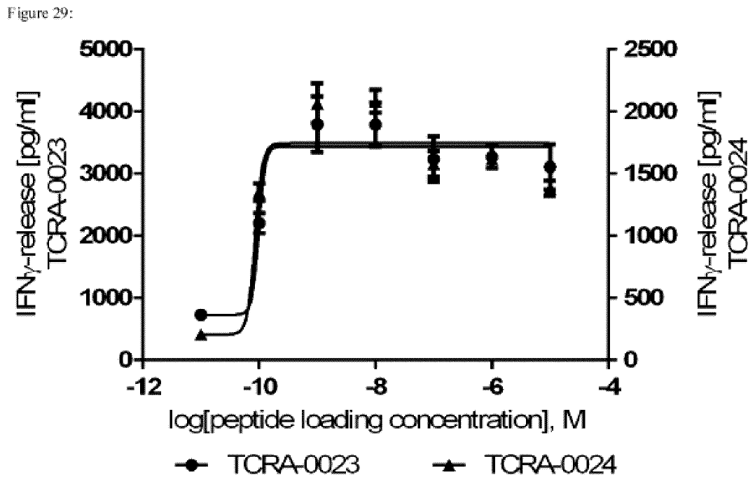

FIG. 29: IFNγ release from CD8+ T-cells electroporated with alpha and beta chain RNA of TCR R55P1G7 (Table 1) after co-incubation with T2 target cells loaded with SPINK2-001 peptide (SEQ ID NO:133) in various peptide loading concentrations from 10 μM to 10 μM. IFNγ release data were obtained with CD8+ T-cells derived from two different healthy donors.

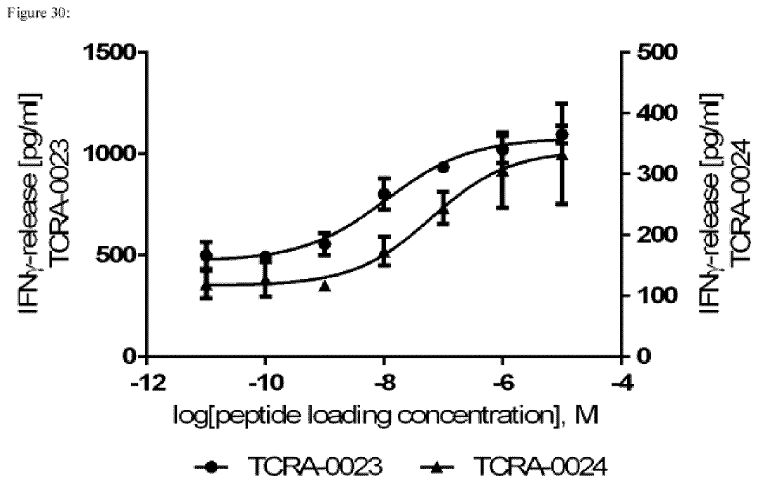

FIG. 30: IFNγ release from CD8+ T-cells electroporated with alpha and beta chain RNA of TCR R59P2A7 (Table 1) after co-incubation with T2 target cells loaded with SPINK2-001 peptide (SEQ ID NO:133) in various peptide loading concentrations from 10 μM to 10 μM. IFNγ release data were obtained with CD8+ T-cells derived from two different healthy donors.

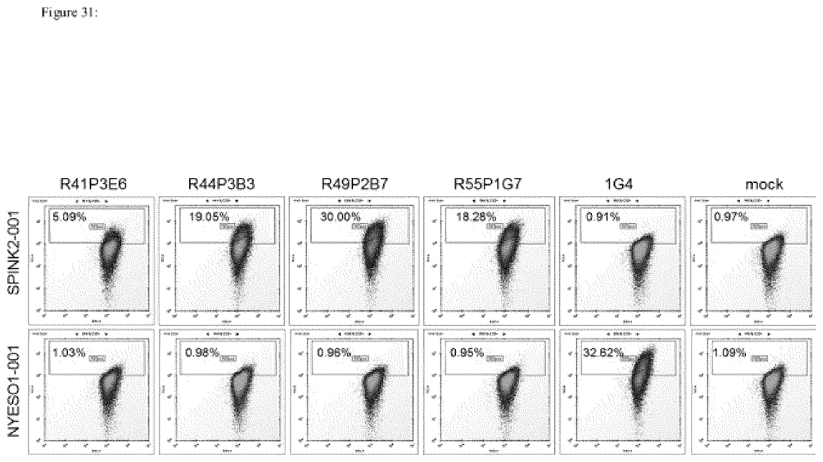

FIG. 31: HLA-A*02/SPINK2-001 tetramer or HLA-A*02/NYESO1-001 tetramer staining, respectively, of CD8+ T-cells electroporated with alpha and beta chain RNA of TCRs R41P3E6, R44P3B3, R49P2B7 and R55P1G7, respectively. CD8+ T-cells electroporated with RNA of 1G4 TCR (SEQ ID: 121-132) that specifically binds to HLA-A*02/NYESO1-001 complex and mock electroporated CD8+ T-cells served as controls.

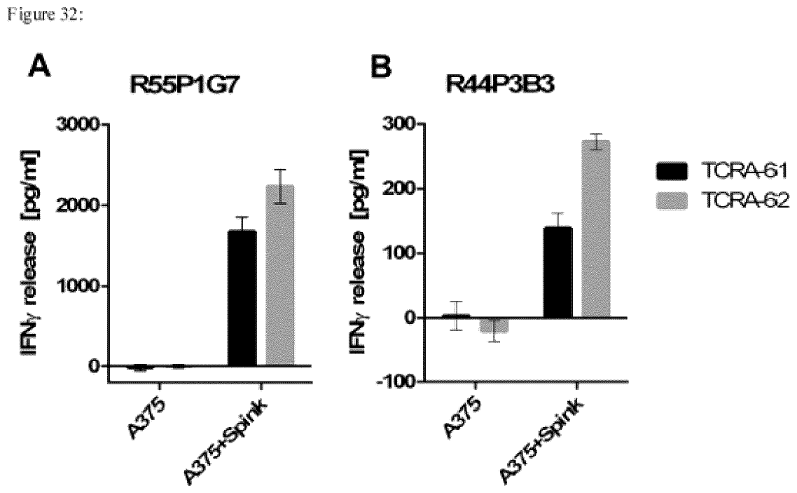

FIG. 32: IFNγ release from CD8+ T-cells electroporated with alpha and beta chain RNA of TCR R55P1G7 (A) or R44P3B3 (B) (Table 1) after co-incubation with A375 target cells overexpressing SPINK2 or wildtype A375 cells. IFNγ release data were obtained with CD8+ T-cells derived from two different healthy donors (TCRA-61 und TCRA-62). Background subtracted data is shown (i.e. pre-activation from effector only controls and signal of non-transduced effector co-cultured with target cell line was subtracted from samples of interest).

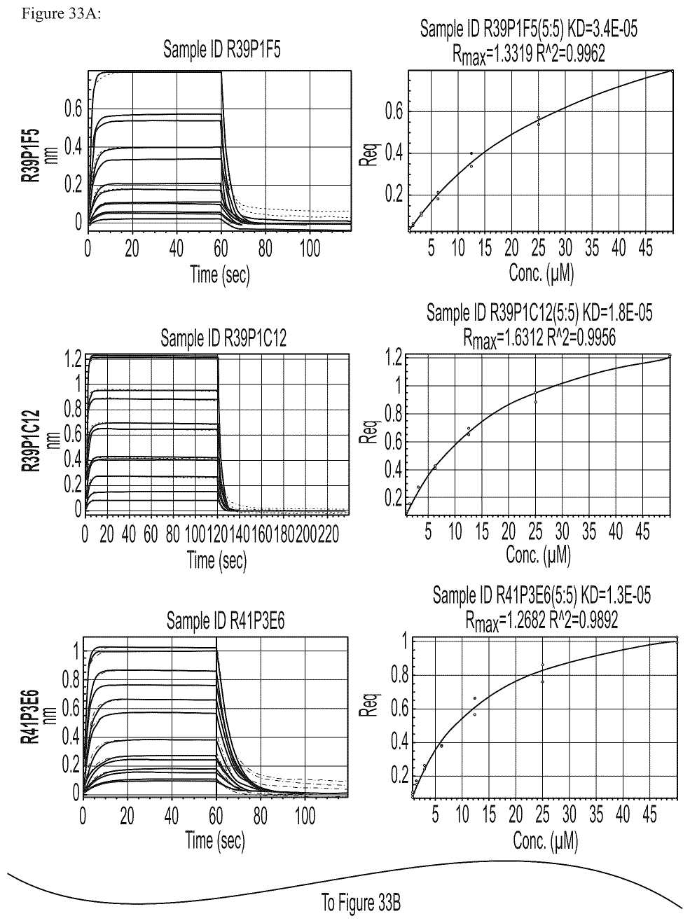

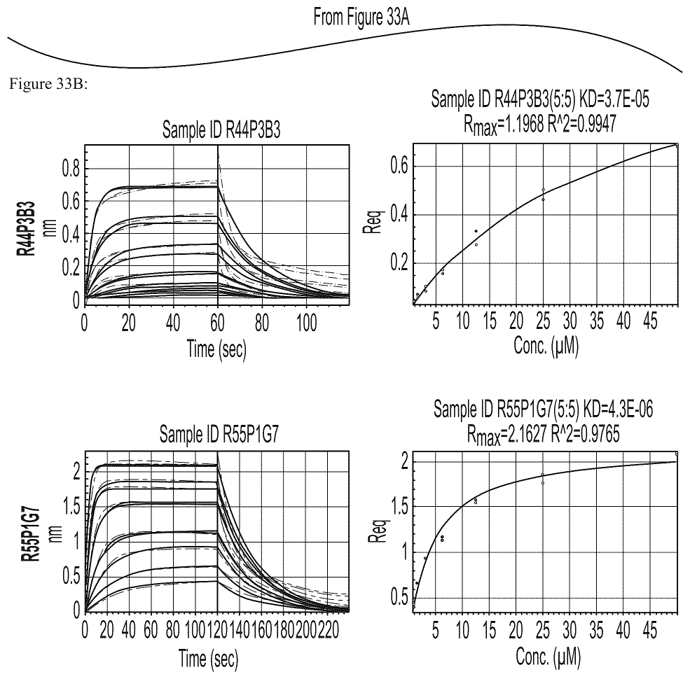

FIGS. 33A and 33B: Biolayer interferometry binding analysis of HLA-A*02:01:SPINK2-001 (immobilized on sensor) and soluble TCR variants of TCRs R39P1F5 (FIG. 33A), R39P1C12 (FIG. 33A), R41P3E6 (FIG. 33A), R44P3B3 (FIG. 33B) and R55P1G7 (FIG. 33B). Left panel shows the signal (y-axis) of binding curves and fits for different TCR concentrations over time (x-axis), right panel shows the response at the equilibrium (y-axis) over different TCR concentrations (x-axis) and fits, respectively.

TABLE 1
TCR sequences of the invention

| SEQ | | | | |
|---|---|---|---|---|
| ID NO: | TCR | Chain | Region | Sequence |
| 1 | R39P1C12 | alpha | CDR1 | DSSSTY |
| 2 | R39P1C12 | alpha | CDR2 | IFS |
| 3 | R39P1C12 | alpha | CDR3 | CAEIDNQGGKLIF |
| 4 | R39P1C12 | alpha | variable domain | MKTFAGFSFLFLWLQLDCMSRGED |
| | | | | VEQSLFLSVREGDSSVINCTYTDSSS |
| | | | | TYLYWYKQEPGAGLQLLTYIFSNMD |
| | | | | MKQDQRLTVLLNKKDKHLSLRIADT |
| | | | | QTGDSAIYFCAEIDNQGGKLIFGQGT |
| | | | | ELSVKP |
| 5 | R39P1C12 | alpha | constant domain | NIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 6 | R39P1C12 | alpha | full-length | MKTFAGFSFLFLWLQLDCMSRGED |
| | | | | VEQSLFLSVREGDSSVINCTYTDSSS |
| | | | | TYLYWYKQEPGAGLQLLTYIFSNMD |
| | | | | MKQDQRLTVLLNKKDKHLSLRIADT |
| | | | | QTGDSAIYFCAEIDNQGGKLIFGQGT |
| | | | | ELSVKPNIQNPDPAVYQLRDSKSSD |
| | | | | KSVCLFTDFDSQTNVSQSKDSDVYIT |
| | | | | DKTVLDMRSMDFKSNSAVAWSNKS |
| | | | | DFACANAFNNSIIPEDTFFPSPESSCD |
| | | | | VKLVEKSFETDTNLNFQNLSVIGFRI |
| | | | | LLLKVAGFNLLMTLRLWSS |
| 7 | R39P1C12 | beta | CDR1 | SGHDT |
| 8 | R39P1C12 | beta | CDR2 | YYEEEE |
| 9 | R39P1C12 | beta | CDR3 | CASSQLNTEAFF |
| 10 | R39P1C12 | beta | variable domain | MGPGLLCWALLCLLGAGLVDAGVT |
| | | | | QSPTHLIKTRGQQVTLRCSPKSGHDT |
| | | | | VSWYQQALGQGPQFIFQYYEEEERQ |
| | | | | RGNFPDRFSGHQFPNYSSELNVNAL |
| | | | | LLGDSALYLCASSQLNTEAFFGQGT |
| | | | | RLTVV |
| 11 | R39P1C12 | beta | constant domain | EDLNKVFPPEVAVFEPSEAEISHTQK |
| | | | | VATLVCLATGFFPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSVSYQQGVLSATILYE |
| | | | | ILLGKATLYAVLVSALVLMAMVKR |
| | | | | KDF |
| 12 | R39P1C12 | beta | full-length | MGPGLLCWALLCLLGAGLVDAGVT |
| | | | | QSPTHLIKTRGQQVTLRCSPKSGHDT |
| | | | | VSWYQQALGQGPQFIFQYYEEEERQ |
| | | | | RGNFPDRFSGHQFPNYSSELNVNAL |
| | | | | LLGDSALYLCASSQLNTEAFFGQGT |
| | | | | RLTVVEDLNKVFPPEVAVFEPSEAEI |
| | | | | SHTQKATLVCLATGFFPDHVELSW |
| | | | | WVNGKEVHSGVSTDPQPLKEQPAL |
| | | | | NDSRYCLSSRLRVSATFWQNPRNHF |
| | | | | RCQVQFYGLSENDEWTQDRAKPVT |
| | | | | QIVSAEAWGRADCGFTSVSYQQGVL |
| | | | | SATILYEILLGKATLYAVLVSALVLM |
| | | | | AMVKRKDF |
| 13 | R39P1F5 | alpha | CDR1 | DRGSQS |
| 14 | R39P1F5 | alpha | CDR2 | IY |
| 15 | R39P1F5 | alpha | CDR3 | CAVNNARLMF |
| 16 | R39P1F5 | alpha | variable domain | MKSLRVLLVILWLQLSWVWSQQKE |
| | | | | VEQNSGPLSVPEGAIASLNCTYSDRG |
| | | | | QSSFFWYRQYSGKSPELIMFIYSNGD |
| | | | | KEDGRFTAQLNKASQYVSLLIRDSQ |
| | | | | PSDSATYLCAVNNARLMFGDGTQL |
| | | | | VVKP |
| 17 | R39P1F5 | alpha | constant domain | NIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 18 | R39P1F5 | alpha | full-length | MKSLRVLLVILWLQLSWVWSQQKE |
| | | | | VEQNSGPLSVPEGAIASLNCTYSDRG |
| | | | | SQSFFWYRQYSGKSPELIMFIYSNGD |
| | | | | KEDGRFTAQLNKASQYVSLLIRDSQ |
| | | | | PSDSATYLCAVNNARLMFGDGTQL |
| | | | | VVKPNIQNPDPAVYQLRDSKSSDKS |
| | | | | VCLFTDFDSQTNVSQSKDSDVYITD |
| | | | | KTVLDMRSMDFKSNSAVAWSNKSD |
| | | | | FACANAFNNSIIPEDTFFPSPESSCDV |
| | | | | KLVEKSFETDTNLNFQNLSVIGFRIL |
| | | | | LLKVAGFNLLMTLRLWSS |
| 19 | R39P1F5 | beta | CDR1 | SNHLY |
| 20 | R39P1F5 | beta | CDR2 | FYNNEI |
| 21 | R39P1F5 | beta | CDR3 | CASSGQGANEQYF |
| 22 | R39P1F5 | beta | variable domain | MDTWLVCWAIFSLLKAGLTEPEVTQ |
| | | | | TPSHQVTQMGQEVILRCVPISNHLYF |
| | | | | YYWRQILGQKVEFLVSFYNNEISEKS |
| | | | | EIFDDQFSVERPDGSNFTLKIRSTKLE |
| | | | | DSAMYFCASSGQGANEQYFGPGTRL |
| | | | | TVT |
| 23 | R39P1F5 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 24 | R39P1F5 | beta | full-length | MDTWLVCWAIFSLLKAGLTEPEVTQ |
| | | | | TPSHQVTQMGQEVILRCVPISNHLYF |
| | | | | YWYRQILGQKVEFLVSFYNNEISEKS |
| | | | | EIFDDQFSVERPDGSNFTLKIRSTKLE |
| | | | | DSAMYFCASSGQGANEQYFGPGTRL |
| | | | | TVTEDLKNVFPPEVAVFEPSEAEISH |
| | | | | TQKATLVCLATGFYPDHVELSWWV |
| | | | | NGKEVHSGVSTDPQPLKEQPALNDS |
| | | | | RYCLSSRLRVSATFWQNPRNHFRCQ |
| | | | | VQFYGLSENDEWTQDRAKPVTQIVS |
| | | | | AEAWGRADCGFTSESYQQGVLSATI |
| | | | | LYEILLGKATLYAVLVSALVLMAM |
| | | | | VKRKDSRG |
| 25 | R40P1C2 | alpha | CDR1 | TSESDYY |
| 26 | R40P1C2 | alpha | CDR2 | QEAY |
| 27 | R40P1C2 | alpha | CDR3 | CAYLNYQLIW |
| 28 | R40P1C2 | alpha | variable domain | MACPGFLWALVISTCLEFSMAQTVT |
| | | | | QSQPEMSVQEAETVTLSCTYDTSES |
| | | | | DYYLFWYKQPPSRQMILVIRQEAYK |
| | | | | QQNATENRFSVNFQKAAKSFSLKIS |
| | | | | DSQLGDAAMYFCAYLNYQLIWGAG |
| | | | | TKLIIKP |
| 29 | R40P1C2 | alpha | constant domain | DIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 30 | R40P1C2 | alpha | full-length | MACPGFLWALVISTCLEFSMAQTVT |
| | | | | QSQPEMSVQEAETVTLSCTYDTSES |
| | | | | DYYLFWYKQPPSRQMILVIRQEAYK |
| | | | | QQNATENRFSVNFQKAAKSFSLKIS |
| | | | | DSQLGDAAMYFCAYLNYQLIWGAG |
| | | | | TKLIIKPDIQNPDPAVYQLRDSKSSD |
| | | | | KSVCLFTDFDSQTNVSQSKDSDVYIT |
| | | | | DKTVLDMRSMDFKSNSAVAWSNKS |
| | | | | DFACANAFNNSIIPEDTFFPSPESSCD |
| | | | | VKLVEKSFETDTNLNFQNLSVIGFRI |
| | | | | LLLKVAGFNLLMTLRLWSS |
| 31 | R40P1C2 | beta | CDR1 | SNHLY |
| 32 | R40P1C2 | beta | CDR2 | FYNNEI |
| 33 | R40P1C2 | beta | CDR3 | CASSEMTAVGQYF |
| 34 | R40P1C2 | beta | variable domain | MDTWLVCWAIFSLLKAGLTEPEVTQ |
| | | | | TPSHQVTQMGQEVILRCVPISNHLYF |
| | | | | YWYRQILGQKVEFLVSFYNNEISEKS |
| | | | | EIFDDQFSVERPDGSNFTLKIRSTKLE |
| | | | | DSAMYFCASSEMTAVGQYFGPGTR |
| | | | | LTVT |
| 35 | R40P1C2 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 36 | R40P1C2 | beta | full-length | MDTWLVCWAIFSLLKAGLTEPEVTQ |
| | | | | TPSHQVTQMGQEVILRCVPISNHLYF |
| | | | | YWYRQILGQKVEFLVSFYNNEISEKS |
| | | | | EIFDDQFSVERPDGSNFTLKIRSTKLE |
| | | | | DSAMYFCASSEMTAVGQYFGPGTR |
| | | | | LTVTEDLKNVFPPEVAVFEPSEAEIS |
| | | | | HTQKATLVCLATGFYPDHVELSWW |
| | | | | VNGKEVHSGVSTDPQPLKEQPALND |
| | | | | SRYCLSSRLRVSATFWQNPRNHFRC |
| | | | | QVQFYGLSENDEWTQDRAKPVTQIV |
| | | | | SAEAWGRADCGFTSESYQQGVLSAT |
| | | | | ILYEILLGKATLYAVLVSALVLMAM |
| | | | | VKRKDSRG |
| 37 | R41P3E6 | alpha | CDR1 | DRGSQS |
| 38 | R41P3E6 | alpha | CDR2 | IY |
| 39 | R41P3E6 | alpha | CDR3 | CAAFSGYALNF |
| 40 | R41P3E6 | alpha | variable domain | MKSLRVLLVILWLQLSWVWSQQKE |
| | | | | VEQNSGPLSVPEGAIASLNCTYSDRG |
| | | | | SQSFFWYRQYSGKSPELIMFIYSNGD |
| | | | | KEDGRFTAQLNKASQYVSLLIRDSQ |
| | | | | PSDSATYLCAAFSGYALNFGKGTSL |
| | | | | LVTP |
| 41 | R41P3E6 | alpha | constant domain | HIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 42 | R41P3E6 | alpha | full-length | MKSLRVLLVILWLQLSWVWSQQKE |
| | | | | VEQNSGPLSVPEGAIASLNCTYSDRG |
| | | | | SQSFFWYRQYSGKSPELIMFIYSNGD |
| | | | | KEDGRFTAQLNKASQYVSLLIRDSQ |
| | | | | PSDSATYLCAAFSGYALNFGKGTSL |
| | | | | LVTPHIQNPDPAVYQLRDSKSSDKS |
| | | | | VCLFTDFDSQTNVSQSKDSDVYITD |
| | | | | KTVLDMRSMDFKSNSAVAWSNKSD |
| | | | | FACANAFNNSIIPEDTFFPSPESSCDV |
| | | | | KLVEKSFETDTNLNFQNLSVIGFRIL |
| | | | | LLKVAGFNLLMTLRLWSS |
| 43 | R41P3E6 | beta | CDR1 | SNHLY |
| 44 | R41P3E6 | beta | CDR2 | FYNNEI |
| 45 | R41P3E6 | beta | CDR3 | CASSQYTGELFF |
| 46 | R41P3E6 | beta | variable domain | MDTWLVCWAIFSLLKAGLTEPEVTQ |
| | | | | TPSHQVTQMGQEVILRCVPISNHLYF |
| | | | | YWYRQILGQKVEFLVSFYNNEISEKS |
| | | | | EIFDDQFSVERPDGSNFTLKIRSTKLE |
| | | | | DSAMYFCASSQYTGELFFGEGSRLT |
| | | | | VL |
| 47 | R41P3E6 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 48 | R41P3E6 | beta | full-length | MDTWLVCWAIFSLLKAGLTEPEVTQ |
| | | | | TPSHQVTQMGQEVILRCVPISNHLYF |
| | | | | YWYRQILGQKVEFLVSFYNNEISEKS |
| | | | | EIFDDQFSVERPDGSNFTLKIRSTKLE |
| | | | | DSAMYFCASSQYTGELFFGEGSRLT |
| | | | | VLEDLKNVFPPEVAVFEPSEAEISHT |
| | | | | QKATLVCLATGFYPDHVELSWWVN |
| | | | | GKEVHSGVSTDPQPLKEQPALNDSR |
| | | | | YCLSSRLRVSATFWQNPRNHFRCQV |
| | | | | QFYGLSENDEWTQDRAKPVTQIVSA |
| | | | | EAWGRADCGFTSESYQQGVLSATIL |
| | | | | YEILLGKATLYAVLVSALVLMAMV |
| | | | | KRKDSRG |
| 49 | R43P3G4 | alpha | CDR1 | DRGSQS |
| 50 | R43P3G4 | alpha | CDR2 | IY |
| 51 | R43P3G4 | alpha | CDR3 | CAVNGGDMRF |
| 52 | R43P3G4 | alpha | variable domain | MKSLRVLLVILWLQLSWVWSQQKE |
| | | | | VEQNSGPLSVPEGAIASLNCTYSDRG |
| | | | | SQSFFWYRQYSGKSPELIMFIYSNGD |
| | | | | KEDGRFTAQLNKASQYVSLLIRDSQ |
| | | | | PSDSATYLCAVNGGDMRFGAGTRL |
| | | | | TVKP |
| 53 | R43P3G4 | alpha | constant domain | NIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 54 | R43P3G4 | alpha | full-length | MKSLRVLLVILWLQLSWVWSQQKE |
| | | | | VEQNSGPLSVPEGAIASLNCTYSDRG |
| | | | | SQSFFWYRQYSGKSPELIMFIYSNGD |
| | | | | KEDGRFTAQLNKASQYVSLLIRDSQ |
| | | | | PSDSATYLCAVNGGDMRFGAGTRL |
| | | | | TVKPNIQNPDPAVYQLRDSKSSDKS |
| | | | | VCLFTDFDSQTNVSQSKDSDVYITD |
| | | | | KTVLDMRSMDFKSNSAVAWSNKSD |
| | | | | FACANAFNNSIIPEDTFFPSPESSCDV |
| | | | | KLVEKSFETDTNLNFQNLSVIGFRIL |
| | | | | LLKVAGFNLLMTLRLWSS |
| 55 | R43P3G4 | beta | CDR1 | SNHLY |
| 56 | R43P3G4 | beta | CDR2 | FYNNEI |
| 57 | R43P3G4 | beta | CDR3 | CASSGQGALEQYF |
| 58 | R43P3G4 | beta | variable domain | MDTWLVCWAIFSLLKAGLTEPEVTQ |
| | | | | TPSHQVTQMGQEVILRCVPISNHLYF |
| | | | | YWYRQILGQKVEFLVSFYNNEISEKS |
| | | | | EIFDDQFSVERPDGSNFTLKIRSTKLE |
| | | | | DSAMYFCASSGQGALEQYFGPGTRL |
| | | | | TVT |
| 59 | R43P3G4 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 60 | R43P3G4 | beta | full-length | MDTWLVCWAIFSLLKAGLTEPEVTQ |
| | | | | TPSHQVTQMGQEVILRCVPISNHLYF |
| | | | | YWYRQILGQKVEFLVSFYNNEISEKS |
| | | | | EIFDDQFSVERPDGSNFTLKIRSTKLE |
| | | | | DSAMYFCASSGQGALEQYFGPGTRL |
| | | | | TVTEDLKNVFPPEVAVFEPSEAEISH |
| | | | | TQKATLVCLATGFYPDHVELSWWV |
| | | | | NGKEVHSGVSTDPQPLKEQPALNDS |
| | | | | RYCLSSRLRVSATFWQNPRNHFRCQ |
| | | | | VQFYGLSENDEWTQDRAKPVTQIVS |
| | | | | AEAWGRADCGFTSESYQQGVLSATI |
| | | | | LYEILLGKATLYAVLVSALVLMAM |
| | | | | VKRKDSRG |
| 61 | R44P3B3 | alpha | CDR1 | NSMFDY |
| 62 | R44P3B3 | alpha | CDR2 | ISS |
| 63 | R44P3B3 | alpha | CDR3 | CAASGLYNQGGKLIF |
| 64 | R44P3B3 | alpha | variable domain | MAMLLGASVLILWLQPDWVNSQQK |
| | | | | NDDQQVKQNSPSLSVQEGRISILNCD |
| | | | | YTNSMFDYFLWYKKYPAEGPTFLISI |
| | | | | SSIKDKNEDGRFTVFLNKSAKHLSLH |
| | | | | IVPSQPGDSAVYFCAASGLYNQGGK |
| | | | | LIFGQGTELSVKP |
| 65 | R44P3B3 | alpha | constant domain | NIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 66 | R44P3B3 | alpha | full-length | MAMLLGASVLILWLQPDWVNSQQK |
| | | | | NDDQQVKQNSPSLSVQEGRISILNCD |
| | | | | YTNSMFDYFLWYKKYPAEGPTFLISI |
| | | | | SSIKDKNEDGRFTVFLNKSAKHLSLH |
| | | | | IVPSQPGDSAVYFCAASGLYNQGGK |
| | | | | LIFGQGTELSVKPNIQNPDPAVYQLR |
| | | | | DSKSSDKSVCLFTDFDSQTNVSQSK |
| | | | | DSDVYITDKTVLDMRSMDFKSNSAV |
| | | | | AWSNKSDFACANAFNNSIIPEDTFFP |
| | | | | SPESSCDVKLVEKSFETDTNLNFQNL |
| | | | | SVIGFRILLLKVAGFNLLMTLRLWSS |
| 67 | R44P3B3 | beta | CDR1 | LGHDT |
| 68 | R44P3B3 | beta | CDR2 | YNNKEL |
| 69 | R44P3B3 | beta | CDR3 | CASSLGDRGYEQYF |
| 70 | R44P3B3 | beta | variable domain | MGCRLLCCVVFCLLQAGPLDTAVSQ |
| | | | | TPKYLVTQMGNDKSIKCEQNLGHDT |
| | | | | MYWYKQDSKKFLKIMFSYNNKELII |
| | | | | NETVPNRFSPKSPDKAHLNLHINSLE |
| | | | | LGDSAVYFCASSLGDRGYEQYFGPG |
| | | | | TRLTVT |
| 71 | R44P3B3 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 72 | R44P3B3 | beta | full-length | MGCRLLCCVVFCLLQAGPLDTAVSQ |
| | | | | TPKYLVTQMGNDKSIKCEQNLGHDT |
| | | | | MYWYKQDSKKFLKIMFSYNNKELII |
| | | | | NETVPNRFSPKSPDKAHLNLHINSLE |
| | | | | LGDSAVYFCASSLGDRGYEQYFGPG |
| | | | | TRLTVTEDLKNVFPPEVAVFEPSEAE |
| | | | | ISHTQKATLVCLATGFYPDHVELSW |
| | | | | WVNGKEVHSGVSTDPQPLKEQPAL |
| | | | | NDSRYCLSSRLRVSATFWQNPRNHF |
| | | | | RCQVQFYGLSENDEWTQDRAKPVT |
| | | | | QIVSAEAWGRADCGFTSESYQQGVL |
| | | | | SATILYEILLGKATLYAVLVSALVLM |
| | | | | AMVKRKDSRG |
| 73 | R44P3E7 | alpha | CDR1 | DSSSTY |
| 74 | R44P3E7 | alpha | CDR2 | IFS |
| 75 | R44P3E7 | alpha | CDR3 | CAEINNNARLMF |
| 76 | R44P3E7 | alpha | variable domain | MKTFAGFSFLFLWLQLDCMSRGED |
| | | | | VEQSLFLSVREGDSSVINCTYTDSSS |
| | | | | TYLYWYKQEPGAGLQLLTYIFSNMD |
| | | | | MKQDQRLTVLLNKKDKHLSLRIADT |
| | | | | QTGDSAIYFCAEINNNARLMFGDGT |
| | | | | QLVVKP |
| 77 | R44P3E7 | alpha | constant domain | NIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 78 | R44P3E7 | alpha | full-length | MKTFAGFSFLFLWLQLDCMSRGED |
| | | | | VEQSLFLSVREGDSSVINCTYTDSSS |
| | | | | TYLYWYKQEPGAGLQLLTYIFSNMD |
| | | | | MKQDQRLTVLLNKKDKHLSLRIADT |
| | | | | QTGDSAIYFCAEINNNARLMFGDGT |
| | | | | QLVVKPNIQNPDPAVYQLRDSKSSD |
| | | | | KSVCLFTDFDSQTNVSQSKDSDVYIT |
| | | | | DKTVLDMRSMDFKSNSAVAWSNKS |
| | | | | DFACANAFNNSIIPEDTFFPSPESSCD |
| | | | | VKLVEKSFETDTNLNFQNLSVIGFRI |
| | | | | LLLKVAGFNLLMTLRLWSS |
| 79 | R44P3E7 | beta | CDR1 | PRHDT |
| 80 | R44P3E7 | beta | CDR2 | FYEKMQ |
| 81 | R44P3E7 | beta | CDR3 | CASSPPDQNTQYF |
| 82 | R44P3E7 | beta | variable domain | MLSPDLPDSAWNTRLLCHVMLCLL |
| | | | | GAVSVAAGVIQSPRHLIKEKRETATL |
| | | | | KCYPIPRHDTVYWYQQGPGQDPQFL |
| | | | | ISFYEKMQSDKGSIPDRFSAQQFSDY |
| | | | | HSELNMSSLELGDSALYFCASSPPDQ |
| | | | | NTQYFGPGTRLTVL |
| 83 | R44P3E7 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 84 | R44P3E7 | beta | full-length | MLSPDLPDSAWNTRLLCHVMLCLL |
| | | | | GAVSVAAGVIQSPRHLIKEKRETATL |
| | | | | KCYPIPRHDTVYWYQQGPGQDPQFL |
| | | | | ISFYEKMQSDKGSIPDRFSAQQFSDY |
| | | | | HSELNMSSLELGDSALYFCASSPPDQ |
| | | | | NTQYFGPGTRLTVLEDLKNVFPPEV |
| | | | | AVFEPSEAEISHTQKATLVCLATGFY |
| | | | | PDHVELSWWVNGKEVHSGVSTDPQ |
| | | | | PLKEQPALNDSRYCLSSRLRVSATF |
| | | | | WQNPRNHFRCQVQFYGLSENDEWT |
| | | | | QDRAKPVTQIVSAEAWGRADCGFTS |
| | | | | ESYQQGVLSATILYEILLGKATLYAV |
| | | | | LVSALVLMAMVKRKDSRG |
| 85 | R49P2B7 | alpha | CDR1 | SSVPPY |
| 86 | R49P2B7 | alpha | CDR2 | YTTG |
| 87 | R49P2B7 | alpha | CDR3 | CAVRIFGNEKLTF |
| 88 | R49P2B7 | alpha | variable domain | MLLLLVPVLEVIFTLGGTRAQSVTQL |
| | | | | GSHVSVSEGALVLLRCNYSSSVPPYL |
| | | | | FWYVQYPNQGLQLLLKYTTGATLV |
| | | | | KGINGFEAEFKKSETSFHLTKPSAHM |
| | | | | SDAAEYFCAVRIFGNEKLTFGTGTRL |
| | | | | TIIP |
| 89 | R49P2B7 | alpha | constant domain | NIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 90 | R49P2B7 | alpha | full-length | MLLLLVPVLEVIFTLGGTRAQSVTQL |
| | | | | GSHVSVSEGALVLLRCNYSSSVPPYL |
| | | | | FWYVQYPNQGLQLLLKYTTGATLV |
| | | | | KGINGFEAEFKKSETSFHLTKPSAHM |
| | | | | SDAAEYFCAVRIFGNEKLTFGTGTRL |
| | | | | TIIPNIQNPDPAVYQLRDSKSSDKSV |
| | | | | CLFTDFDSQTNVSQSKDSDVYITDKT |
| | | | | VLDMRSMDFKSNSAVAWSNKSDFA |
| | | | | CANAFNNSIIPEDTFFPSPESSCDVKL |
| | | | | VEKSFETDTNLNFQNLSVIGFRILLL |
| | | | | KVAGFNLLMTLRLWSS |
| 91 | R49P2B7 | beta | CDR1 | MDHEN |
| 92 | R49P2B7 | beta | CDR2 | SYDVKM |
| 93 | R49P2B7 | beta | CDR3 | CASSLMGELTGELFF |
| 94 | R49P2B7 | beta | variable domain | MGIRLLCRVAFCFLAVGLVDVKVTQ |
| | | | | SSRYLVKRTGEKVFLECVQDMDHE |
| | | | | NMFWYRQDPGLGLRLIYFSYDVKM |
| | | | | KEKGDIPEGYSVSREKKERFSLILES |
| | | | | ASTNQTSMYLCASSLMGELTGELFF |
| | | | | GEGSRLTVL |
| 95 | R49P2B7 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 96 | R49P2B7 | beta | full-length | MGIRLLCRVAFCFLAVGLVDVKVTQ |
| | | | | SSRYLVKRTGEKVFLECVQDMDHE |
| | | | | NMFWYRQDPGLGLRLIYFSYDVKM |
| | | | | KEKGDIPEGYSVSREKKERFSLILES |
| | | | | ASTNQTSMYLCASSLMGELTGELFF |
| | | | | GEGSRLTVLEDLKNVFPPEVAVFEPS |
| | | | | EAEISHTQKATLVCLATGFYPDHVE |
| | | | | LSWWVNGKEVHSGVSTDPQPLKEQ |
| | | | | PALNDSRYCLSSRLRVSATFWQNPR |
| | | | | NHERCQVQFYGLSENDEWTQDRAK |
| | | | | PVTQIVSAEAWGRADCGFTSESYQQ |
| | | | | GVLSATILYEILLGKATLYAVLVSAL |
| | | | | VLMAMVKRKDSRG |
| 97 | R55P1G7 | alpha | CDR1 | NSAFQY |
| 98 | R55P1G7 | alpha | CDR2 | TY |
| 99 | R55P1G7 | alpha | CDR3 | CAMMGDTGTASKLTF |
| 100 | R55P1G7 | alpha | variable domain | MMKSLRVLLVILWLQLSWVWSQQK |
| | | | | EVEQDPGPLSVPEGAIVSLNCTYSNS |
| | | | | AFQYFMWYRQYSRKGPELLMYTYS |
| | | | | SGNKEDGRFTAQVDKSSKYISLFIRD |
| | | | | SQPSDSATYLCAMMGDTGTASKLTF |
| | | | | GTGTRLQVTL |
| 101 | R55P1G7 | alpha | constant domain | DIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 102 | R55P1G7 | alpha | full-length | MMKSLRVLLVILWLQLSWVWSQQK |
| | | | | EVEQDPGPLSVPEGAIVSLNCTYSNS |
| | | | | AFQYFMWYRQYSRKGPELLMYTYS |
| | | | | SGNKEDGRFTAQVDKSSKYISLFIRD |
| | | | | SQPSDSATYLCAMMGDTGTASKLTF |
| | | | | GTGTRLQVTLDIQNPDPAVYQLRDS |
| | | | | KSSDKSVCLFTDFDSQTNVSQSKDS |
| | | | | DVYITDKTVLDMRSMDFKSNSAVA |
| | | | | WSNKSDFACANAFNNSIIPEDTFFPS |
| | | | | PESSCDVKLVEKSFETDTNLNFQNLS |
| | | | | VIGFRILLLKVAGFNLLMTLRLWSS |
| 103 | R55P1G7 | beta | CDR1 | MDHEN |
| 104 | R55P1G7 | beta | CDR2 | SYDVKM |
| 105 | R55P1G7 | beta | CDR3 | CASSFGGYEQYF |
| 106 | R55P1G7 | beta | variable domain | MGIRLLCRVAFCFLAVGLVDVKVTQ |
| | | | | SSRYLVKRTGEKVFLECVQDMDHE |
| | | | | NMFWYRQDPGLGLRLIYFSYDVKM |
| | | | | KEKGDIPEGYSVSREKKERFSLILES |
| | | | | ASTNQTSMYLCASSFGGYEQYFGPG |
| | | | | TRLTVT |
| 107 | R55P1G7 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 108 | R55P157 | beta | full-length | MGIRLLCRVAFCFLAVGLVDVKVTQ |
| | | | | SSRYLVKRTGEKVFLECVQDMDHE |
| | | | | NMFWYRQDPGLGLRLIYFSYDVKM |
| | | | | KEKGDIPEGYSVSREKKERFSLILES |
| | | | | ASTNQTSMYLCASSFGGYEQYFGPG |
| | | | | TRLTVTEDLKNVFPPEVAVFEPSEAE |
| | | | | ISHTQKATLVCLATGFYPDHVELSW |
| | | | | WVNGKEVHSGVSTDPQPLKEQPAL |
| | | | | NDSRYCLSSRLRVSATFWQNPRNHF |
| | | | | RCQVQFYGLSENDEWTQDRAKPVT |
| | | | | QIVSAEAWGRADCGFTSESYQQGVL |
| | | | | SATILYEILLGKATLYAVLVSALVLM |
| | | | | AMVKRKDSRG |
| 109 | R59P2A7 | alpha | CDR1 | DRGSQS |
| 110 | R59P2A7 | alpha | CDR2 | IY |
| 111 | R59P2A7 | alpha | CDR3 | CAVQPHDMRF |
| 112 | R59P2A7 | alpha | variable domain | MKSLRVLLVILWLQLSWVWSQQKE |
| | | | | VEQNSGPLSVPEGAIASLNCTYSDRG |
| | | | | SQSFFWYRQYSGKSPELIMSIYSNGD |
| | | | | KEDGRFTAQLNKASQYVSLLIRDSQ |
| | | | | PSDSATYLCAVQPHDMRFGAGTRLT |
| | | | | VKP |
| 113 | R59P2A7 | alpha | constant domain | NIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 114 | R59P2A7 | alpha | full-length | MKSLRVLLVILWLQLSWVWSQQKE |
| | | | | VEQNSGPLSVPEGAIASLNCTYSDRG |
| | | | | SQSFFWYRQYSGKSPELIMSIYSNGD |
| | | | | KEDGRFTAQLNKASQYVSLLIRDSQ |
| | | | | PSDSATYLCAVQPHDMRFGAGTRLT |
| | | | | VKPNIQNPDPAVYQLRDSKSSDKSV |
| | | | | CLFTDFDSQTNVSQSKDSDVYITDKT |
| | | | | VLDMRSMDFKSNSAVAWSNKSDFA |
| | | | | CANAFNNSIIPEDTFFPSPESSCDVKL |
| | | | | VEKSFETDTNLNFQNLSVIGFRILLL |
| | | | | KVAGFNLLMTLRLWSS |
| 115 | R59P2A7 | beta | CDR1 | GTSNPN |
| 116 | R59P2A7 | beta | CDR2 | SVGIG |
| 117 | R59P2A7 | beta | CDR3 | CAWSGLVAEQFF |
| 118 | R59P2A7 | beta | variable domain | MLCSLLALLLGTFFGVRSQTIHQWP |
| | | | | ATLVQPVGSPLSLECTVEGTSNPNLY |
| | | | | WYRQAAGRGLQLLFYSVGIGQISSE |
| | | | | VPQNLSASRPQDRQFILSSKKLLLSD |
| | | | | SGFYLCAWSGLVAEQFFGPGTRLTV |
| | | | | L |
| 119 | R59P2A7 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 120 | R59P2A7 | beta | full-length | MLCSLLALLLGTFFGVRSQTIHQWP |
| | | | | ATLVQPVGSPLSLECTVEGTSNPNLY |
| | | | | WYRQAAGRGLQLLFYSVGIGQISSE |
| | | | | VPQNLSASRPQDRQFILSSKKLLLSD |
| | | | | SGFYLCAWSGLVAEQFFGPGTRLTV |
| | | | | LEDLKNVFPPEVAVFEPSEAEISHTQ |
| | | | | KATLVCLATGFYPDHVELSWWVNG |
| | | | | KEVHSGVSTDPQPLKEQPALNDSRY |
| | | | | CLSSRLRVSATFWQNPRNHFRCQVQ |
| | | | | FYGLSENDEWTQDRAKPVTQIVSAE |
| | | | | AWGRADCGFTSESYQQGVLSATILY |
| | | | | EILLGKATLYAVLVSALVLMAMVK |
| | | | | RKDSRG |
| 121 | 1G4 | alpha | CDR1 | DSAIYN |
| 122 | 1G4 | alpha | CDR2 | IQS |
| 123 | 1G4 | alpha | CDR3 | CAVRPTSGGSYIPTF |
| 124 | 1G4 | alpha | variable domain | METLLGLLILWLQLQWVSSKQEVTQ |
| | | | | IPAALSVPEGENLVLNCSFTDSAIYN |
| | | | | LQWFRQDPGKGLTSLLLIQSSQREQT |
| | | | | SGRLNASLDKSSGRSTLYIAASQPGD |
| | | | | SATYLCAVRPTSGGSYIPTFGRGTSLI |
| | | | | VHP |
| 125 | 1G4 | alpha | constant domain | YIQNPDPAVYQLRDSKSSDKSVCLFT |
| | | | | DFDSQTNVSQSKDSDVYITDKTVLD |
| | | | | MRSMDFKSNSAVAWSNKSDFACAN |
| | | | | AFNNSIIPEDTFFPSPESSCDVKLVEK |
| | | | | SFETDTNLNFQNLSVIGFRILLLKVA |
| | | | | GFNLLMTLRLWSS |
| 126 | 1G4 | alpha | full-length | METLLGLLILWLQLQWVSSKQEVTQ |
| | | | | IPAALSVPEGENLVLNCSFTDSAIYN |
| | | | | LQWFRQDPGKGLTSLLLIQSSQREQT |
| | | | | SGRLNASLDKSSGRSTLYIAASQPGD |
| | | | | SATYLCAVRPTSGGSYIPTFGRGTSLI |
| | | | | VHPYIQNPDPAVYQLRDSKSSDKSV |
| | | | | CLFTDFDSQTNVSQSKDSDVYITDKT |
| | | | | VLDMRSMDFKSNSAVAWSNKSDFA |
| | | | | CANAFNNSIIPEDTFFPSPESSCDVKL |
| | | | | VEKSFETDTNLNFQNLSVIGFRILLL |
| | | | | KVAGFNLLMTLRLWSS |
| 127 | 1G4 | beta | CDR1 | MNHEY |
| 128 | 1G4 | beta | CDR2 | SVGAGI |
| 129 | 1G4 | beta | CDR3 | CASSYVGNTGELFF |
| 130 | 1G4 | beta | variable domain | MSIGLLCCAALSLLWAGPVNAGVTQ |
| | | | | TPKFQVLKTGQSMTLQCAQDMNHE |
| | | | | YMSWYRQDPGMGLRLIHYSVGAGI |
| | | | | TDQGEVPNGYNVSRSTTEDFPLRLLS |
| | | | | AAPSQTSVYFCASSYVGNTGELFFG |
| | | | | EGSRLTVL |
| 131 | 1G4 | beta | constant domain | EDLKNVFPPEVAVFEPSEAEISHTQK |
| | | | | ATLVCLATGFYPDHVELSWWVNGK |
| | | | | EVHSGVSTDPQPLKEQPALNDSRYC |
| | | | | LSSRLRVSATFWQNPRNHFRCQVQF |
| | | | | YGLSENDEWTQDRAKPVTQIVSAEA |
| | | | | WGRADCGFTSESYQQGVLSATILYEI |
| | | | | LLGKATLYAVLVSALVLMAMVKRK |
| | | | | DSRG |
| 132 | 1G4 | beta | full-length | MSIGLLCCAALSLLWAGPVNAGVTQ |
| | | | | TPKFQVLKTGQSMTLQCAQDMNHE |
| | | | | YMSWYRQDPGMGLRLIHYSVGAGI |
| | | | | TDQGEVPNGYNVSRSTTEDFPLRLLS |
| | | | | AAPSQTSVYFCASSYVGNTGELFFG |
| | | | | EGSRLTVLEDLKNVFPPEVAVFEPSE |
| | | | | AEISHTQKATLVCLATGFYPDHVELS |
| | | | | WWVNGKEVHSGVSTDPQPLKEQPA |
| | | | | LNDSRYCLSSRLRVSATFWQNPRNH |
| | | | | FRCQVQFYGLSENDEWTQDRAKPV |
| | | | | TQIVSAEAWGRADCGFTSESYQQGV |
| | | | | LSATILYEILLGKATLYAVLVSALVL |
| | | | | MAMVKRKDSRG |

TABLE 2
Peptide sequences of the invention

| Peptide Code | Sequence | SEQ ID NO: |
|---|---|---|
| SPINK2-001 | ALSVLRLAL | 133 |
| SPINK2-001_A2 | AASVLRLAL | 134 |
| SPINK2-001_A3 | ALAVLRLAL | 135 |
| SPINK2-001_A4 | ALSALRLAL | 136 |
| SPINK2-001_A5 | ALSVARLAL | 137 |
| SPINK2-001_A6 | ALSVLALAL | 138 |
| SPINK2-001_A7 | ALSVLRAAL | 139 |
| SPINK2-001_A9 | ALSVLRLAA | 140 |
| SPINK2-001_T1 | TLSVLRLAL | 141 |
| SPINK2-001_T2 | ATSVLRLAL | 142 |
| SPINK2-001_T3 | ALTVLRLAL | 143 |
| SPINK2-001_T4 | ALSTLRLAL | 144 |
| SPINK2-001_T5 | ALSVTRLAL | 145 |
| SPINK2-001_T6 | ALSVLTLAL | 146 |
| SPINK2-001_T7 | ALSVLRTAL | 147 |
| SPINK2-001_T8 | ALSVLRLTL | 148 |
| SPINK2-001_T9 | ALSVLRLAT | 149 |
| CYP-003 | ALMNMKLAL | 150 |
| BAG6-001 | ALSDLRCNL | 151 |
| XRCC5-001 | ALSSLIHAL | 152 |
| TMEM147-001 | ALSTLALYV | 153 |
| SEC-001 | ALSVLADFL | 154 |
| GMPPA-001 | ALYASRLYL | 155 |
| EXOC4-002 | GLSDLRLEL | 156 |
| ARFGAP3-001 | IVSSLRLAY | 157 |
| ANKRD29-002 | YLDVIRLLL | 158 |
| NYESO1-001 | SLLMWITQV | 159 |

### EXAMPLES

Ten SPINK2-001-specific TCRs (R39P1C12, R39P1F5, R40P1C2, R41P3E6,R43P3G4, R44P3B3, R44P3E7, R49P2B7, R55P1G7 and R59P2A7, see Table 1), each encoding tumor specific TCR-alpha and TCR-beta chains, were isolated and amplified from T-cells of healthy donors. Cells from healthy donors were in vitro stimulated according to a method previously described (Walter et al., 2003 J Immunol., November 15;171(10):4974-8) and target-specific cells were single-cell sorted using HLA-A*02 multimers and then used for subsequent TCR isolation. TCR sequences were isolated via 5′ RACE by standard methods as described by e.g. Molecular Cloning a laboratory manual fourth edition by Green and Sambrook. The alpha and beta variable regions of TCRs R39P1C12, R39P1F5, R40P1C2, R41P3E6, R43P3G4, R44P3B3, R44P3E7, R49P2B7, R55P1G7 and R59P2A7 were sequenced and expression constructs were generated by gene synthesis for further functional characterization.

R41P3E6, R43P3G4, R55P1G7 and R59P2A7 are derived from HLA-A*02 negative donor (allo-reactive setting) and R39P1C12, R39P1F5, R40P1C2, R44P3B3, R44P3E7 and R49P2B7 are derived from a HLA-A*02 positive donor.

TCRs of interest were expressed in human T cells, e.g. through mRNA electroporation. The T cells were assessed for IFN-γ release after co-culture with different target cells, such as T2 cells loaded with different peptides as well as tumor cell lines. For T-cell activation, data is either shown as absolute IFNγ levels or as background subtracted data, indicated below. Efficacy of CD8+ T cells expressing TCRs R55P1G7 and R44P3B3 was determined e.g. by T cell activation studies (IFNγ release) using different tumor cell lines as target cells.

Background subtraction method for IFNγ release:

Meanbg(TCRoi; co)=[mean(TCRoi; co)−mean(TCRoi; effector only)]−[mean(mock; co)−mean(mock; effector only)]

The respective SDbg was calculated:

SDbg(TCRoi; co)=[SD(TCRoi; co)²+SD(TCRoi; effector only)²+SD(mock;co)²+SD(mock; effector only)²]∧[1/2]
TCRoi=effector cells expressing TCR of interest
Mock=effector cells without exogenous TCR expression
Co=effector cells co-cultured with target cells
Effector only=effector cells not co-cultured
Mean(bg)=mean IFNy release (background subtracted)
SD(bg)=standard deviation (background subtracted)
BLI (Biolayer interferometry) binding analysis for SPINK2-001 TCRs, expressed as soluble TCRs according to a previously described method (Willcox BE et al., 1999 Protein Sci., November;8(11):2418-23), and HLA-A*02/SPINK2-001 complex was used for affinity determination. BLI binding data for 1G4 TCR and HLA-A*02/NYESO1-001 are used as control.

### Example 1: T-Cell Receptor R39P1C12

TCR R39P1C12 (SEQ ID NOs: 1-12) is restricted towards HLA-A*02-presented SPINK2-001 (SEQ ID NO: 133) (see FIG. 11).

R39P1C12 specifically recognizes SPINK2-001 as human primary CD8+ T-cells re-expressing this TCR release IFNγ upon co-incubation with HLA-A*02+ target cells, loaded either with SPINK2-001 peptide or alanine or threonine substitution variants of SPINK2-001 (FIG. 1) or different peptides showing high degree of sequence similarity to SPINK2-001 (FIG. 11). NYESO1-001 peptide is used as negative control. TCR R39P1C12 has an EC₅₀ of 0.81 nM (FIG. 21) and an affinity of 18 μM (R²=0.9956) (FIG. 33A).

### Example 2: T-Cell Receptor R39P1F5

TCR R39P1F5 (SEQ ID NOs: 13-24) is restricted towards HLA-A*02-presented SPINK2-001 (SEQ ID NO: 133) (see FIG. 12).

R39P1F5 specifically recognizes SPINK2-001 as human primary CD8+ T-cells re-expressing this TCR release IFNγ upon co-incubation with HLA-A*02+ target cells, loaded either with SPINK2-001 peptide or alanine or threonine substitution variants of SPINK2-001 (FIG. 2) or different peptides showing high degree of sequence similarity to SPINK2-001 (FIG. 12). NYESO1-001 peptide is used as negative control. TCR R39P1F5 has an EC₅₀ of 1.52 nM (FIG. 22) and an affinity of 34 μM (R²=0.9962) (FIG. 33A).

### Example 3: T-Cell Receptor R40P1C2

TCR R40P1C2 (SEQ ID NOs: 25-36) is restricted towards HLA-A*02-presented SPINK2-001 (SEQ ID NO: 133) (see FIG. 13).

R40P1C2 specifically recognizes SPINK2-001 as human primary CD8+ T-cells re-expressing this TCR release IFNγ upon co-incubation with HLA-A*02+ target cells, loaded either with SPINK2-001 peptide or alanine or threonine substitution variants of SPINK2-001 (FIG. 3) or different peptides showing high degree of sequence similarity to SPINK2-001 (FIG. 13). NYESO1-001 peptide is used as negative control. TCR R40P1C2 has an EC₅₀ of 1.94 nM (FIG. 23).

### Example 4: T-Cell Receptor R41P3E6

TCR R41P3E6 (SEQ ID NOs: 37-48) is restricted towards HLA-A*02-presented SPINK2-001 (SEQ ID NO: 133) (see FIG. 14).

R41P3E6 specifically recognizes SPINK2-001 as human primary CD8+ T-cells re-expressing this TCR release IFNγ upon co-incubation with HLA-A*02+ target cells and bind HLA-A*02 tetramers (FIG. 31), respectively, loaded either with SPINK2-001 peptide or alanine or threonine substitution variants of SPINK2-001 (FIG. 4) or different peptides showing high degree of sequence similarity to SPINK2-001 (FIG. 14). NYESO1-001 peptide is used as negative control. TCR R41P3E6 has an EC₅₀ of 1.03 nM (FIG. 24) and an affinity of 13 μM (R²=0.9892) (FIG. 33A).

### Example 5: T-Cell Receptor R43P3G4

TCR R43P3G4 (SEQ ID NOs: 49-60) is restricted towards HLA-A*02-presented SPINK2-001 (SEQ ID NO: 133) (see FIG. 15).

R43P3G4 specifically recognizes SPINK2-001 as human primary CD8+ T-cells re-expressing this TCR release IFNγ upon co-incubation with HLA-A*02+ target cells, loaded either with SPINK2-001 peptide or alanine or threonine substitution variants of SPINK2-001 (FIG. 5) or different peptides showing high degree of sequence similarity to SPINK2-001 (FIG. 15). NYESO1-001 peptide is used as negative control. TCR R43P3G4 has an EC₅₀ of 1.34 nM (FIG. 25).

### Example 6: T-Cell Receptor R44P3B3

TCR R44P3B3 (SEQ ID NOs: 61-72) is restricted towards HLA-A*02-presented SPINK2-001 (SEQ ID NO: 133) (see FIG. 16).

R44P3B3 specifically recognizes SPINK2-001 as human primary CD8+ T-cells re-expressing this TCR release IFNγ upon co-incubation with HLA-A*02+ target cells and bind HLA-A*02 tetramers (FIG. 31), respectively, loaded either with SPINK2-001 peptide or alanine or threonine substitution variants of SPINK2-001 (FIG. 6) or different peptides showing high degree of sequence similarity to SPINK2-001 (FIG. 16). NYESO1-001 peptide is used as negative control. TCR R44P3B3 has an EC₅₀ of 1.06 nM (FIG. 26) and an affinity of 37 μM (R²=0.9947) (FIG. 33B).

For CD8+ T cells expressing TCR R44P3B3, an activity towards the SPINK2-001-overexpressing A375 tumor cells was observed (FIG. 32), but not towards the wildtype A375 tumor cell line. A375 cells endogenously express HLA-A2.

T-cell activation upon co-culture with cell lines expressing HLA-A*02 and SPINK2-001 reflects the recognition of endogenously presented target pHLA (peptide presented on human leukocyte antigen) by TCR R44P3B3.

### Example 7: T-Cell Receptor R44P3E7

TCR R44P3E7 (SEQ ID NOS: 73-84) is restricted towards HLA-A*02-presented SPINK2-001 (SEQ ID NO: 133) (see FIG. 17).

R44P3E7 specifically recognizes SPINK2-001 as human primary CD8+ T-cells re-expressing this TCR release IFNγ upon co-incubation with HLA-A*02+ target cells, loaded either with SPINK2-001 peptide or alanine or threonine substitution variants of SPINK2-001 (FIG. 7) or different peptides showing high degree of sequence similarity to SPINK2-001 (FIG. 17). NYESO1-001 peptide is used as negative control. TCR R44P3E7 has an EC₅₀ of 0.86 nM (FIG. 27).

### Example 8: T-Cell Receptor R49P2A7

TCR R49P2A7 (SEQ ID NOs: 85-96) is restricted towards HLA-A*02-presented SPINK2-001 (SEQ ID NO: 133) (see FIG. 18).

R49P2A7 specifically recognizes SPINK2-001 as human primary CD8+ T-cells re-expressing this TCR release IFNγ upon co-incubation with HLA-A*02+ target cells and bind HLA-A*02 tetramers (FIG. 31), respectively, loaded either with SPINK2-001 peptide or alanine or threonine substitution variants of SPINK2-001 (FIG. 8) or different peptides showing high degree of sequence similarity to SPINK2-001 (FIG. 18). NYESO1-001 peptide is used as negative control. TCR R49P2A7 has an EC₅₀ of >83.24 nM (FIG. 28).

### Example 9: T-Cell Receptor R55P1G7

TCR R55P1G7 (SEQ ID NOs: 97-108) is restricted towards HLA-A*02-presented SPINK2-001 (SEQ ID NO: 133) (see FIG. 19).

R55P1G7 specifically recognizes SPINK2-001 as human primary CD8+ T-cells re-expressing this TCR release IFNγ upon co-incubation with HLA-A*02+ target cells and bind HLA-A*02 tetramers (FIG. 31), respectively, loaded either with SPINK2-001 peptide or alanine or threonine substitution variants of SPINK2-001 (FIG. 9) or different peptides showing high degree of sequence similarity to SPINK2-001 (FIG. 19). NYESO1-001 peptide is used as negative control. TCR R55P1G7 has an EC₅₀ of 91.5 pM (FIG. 29) and an affinity of 4.3 μM (R²=0.9765) (FIG. 33B).

For CD8+ T cells expressing TCR R55P1G7, an activity towards the SPINK2-001-overexpressing A375 tumor cells was observed (FIG. 32), but not towards the wildtype A375 tumor cell line. The A375 cells endogenously express HLA-A2.

T-cell activation upon co-culture with cell lines expressing HLA-A*02 and SPINK2-001 reflects the recognition of endogenously presented target pHLA (peptide presented on human leukocyte antigen) by TCR R55P1G7.

### Example 10: T-Cell Receptor R59P2A7

TCR R59P2A7 (SEQ ID NOs: 109-120) is restricted towards HLA-A*02-presented SPINK2-001 (SEQ ID NO: 133) (see FIG. 20).

R59P2A7 specifically recognizes SPINK2-001 as human primary CD8+ T-cells re-expressing this TCR release IFNγ upon co-incubation with HLA-A*02+ target cells, loaded either with SPINK2-001 peptide or alanine or threonine substitution variants of SPINK2-001 (FIG. 10) or different peptides showing high degree of sequence similarity to SPINK2-001 (FIG. 20). NYESO1-001 peptide is used as negative control. TCR R59P2A7 has an EC₅₀ of 0.86 nM (FIG. 30).

1. An antigen recognizing construct comprising at least one complementary determining region (CDR) 3 having at least 80% sequence identity to an amino acid sequence selected from SEQ ID NOs: 3, 9, 15, 21, 27, 33, 39, 45, 51, 57, 63, 69, 75, 81, 87, 93, 99, 105, 111, and 117.

2. The antigen recognizing construct according to claim 1, wherein said antigen recognizing construct is capable of specifically and/or selectively binding to a SPINK2-001 antigenic peptide.

3. The antigen recognizing construct according to claim 1, wherein the antigen recognizing construct is an antibody, or derivative or fragment thereof, or a T cell receptor (TCR), or a derivative or fragment thereof.

4. The antigen recognizing construct according to claim 1, comprising a TCR α or γ chain; and/or a TCR β or δ chain; wherein the TCR α or γ chain comprises a CDR3 having at least 80% sequence identity to an amino acid sequence selected from SEQ ID NOs: 3, 15, 27, 39, 51, 63, 75, 87, 99, and 111, and/or wherein the TCR β or δ chain comprises a CDR3 having at least 80% sequence identity to an amino acid sequence selected from SEQ ID NOs: 9, 21, 33, 45, 57, 69, 81, 93, 105, and 117.

5. The antigen recognizing construct according to claim 4, wherein the TCR α or γ chain further comprises a CDR1 having at least 80% sequence identity to an amino acid sequence selected from SEQ ID NOs: 1, 13, 25, 37, 49, 61, 73, 85, 97, and 109; and/or a CDR2 having at least 80% sequence identity to an amino acid sequence selected from SEQ ID NOs: 2, 14, 26, 38, 50, 62, 74, 86, 98, and 110.

6. The antigen recognizing construct according to claim 4, wherein the TCR β or δ chain further comprises a CDR1 having at least 80% sequence identity to an amino acid sequence selected from SEQ ID NOs: 7, 19, 31, 43, 55, 67, 79, 91, 103, and 115; and/or a CDR2 having at least 80% sequence identity to an amino acid sequence selected from SEQ ID NOs: 8, 20, 32, 44, 56, 68, 80, 92, 104, and 116.

7. The antigen recognizing construct according to claim 1, comprising a TCR variable chain region having at least 80% sequence identity to an amino acid sequence selected from SEQ ID NOs: 4, 10, 16, 22, 28, 34, 40, 46, 52, 58, 64, 70, 76, 82, 88, 94, 100, 106, 112, and 118.

8. The antigen recognizing construct according to claim 1, comprising a binding fragment of a TCR, and wherein said binding fragment comprises CDR1 to CDR3 optionally selected from the CDR1 to CDR3 sequences having the amino acid sequences of SEQ ID NOs: 1, 2, 3; or 7, 8, 9; or 13, 14, 15; or 19, 20, 21; or 25, 26, 27; or 31, 32, 33; or 37, 38, 39; or 43, 44, 45; or 49, 50, 51; or 55, 56, 57; or 61, 62, 63; or 67, 68, 69; or 73, 74, 75; or 79, 80, 81; or 85, 86, 87; or 91, 92, 93; or 97, 98, 99; or 103, 104, 105; or 109, 110, 111; or 115, 116, 117.

9. The antigen recognizing construct according to claim 1, wherein in the variable domain of the a or β chain, an amino acid at position 44 according to the IMGT numbering is substituted with another suitable amino acid thereby improving stability and/or pairing of said chains.

10. A nucleic acid encoding for an antigen recognizing construct according to claim 1.

11. A vector comprising a nucleic acid according to claim 10.

12. A host cell comprising an antigen recognizing construct according to claim 1, or a nucleic acid encoding said antigen recognizing construct, or a vector comprising said nucleic acid, optionally the host cell is a lymphocyte, optionally a T lymphocyte or T lymphocyte progenitor, optionally a CD4 or CD8 positive T-cell.

13. A pharmaceutical composition comprising the antigen recognizing construct according to claim 1, or a nucleic acid encoding said antigen recognizing construct, or a vector comprising said nucleic acid, or the host cell comprising said antigen recognizing construct, and a pharmaceutical acceptable carrier, stabilizer and/or excipient.

14. The antigen recognizing construct according to claim 1, or a nucleic acid encoding said antigen recognizing construct, or vector, or a host cell comprising said antigen recognizing construct, or the pharmaceutical composition thereof, for use in medicine, optionally for use in the diagnosis, prevention, and/or treatment of a proliferative disease.

15. A method of manufacturing a TAA specific antigen recognizing construct expressing cell line, comprising
a. providing a suitable host cell,
b. providing a genetic construct comprising a coding sequence encoding the antigen recognizing construct according to claim 1,
c. introducing into said suitable host cell said genetic construct,
d. expressing said genetic construct by said suitable host cell.

16. The method according to claim 15, further comprising isolation and purification of the antigen recognizing construct from the suitable host cell and, optionally, reconstitution of the antigen recognizing construct in a T-cell.

17. The antigen recognizing construct of claim 1, comprising at least one CDR 3 having at least 90% sequence identity to an amino acid sequence selected from SEQ ID NOs: 3, 9, 15, 21, 27, 33, 39, 45, 51, 57, 63, 69, 75, 81, 87, 93, 99, 105, 111, and 117.

18. The antigen recognizing construct of claim 1, comprising at least one CDR 3 having an amino acid sequence selected from SEQ ID NOs: 3, 9, 15, 21, 27, 33, 39, 45, 51, 57, 63, 69, 75, 81, 87, 93, 99, 105, 111, and 117.

19. The antigen recognizing construct according to claim 7, comprising a TCR variable chain region having at least 90% sequence identity to an amino acid sequence selected from SEQ ID NOs: 4, 10, 16, 22, 28, 34, 40, 46, 52, 58, 64, 70, 76, 82, 88, 94, 100, 106, 112, and 118.

20. The antigen recognizing construct according to claim 7, comprising a TCR variable chain region having an amino acid sequence selected from SEQ ID NOs: 4, 10, 16, 22, 28, 34, 40, 46, 52, 58, 64, 70, 76, 82, 88, 94, 100, 106, 112, and 118.


SEQUENCE LISTING (159 sequences)

SEQ ID NO: 1 - protein, 6 aa, Homo sapiens
DSSSTY

SEQ ID NO: 2 - skipped

SEQ ID NO: 3 - protein, 13 aa, Homo sapiens
CAEIDNQGGKLIF

SEQ ID NO: 4 - protein, 132 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEIDNQGGKLIFGQGTELSVKP

SEQ ID NO: 5 - protein, 141 aa, Homo sapiens
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - protein, 273 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEIDNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 7 - protein, 5 aa, Homo sapiens
SGHDT

SEQ ID NO: 8 - protein, 6 aa, Homo sapiens
YYEEEE

SEQ ID NO: 9 - protein, 12 aa, Homo sapiens
CASSQLNTEAFF

SEQ ID NO: 10 - protein, 130 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSQLNTEAFFGQGTRLTVV

SEQ ID NO: 11 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 12 - protein, 307 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSQLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 13 - protein, 6 aa, Homo sapiens
DRGSQS

SEQ ID NO: 14 - skipped

SEQ ID NO: 15 - protein, 10 aa, Homo sapiens
CAVNNARLMF

SEQ ID NO: 16 - protein, 129 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNNARLMFGDGTQLVVKP

SEQ ID NO: 17 - protein, 141 aa, Homo sapiens; identical to SEQ ID NO: 5

SEQ ID NO: 18 - protein, 270 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - protein, 5 aa, Homo sapiens
SNHLY

SEQ ID NO: 20 - protein, 6 aa, Homo sapiens
FYNNEI

SEQ ID NO: 21 - protein, 13 aa, Homo sapiens
CASSGQGANEQYF

SEQ ID NO: 22 - protein, 132 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGANEQYFGPGTRLTVT

SEQ ID NO: 23 - protein, 179 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 24 - protein, 311 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGANEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 25 - protein, 7 aa, Homo sapiens
TSESDYY

SEQ ID NO: 26 - protein, 4 aa, Homo sapiens
QEAY

SEQ ID NO: 27 - protein, 10 aa, Homo sapiens
CAYLNYQLIW

SEQ ID NO: 28 - protein, 131 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLNYQLIWGAGTKLIIKP

SEQ ID NO: 29 - protein, 141 aa, Homo sapiens
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 272 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 31 - protein, 5 aa, Homo sapiens
SNHLY

SEQ ID NO: 32 - protein, 6 aa, Homo sapiens
FYNNEI

SEQ ID NO: 33 - protein, 13 aa, Homo sapiens
CASSEMTAVGQYF

SEQ ID NO: 34 - protein, 132 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEMTAVGQYFGPGTRLTVT

SEQ ID NO: 35 - protein, 179 aa, Homo sapiens; identical to SEQ ID NO: 23

SEQ ID NO: 36 - protein, 311 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEMTAVGQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 37 - protein, 6 aa, Homo sapiens
DRGSQS

SEQ ID NO: 38 - skipped

SEQ ID NO: 39 - protein, 11 aa, Homo sapiens
CAAFSGYALNF

SEQ ID NO: 40 - protein, 130 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAFSGYALNFGKGTSLLVTP

SEQ ID NO: 41 - protein, 141 aa, Homo sapiens
HIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 271 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAFSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 43 - protein, 5 aa, Homo sapiens
SNHLY

SEQ ID NO: 44 - protein, 6 aa, Homo sapiens
FYNNEI

SEQ ID NO: 45 - protein, 12 aa, Homo sapiens
CASSQYTGELFF

SEQ ID NO: 46 - protein, 131 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSQYTGELFFGEGSRLTVL

SEQ ID NO: 47 - protein, 179 aa, Homo sapiens; identical to SEQ ID NO: 23

SEQ ID NO: 48 - protein, 310 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSQYTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 49 - protein, 6 aa, Homo sapiens
DRGSQS

SEQ ID NO: 50 - skipped

SEQ ID NO: 51 - protein, 10 aa, Homo sapiens
CAVNGGDMRF

SEQ ID NO: 52 - protein, 129 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNGGDMRFGAGTRLTVKP

SEQ ID NO: 53 - protein, 141 aa, Homo sapiens; identical to SEQ ID NO: 5

SEQ ID NO: 54 - protein, 270 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNGGDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 55 - protein, 5 aa, Homo sapiens
SNHLY

SEQ ID NO: 56 - protein, 6 aa, Homo sapiens
FYNNEI

SEQ ID NO: 57 - protein, 13 aa, Homo sapiens
CASSGQGALEQYF

SEQ ID NO: 58 - protein, 132 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGALEQYFGPGTRLTVT

SEQ ID NO: 59 - protein, 179 aa, Homo sapiens; identical to SEQ ID NO: 23

SEQ ID NO: 60 - protein, 311 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGALEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - protein, 6 aa, Homo sapiens
NSMFDY

SEQ ID NO: 62 - skipped

SEQ ID NO: 63 - protein, 15 aa, Homo sapiens
CAASGLYNQGGKLIF

SEQ ID NO: 64 - protein, 140 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGLYNQGGKLIFGQGTELSVKP

SEQ ID NO: 65 - protein, 141 aa, Homo sapiens; identical to SEQ ID NO: 5

SEQ ID NO: 66 - protein, 281 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGLYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 67 - protein, 5 aa, Homo sapiens
LGHDT

SEQ ID NO: 68 - protein, 6 aa, Homo sapiens
YNNKEL

SEQ ID NO: 69 - protein, 14 aa, Homo sapiens
CASSLGDRGYEQYF

SEQ ID NO: 70 - protein, 132 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSLGDRGYEQYFGPGTRLTVT

SEQ ID NO: 71 - protein, 179 aa, Homo sapiens; identical to SEQ ID NO: 23

SEQ ID NO: 72 - protein, 311 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSLGDRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - protein, 6 aa, Homo sapiens
DSSSTY

SEQ ID NO: 74 - skipped

SEQ ID NO: 75 - protein, 12 aa, Homo sapiens
CAEINNNARLMF

SEQ ID NO: 76 - protein, 131 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEINNNARLMFGDGTQLVVKP

SEQ ID NO: 77 - protein, 141 aa, Homo sapiens; identical to SEQ ID NO: 5

SEQ ID NO: 78 - protein, 272 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEINNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 79 - protein, 5 aa, Homo sapiens
PRHDT

SEQ ID NO: 80 - protein, 6 aa, Homo sapiens
FYEKMQ

SEQ ID NO: 81 - protein, 13 aa, Homo sapiens
CASSPPDQNTQYF

SEQ ID NO: 82 - protein, 141 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPPDQNTQYFGPGTRLTVL

SEQ ID NO: 83 - protein, 179 aa, Homo sapiens; identical to SEQ ID NO: 23

SEQ ID NO: 84 - protein, 320 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPPDQNTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 85 - protein, 6 aa, Homo sapiens
SSVPPY

SEQ ID NO: 86 - protein, 4 aa, Homo sapiens
YTTG

SEQ ID NO: 87 - protein, 13 aa, Homo sapiens
CAVRIFGNEKLTF

SEQ ID NO: 88 - protein, 132 aa, Homo sapiens
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVRIFGNEKLTFGTGTRLTIIP

SEQ ID NO: 89 - protein, 141 aa, Homo sapiens; identical to SEQ ID NO: 5

SEQ ID NO: 90 - protein, 273 aa, Homo sapiens
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVRIFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 91 - protein, 5 aa, Homo sapiens
MDHEN

SEQ ID NO: 92 - protein, 6 aa, Homo sapiens
SYDVKM

SEQ ID NO: 93 - protein, 15 aa, Homo sapiens
CASSLMGELTGELFF

SEQ ID NO: 94 - protein, 133 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLMGELTGELFFGEGSRLTVL

SEQ ID NO: 95 - protein, 179 aa, Homo sapiens; identical to SEQ ID NO: 23

SEQ ID NO: 96 - protein, 312 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLMGELTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 97 - protein, 6 aa, Homo sapiens
NSAFQY

SEQ ID NO: 98 - skipped

SEQ ID NO: 99 - protein, 15 aa, Homo sapiens
CAMMGDTGTASKLTF

SEQ ID NO: 100 - protein, 135 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMMGDTGTASKLTFGTGTRLQVTL

SEQ ID NO: 101 - protein, 141 aa, Homo sapiens; identical to SEQ ID NO: 29

SEQ ID NO: 102 - protein, 276 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMMGDTGTASKLTFGTGTRLQVTLDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 103 - protein, 5 aa, Homo sapiens
MDHEN

SEQ ID NO: 104 - protein, 6 aa, Homo sapiens
SYDVKM

SEQ ID NO: 105 - protein, 12 aa, Homo sapiens
CASSFGGYEQYF

SEQ ID NO: 106 - protein, 130 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFGGYEQYFGPGTRLTVT

SEQ ID NO: 107 - protein, 179 aa, Homo sapiens; identical to SEQ ID NO: 23

SEQ ID NO: 108 - protein, 309 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFGGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 109 - protein, 6 aa, Homo sapiens
DRGSQS

SEQ ID NO: 110 - skipped

SEQ ID NO: 111 - protein, 10 aa, Homo sapiens
CAVQPHDMRF

SEQ ID NO: 112 - protein, 129 aa, Homo sapiens
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVQPHDMRFGAGTRLTVKP

SEQ ID NO: 113 - protein, 141 aa, Homo sapiens; identical to SEQ ID NO: 5

SEQ ID NO: 114 - protein, 270 aa, Homo sapiens
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVQPHDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 115 - protein, 6 aa, Homo sapiens
GTSNPN

SEQ ID NO: 116 - protein, 5 aa, Homo sapiens
SVGIG

SEQ ID NO: 117 - protein, 12 aa, Homo sapiens
CAWSGLVAEQFF

SEQ ID NO: 118 - protein, 128 aa, Homo sapiens
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSGLVAEQFFGPGTRLTVL

SEQ ID NO: 119 - protein, 179 aa, Homo sapiens; identical to SEQ ID NO: 23

SEQ ID NO: 120 - protein, 307 aa, Homo sapiens
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSGLVAEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 121 - protein, 6 aa, Homo sapiens
DSAIYN

SEQ ID NO: 122 - skipped

SEQ ID NO: 123 - protein, 15 aa, Homo sapiens
CAVRPTSGGSYIPTF

SEQ ID NO: 124 - protein, 133 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHP

SEQ ID NO: 125 - protein, 141 aa, Homo sapiens
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 126 - protein, 274 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 127 - protein, 5 aa, Homo sapiens
MNHEY

SEQ ID NO: 128 - protein, 6 aa, Homo sapiens
SVGAGI

SEQ ID NO: 129 - protein, 14 aa, Homo sapiens
CASSYVGNTGELFF

SEQ ID NO: 130 - protein, 132 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 131 - protein, 179 aa, Homo sapiens; identical to SEQ ID NO: 23

SEQ ID NO: 132 - protein, 311 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 133 - protein, 9 aa, Homo sapiens
ALSVLRLAL

SEQ ID NO: 134 - protein, 9 aa, Homo sapiens
AASVLRLAL

SEQ ID NO: 135 - protein, 9 aa, Homo sapiens
ALAVLRLAL

SEQ ID NO: 136 - protein, 9 aa, Homo sapiens
ALSALRLAL

SEQ ID NO: 137 - protein, 9 aa, Homo sapiens
ALSVARLAL

SEQ ID NO: 138 - protein, 9 aa, Homo sapiens
ALSVLALAL

SEQ ID NO: 139 - protein, 9 aa, Homo sapiens
ALSVLRAAL

SEQ ID NO: 140 - protein, 9 aa, Homo sapiens
ALSVLRLAA

SEQ ID NO: 141 - protein, 9 aa, Homo sapiens
TLSVLRLAL

SEQ ID NO: 142 - protein, 9 aa, Homo sapiens
ATSVLRLAL

SEQ ID NO: 143 - protein, 9 aa, Homo sapiens
ALTVLRLAL

SEQ ID NO: 144 - protein, 9 aa, Homo sapiens
ALSTLRLAL

SEQ ID NO: 145 - protein, 9 aa, Homo sapiens
ALSVTRLAL

SEQ ID NO: 146 - protein, 9 aa, Homo sapiens
ALSVLTLAL

SEQ ID NO: 147 - protein, 9 aa, Homo sapiens
ALSVLRTAL

SEQ ID NO: 148 - protein, 9 aa, Homo sapiens
ALSVLRLTL

SEQ ID NO: 149 - protein, 9 aa, Homo sapiens
ALSVLRLAT

SEQ ID NO: 150 - protein, 9 aa, Homo sapiens
ALMNMKLAL

SEQ ID NO: 151 - protein, 9 aa, Homo sapiens
ALSDLRCNL

SEQ ID NO: 152 - protein, 9 aa, Homo sapiens
ALSSLIHAL

SEQ ID NO: 153 - protein, 9 aa, Homo sapiens
ALSTLALYV

SEQ ID NO: 154 - protein, 9 aa, Homo sapiens
ALSVLADFL

SEQ ID NO: 155 - protein, 9 aa, Homo sapiens
ALYASRLYL

SEQ ID NO: 156 - protein, 9 aa, Homo sapiens
GLSDLRLEL

SEQ ID NO: 157 - protein, 9 aa, Homo sapiens
IVSSLRLAY

SEQ ID NO: 158 - protein, 9 aa, Homo sapiens
YLDVIRLLL

SEQ ID NO: 159 - protein, 9 aa, Homo sapiens
SLLMWITQV